(19) United States
(12) Patent Application Publication
(10) Pub. No.: US 2023/0047753 A1
(43) Pub. Date: Feb. 16, 2023

(54) VIRAL VECTORS FOR ENHANCED ULTRASOUND-MEDIATED DELIVERY TO THE BRAIN

(71) Applicant: California Institute of Technology, Pasadena, CA (US)

(72) Inventors: Jerzy O. Szablowski, Pasadena, CA (US); Hongyi Li, Pasadena, CA (US); John E. Heath, Stanford, CA (US); Mikhail G. Shapiro, Pasadena, CA (US)

(21) Appl. No.: 17/814,384

(22) Filed: Jul. 22, 2022

Related U.S. Application Data
(60) Provisional application No. 63/225,006, filed on Jul. 23, 2021.

(51) Int. Cl. C12N 15/861

(52) U.S. Cl. CPC C12N 15/8616; C12N 2750/14143

(57) ABSTRACT

Disclosed herein include adeno-associated virus (AAV) acoustic targeting peptides. An AAV comprising the AAV acoustic targeting peptide can exhibit increased transduction at site(s) of focused ultrasound blood-brain barrier opening (FUS-BBBO), increased neuronal tropism, and diminished transduction of peripheral organs. Disclosed herein include recombinant adeno-associated virus (rAAV) comprising an AAV acoustic targeting peptide disclosed herein. Also provided herein include methods of delivering an agent to one or more target brain region(s) of a subject.

### RELATED APPLICATIONS

This application claims the benefit under 35 U.S.C. § 119(e) of U.S. Provisional Patent Application Ser. No. 63/225,006, filed Jul. 23, 2021, the content of this related application is incorporated herein by reference in its entirety for all purposes.

### STATEMENT REGARDING FEDERALLY SPONSORED R&D

This invention was made with government support under Grant No. MH120102 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ-302446-US, created Jul. 21, 2022, which is 28 kilobytes in size. The information in the electronic format of the Sequence Listing is incorporated herein by reference in its entirety.

### BACKGROUND

### Field

The present disclosure relates generally to the field of adeno-associated virus vectors. More specifically, the disclosure relates to methods and compositions for site-specific noninvasive gene delivery to the brain.

### Description of the Related Art

Targeted gene delivery to the brain is a critical tool for neuroscience research and has significant potential to treat human disease. However, the site-specific delivery of common gene vectors such as adeno-associated viruses (AAVs) is typically performed via invasive injections, limiting their scope of research and clinical applications. Alternatively, focused ultrasound blood-brain-barrier opening (FUS-BBBO), performed noninvasively, enables the site-specific entry of AAVs into the brain from systemic circulation. However, when used in conjunction with natural AAV serotypes, this approach has limited transduction efficiency, requires ultrasound parameters close to tissue damage limits, and results in undesirable transduction of peripheral organs.

Gene therapy is one of the most promising emerging approaches to treating human disease. Recently, a number of gene therapies were approved for clinical use, including blindness, muscular dystrophy, and metabolic disorders. Many of these therapies use adeno-associated viral vectors (AAVs) to deliver genes to various organs, but few target the brain. Although several neurological and psychiatric diseases could benefit from gene therapies targeting specific neural circuits, a key challenge limiting the development of such treatments is the need for invasive intracranial injections of the viral vectors. While recent advances are enabling brain-wide gene delivery from systemic or cerebrospinal fluid circulation, these approaches do not provide the spatial targeting needed to address regionally defined neural circuits.

Focused ultrasound blood-brain barrier opening (FUS-BBBO) is a recently developed technique with the potential to overcome these limitations by providing a route to noninvasive, site-specific gene delivery to the brain. In FUS-BBBO ultrasound is focused through an intact skull to transiently loosen tight junctions in the BBB and allow for the passage of AAVs from the blood into the targeted brain site. FUS-BBBO can target intravenously administered AAVs to millimeter-sized brain sites or cover large regions of the brain without tissue damage. These capabilities place FUS-BBBO in contrast with intracerebral injections, which are invasive and deliver genes to a single 2-3 millimeter-sized region per injection, requiring a large number of brain penetrations to cover larger regions of interest. At the same time, the spatial targeting capability of FUS-BBBO differentiates it from the use of spontaneously brain-penetrating engineered AAV serotypes, which lack spatial specificity. In proof of concept studies, FUS-BBBO has been used in rodents to introduce AAVs encoding reporter genes such as GFP, growth factors, optogenetic receptors. The delivery of chemogenetic receptors to the hippocampus provided the ability to modulate memory formation. Despite its promise, three critical drawbacks currently limit the potential of FUS-BBBO in research and therapy applications. First, while the BBB effectively prevents non-FUS-targeted regions of the brain from transduction by systemically administered AAV, peripheral organs have endothelia that allow AAV entry and consequently receive a high dose of the virus, which could lead to toxicity. Second, the relative inefficiency of AAV entry at the site of FUS-BBBO leads to the requirement of high doses of systemic AAVs, on the order of ^{˜}10¹⁰ viral particles per gram of body weight. While this magnitude has been used in recent clinical trials, it drives higher peripheral transduction and adds to the cost of potential therapies. Third, efficient delivery of AAV typically require acoustic parameters below, but close to, the threshold for brain tissue damage, reducing the margin for error in interventional planning. There is a need for compositions and methods wherein AAV exhibit increased transduction at site(s) of FUS-BBBO, increased neuronal tropism, and diminished transduction of peripheral organs.

### SUMMARY

Disclosed herein include adeno-associated virus (AAV) acoustic targeting peptides. In some embodiments, the adeno-associated virus (AAV) acoustic targeting peptide comprises an amino acid sequence that comprises at least 4 contiguous amino acids from a sequence selected from the group consisting of AGNTSDR (SEQ ID NO: 1; AAV.FUS.1), ATDAYNK (SEQ ID NO: 2; AAV.FUS.2), WSEGGQP (SEQ ID NO: 3; AAV.FUS.3), SVGSADP (SEQ ID NO: 4; AAV.FUS.4), and VRMEGEV (SEQ ID NO: 5; AAV.FUS.5).

In some embodiments, the AAV acoustic targeting peptide comprises at least 4 contiguous amino acids from the sequence of AGNTSDR (SEQ ID NO: 1). In some embodiments, the AAV acoustic targeting peptide comprises at least 5 contiguous amino acids from the sequence of AGNTSDR (SEQ ID NO: 1). In some embodiments, the AAV acoustic targeting peptide comprises at least 6 contiguous amino acids from the sequence of AGNTSDR (SEQ ID NO: 1). In some embodiments, the AAV acoustic targeting peptide comprises AGNTSDR (SEQ ID NO: 1).

In some embodiments, the AAV acoustic targeting peptide comprises at least 4 contiguous amino acids from the sequence ATDAYNK (SEQ ID NO: 2). In some embodiments, the AAV acoustic targeting peptide comprises at least 5 contiguous amino acids from the sequence of ATDAYNK (SEQ ID NO: 2). In some embodiments, the AAV acoustic targeting peptide comprises at least 6 contiguous amino acids from the sequence of ATDAYNK (SEQ ID NO: 2). In some embodiments, the AAV acoustic targeting peptide comprises ATDAYNK (SEQ ID NO: 2).

In some embodiments, the AAV acoustic targeting peptide comprises at least 4 contiguous amino acids from the sequence WSEGGQP (SEQ ID NO: 3). In some embodiments, the AAV acoustic targeting peptide comprises at least 5 contiguous amino acids from the sequence of WSEGGQP (SEQ ID NO: 3). In some embodiments, the AAV acoustic targeting peptide comprises at least 6 contiguous amino acids from the sequence of WSEGGQP (SEQ ID NO: 3). In some embodiments, the AAV acoustic targeting peptide comprises WSEGGQP (SEQ ID NO: 3).

In some embodiments, the AAV acoustic targeting peptide comprises at least 4 contiguous amino acids from the sequence SVGSADP (SEQ ID NO: 4). In some embodiments, the AAV acoustic targeting peptide comprises at least 5 contiguous amino acids from the sequence of SVGSADP (SEQ ID NO: 4). In some embodiments, the AAV acoustic targeting peptide comprises at least 6 contiguous amino acids from the sequence of SVGSADP (SEQ ID NO: 4). In some embodiments, the AAV acoustic targeting peptide comprises SVGSADP (SEQ ID NO: 4).

In some embodiments, the AAV acoustic targeting peptide comprises at least 4 contiguous amino acids from the sequence VRMEGEV (SEQ ID NO: 5). In some embodiments, the AAV acoustic targeting peptide comprises at least 5 contiguous amino acids from the sequence of VRMEGEV (SEQ ID NO: 5). In some embodiments, the AAV acoustic targeting peptide comprises at least 6 contiguous amino acids from the sequence of VRMEGEV (SEQ ID NO: 5). In some embodiments, the AAV acoustic targeting peptide comprises VRMEGEV (SEQ ID NO: 5).

In some embodiments, the AAV acoustic targeting peptide is part of an AAV. In some embodiments, the AAV acoustic targeting peptide is part of a capsid protein of the AAV. In some embodiments, the AAV acoustic targeting peptide is conjugated to a nanoparticle, a second molecule, a viral capsid protein, or a combination thereof. In some embodiments, the AAV acoustic targeting peptide is configured enhance the focused ultrasound (FUS)-target specificity of FUS blood-brain-barrier opening (FUS-BBBO)-mediated transduction.

In some embodiments, an AAV comprising the AAV acoustic targeting peptide demonstrates: an at least about 1.1-fold increase in transduction at site(s) of FUS-BBBO; an at least about 1.1-fold increase in neuronal tropism; and/or an at least about 1.1-fold decrease in transduction in peripheral organs, as compared to the corresponding parental AAV that does not comprise the acoustic targeting peptide.

Disclosed herein include AAV capsid proteins. In some embodiments, the AAV capsid protein comprises an AAV acoustic targeting peptide disclosed herein. In some embodiments, the AAV capsid is derived from AAV9, or a variant thereof. In some embodiments, the AAV capsid is derived from an AAV selected from the group consisting of AAV1, AAV2, AAV3, AAV3b, AAV4, AAV5, AAV6, AAV7, AAV8, AAV9, AAV10, human isolate hu.31, human isolate hu.32, rhesus isolate rh.8, and rhesus isolate rh.10. In some embodiments, the AAV capsid is derived from an AAV selected from the group consisting of AAV9, AAV9 K449R (or K449R AAV9), AAV1, AAVrhlO, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B 3 (PHP.B 3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2 Al 5/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9.16, AAV9.24, AAV9.45, AAV9.47, AAV9.61, AAV9.68, AAV9.84, AAV9.9, AAV10, AAV11, AAV 12, AAV16.3, AAV24.1, AAV27.3, AAV42.12, AAV42-lb, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAVl-7/rh.48, AAVl-8/rh.49, AAV2-15/rh.62, AAV2-3/rh.61, AAV2-4/rh.50, AAV2-5/rh.51, AAV3. l/hu.6, AAV3. l/hu.9, AAV3-9/rh.52, AAV3-1 l/rh.53, AAV4-8/rl 1.64, AAV4-9/rh.54, AAV4-l9/rh.55, AAV5-3/rh.57, AAV5-22/rh.58, AAV7.3/hu.7, AAVl6.8/hu.lO, AAVl6.l2/hu.l 1, AAV29.3/bb.l, AAV29.5/bb.2, AAVl06. l/hu.37, AAV1 l4.3/hu.40, AAVl27.2/hu.4l, AAVl27.5/hu.42, AAVl28.3/hu.44, AAVl30.4/hu.48, AAVl45. l/hu.53, AAVl45.5/hu.54, AAVl45.6/hu.55, AAVl6l. l0/hu.60, AAVl6l.6/hu.6l, AAV33. l2/hu.l7, AAV33.4/hu.l5, AAV33.8/hu.l6, AAV52/hu.l9, AAV52.l/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi. 1, AAVpi.3, AAVpi.2, AAVrh.60, AAVrh.44, AAVrh.65, AAVrh.55, AAVrh.47, AAVrh.69, AAVrh.45, AAVrh.59, AAVhu. 12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu. l, AAVhu.2, AAVhu.3, AAVhu.4, AAVhu.5, AAVhu.6, AAVhu.7, AAVhu.9, AAVhu.lO, AAVhu. l l, AAVhu. l3, AAVhu.l5, AAVhu.l6, AAVhu. l 7, AAVhu.l 8, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.4l, AAVhu.42, AAVhu.43, AAVhu.44, AAVhu.44Rl, AAVhu.44R2, AAVhu.44R3, AAVhu.45, AAVhu.46, AAVhu.47, AAVhu.48, AAVhu.48Rl, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.6l, AAVhu.63, AAVhu.64, AAVhu.66, AAVhu.67, AAVhu. l 4/9, AAVhu.t 19, AAVrh.2, AAVrh.2R, AAVrh.8, AAVrh.8R, AAVrh. lO, AAVrh.l2, AAVrh. l3, AAVrh.l3R, AAVrh. l4, AAVrh.l7, AAVrh. l8, AAVrh.l9, AAVrh.20, AAVrh.2l, AAVrh.22, AAVrh.23, AAVrh.24, AAVrh.25, AAVrh.3 l, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.l, AAVrh.48.l.2, AAVrh.48.2, AAVrh.49, AAVrh.5l, AAVrh.52, AAVrh.53, AAVrh.54, AAVrh.56, AAVrh.57, AAVrh.58, AAVrh.6l, AAVrh.64, AAVrh.64Rl, AAVrh.64R2, AAVrh.67, AAVrh.73, AAVrh.74, AAVrh8R, AAVrh8R A586R mutant, AAVrh8R R533 A mutant, AAAV, BAAV, caprine AAV, bovine AAV, AAVhEl.l, AAVhErl.5, AAVhERl. l4, AAVhErl.8, AAVhErl. l6, AAVhErl.l8, AAVhErl.35, AAVhErl.7, AAVhErl.36, AAVhEr2.29, AAVhEr2.4, AAVhEr2.l6, AAVhEr2.30, AAVhEr2.3 l, AAVhEr2.36, AAVhERl.23, AAVhEr3.l, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-lOl, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.l9, AAVhu. l l, AAVhu.53, AAV4-8/rh.64, AAVLG-9/hu.39, AAV54.5/hu.23, AAV54.2/hu.22, AAV54.7/hu.24, AAV54.1/hu.2l, AAV54.4R/hu.27, AAV46.2/hu.28, AAV46.6/hu.29, AAVl28. l/hu.43, true type AAV (ttAAV), EGRENN AAV 10, Japanese AAV 10 serotypes, AAV CBr-7.l, AAV CBr-7. lO, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-El, AAV CBr-E2, AAV CBr-E3, AAV CBr-E4, AAV CBr-E5, AAV CBr-e5, AAV CBr-E6, AAV CBr-E7, AAV CBr-E8, AAV CHt-l, AAV CHt-2, AAV CHt-3, AAV CHt-6. l, AAV CHt-6.lO, AAV CHt-6.5, AAV CHt-6.6, AAV CHt-6.7, AAV CHt-6.8, AAV CHt-Pl, AAV CHt-P2, AAV CHt-P5, AAV CHt-P6, AAV CHt-P8, AAV CHt-P9, AAV CKd-l, AAV CKd-lO, AAV CKd-2, AAV CKd-3, AAV CKd-4, AAV CKd-6, AAV CKd-7, AAV CKd-8, AAV CKd-Bl, AAV CKd-B2, AAV CKd-B3, AAV CKd-B4, AAV CKd-B5, AAV CKd-B6, AAV CKd-B7, AAV CKd-B8, AAV CKd-Hl, AAV CKd-H2, AAV CKd-H3, AAV CKd-H4, AAV CKd-H5, AAV CKd-H6, AAV CKd-N3, AAV CKd-N4, AAV CKd-N9, AAV CLg-Fl, AAV CLg-F2, AAV CLg-F3, AAV CLg-F4, AAV CLg-F5, AAV CLg-F6, AAV CLg-F7, AAV CLg-F8, AAV CLv-l, AAV CLvl-l, AAV Clvl-lO, AAV CLvl-2, AAV CLv-l2, AAV CLvl-3, AAV CLv-l 3, AAV CLvl-4, AAV Clvl-7, AAV Clvl-8, AAV Clvl-9, AAV CLv-2, AAV CLv-3, AAV CLv-4, AAV CLv-6, AAV CLv-8, AAV CLv-Dl, AAV CLv-D2, AAV CLv-D3, AAV CLv-D4, AAV CLv-D5, AAV CLv-D6, AAV CLv-D7, AAV CLv-D8, AAV CLy-El, AAV CLv-Kl, AAV CLv-K3, AAV CLv-K6, AAV CLv-L4, AAV CLv-L5, AAV CLv-L6, AAV CLv-Ml, AAV CLv-Ml l, AAV CLv-M2, AAV CLv-M5, AAV CLv-M6, AAV CLv-M7, AAV CLv-M8, AAV CLv-M9, AAV CLv-Rl, AAV CLv-R2, AAV CLv-R3, AAV CLv-R4, AAV CLv-R5, AAV CLv-R6, AAV CLv-R7, AAV CLv-R8, AAV CLv-R9, AAV CSp-l, AAV CSp-lO, AAV CSp-l 1, AAV CSp-2, AAV CSp-3, AAV CSp-4, AAV CSp-6, AAV CSp-7, AAV CSp-8, AAV CSp-8. l0, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, variants thereof, a hybrid or chimera of any of the foregoing AAV serotypes, or any combination thereof.

Disclosed herein include nucleic acids. In some embodiments, the nucleic acid comprises a sequence encoding an AAV acoustic targeting peptide disclosed herein. In some embodiments, the nucleic acid comprises a sequence encoding an AAV capsid protein disclosed herein.

Disclosed herein include recombinant adeno-associated virus (rAAV). In some embodiments, the rAAV comprises an AAV acoustic targeting peptide disclosed herein, or an AAV capsid protein disclosed herein. In some embodiments, the rAAV comprises an AAV capsid protein which comprises an AAV acoustic targeting peptide disclosed herein, wherein the amino acid sequence is inserted between two adjacent amino acids in AA586-592, or functional equivalents thereof, of the AAV capsid protein. In some embodiments, the two adjacent amino acids are AA588 and AA589. In some embodiments, the AAV capsid protein comprises, or consists thereof, SEQ ID NO: 6 (AAV.FUS.1), SEQ ID NO: 7 (AAV.FUS.2), SEQ ID NO: 8 (AAV.FUS.3), SEQ ID NO: 9 (AAV.FUS.4), and/or SEQ ID NO: 10 (AAV.FUS.5). In some embodiments, the rAAV comprises an rAAV vector genome.

Disclosed herein include compositions. In some embodiments, the composition comprises: an AAV acoustic targeting peptide disclosed herein, an AAV capsid protein disclosed herein, a nucleic acid disclosed herein, an rAAV disclosed herein, or a combination thereof. In some embodiments, the composition is a pharmaceutical composition comprising one or more pharmaceutical acceptable carriers.

Disclosed herein include compositions for use in the delivery of an agent to a target environment of a subject. In some embodiments, the composition comprises: an AAV comprising (1) an AAV capsid protein disclosed herein and (2) an agent to be delivered to the target environment of the subject. In some embodiments, the target environment comprises one or more target brain region(s).

The composition can be a pharmaceutical composition comprising one or more pharmaceutical acceptable carriers. In some embodiments, the agent to be delivered comprises a nucleic acid, a peptide, a small molecule, an aptamer, or a combination thereof. In some embodiments, the nucleic acid comprises one or more of: a) a DNA sequence that encodes a trophic factor, a growth factor, or a soluble protein; b) a cDNA that restores protein function to humans or animals harboring a genetic mutation(s) in that gene; c) a cDNA that encodes a protein that can be used to control or alter the activity or state of a cell; d) a cDNA that encodes a protein or a nucleic acid that can be used for assessing the state of a cell; e) a cDNA that encodes a protein for gene editing, or a guide RNA; f) a DNA sequence for genome editing via homologous recombination; g) a DNA sequence encoding a therapeutic RNA; h) an shRNA or an artificial miRNA delivery system; and i) a DNA sequence that influences the splicing of an endogenous gene.

In some embodiments, the nucleic acid encodes one or more payload proteins and/or one or more payload RNA agents (e.g., one or more components of a synthetic protein circuit). In some embodiments, the payload protein and/or the payload RNA agent is capable of diminishing the concentration, stability, and/or activity an endogenous protein. In some embodiments, the payload protein is a therapeutic protein or a variant thereof. In some embodiments, the therapeutic protein is configured to prevent or treat a disease or disorder of a subject, optionally the subject suffers from a deficiency of said therapeutic protein. In some embodiments, the payload RNA agent comprises one or more of dsRNA, siRNA, shRNA, pre-miRNA, pri-miRNA, miRNA, stRNA, lncRNA, piRNA, and snoRNA. In some embodiments, a payload protein comprises a chemogenetic protein. In some embodiments, the chemogenetic protein is selected from the comprising DREADD, PSAM, TrpV1, hM2Di, hM4Di, hM1Dq, hM3Dq, hM5Dq, or any combination thereof.

In some embodiments, the payload protein comprises fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof. In some embodiments, the payload protein comprises nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof.

The payload protein can comprise a diagnostic agent. In some embodiments, the diagnostic agent comprises green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof.

The payload protein can comprise a programmable nuclease. In some embodiments, the programmable nuclease is selected from the group comprising: SpCas9 or a derivative thereof; VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof; dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions. In some embodiments, the programmable nuclease comprises a zinc finger nuclease (ZFN) and/or transcription activator-like effector nuclease (TALEN). In some embodiments, the programmable nuclease comprises Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, Cas1, Cas1B, Cas2, Cas3, Cas4, Cas5, Cash, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpfl, C2c1, C2c3, Cas12a, Cas12b, Cas12c, Cas12d, Cas12e, Cas13a, Cas13b, Cas13c, derivatives thereof, or any combination thereof. In some embodiments, the nucleic acid further comprises a polynucleotide encoding (i) a targeting molecule and/or (ii) a donor nucleic acid. In some embodiments, the targeting molecule is capable of associating with the programmable nuclease. In some embodiments, the targeting molecule comprises single strand DNA or single strand RNA, optionally the targeting molecule comprises a single guide RNA (sgRNA).

In some embodiments, the nucleic acid comprises a tandem gene expression element selected from the group an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof. In some embodiments, the nucleic acid comprises a promoter operably linked to a polynucleotide encoding the one or more payload protein and/or the one or more payload RNA agents. In some embodiments, the promoter is capable of inducing the transcription of the polynucleotide, further optionally the nucleic acid comprises one or more of a 5′ UTR, 3′ UTR, a minipromoter, an enhancer, a splicing signal, a polyadenylation signal, a terminator, a protein degradation signal, and an internal ribosome-entry element (IRES). In some embodiments, the polynucleotide further comprises a transcript stabilization element. In some embodiments, the transcript stabilization element comprises woodchuck hepatitis post-translational regulatory element (WPRE), bovine growth hormone polyadenylation (bGH-polyA) signal sequence, human growth hormone polyadenylation (hGH-polyA) signal sequence, or any combination thereof.

In some embodiments, the promoter comprises a ubiquitous promoter. In some embodiments, the ubiquitous promoter is selected from the group comprising a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CBH promoter, or any combination thereof.

The promoter can be an inducible promoter, for example a tetracycline responsive promoter, a TRE promoter, a Tre3G promoter, an ecdysone responsive promoter, a cumate responsive promoter, a glucocorticoid responsive promoter, and estrogen responsive promoter, a PPAR-y promoter, or an RU-486 responsive promoter. In some embodiments, the promoter comprises a tissue-specific promoter, a lineage-specific promoter, and/or a promoter configured to be active in target brain cell(s) of a subject. In some embodiments, the tissue specific promoter is a neuron-specific promoter. In some embodiments, the neuron-specific promoter comprises a synapsin-1 (Syn) promoter, a CaMKIIa promoter, a calcium/calmodulin-dependent protein kinase II a promoter, a tubulin alpha I promoter, a neuron-specific enolase promoter, a platelet-derived growth factor beta chain promoter, TRPV1 promoter, a Nav1.7 promoter, a Nav1.8 promoter, a Nav1.9 promoter, or an Advillin promoter. In some embodiments, the composition is for intravenous administration and/or systemic administration.

Disclosed herein include methods of delivering an agent to one or more target brain region(s) of a subject. The method can comprise: providing an AAV vector comprising an AAV capsid protein disclosed herein, wherein the AAV vector further comprises an agent to be delivered to the target environment of the subject, optionally the target environment comprises one or more target brain region(s); and administering the AAV vector to the subject. In some embodiments, the method comprises: providing an AAV vector comprising an AAV capsid protein disclosed herein, wherein the AAV vector further comprises an agent to be delivered to the nervous system; and administering the AAV vector to the subject. In some embodiments, the administering comprises injection(s) into the one or more target brain region(s). In some embodiments, the method further comprises: administering to the subject an effective amount of a microbubble contrast agent; and applying focused ultrasound (FUS) to the one or more target brain region(s) of the subject. In some embodiments, the agent is delivered to target brain cell(s) of the target brain region(s) of the subject at least about 1.1-fold more efficiently than the delivery of the agent to peripheral organs of the subject (e.g., the liver). In some embodiments, the AAV vector is at least about 1.1-fold more efficient at crossing physically loosened biophysical barriers as compared to the corresponding parental AAV vector that does not comprise the acoustic targeting peptide.

Disclosed herein include methods of treating or preventing a disease or disorder in a subject in need thereof. In some embodiments, the method comprises: administering to the subject an effective amount of a composition disclosed herein (e.g., an AAV capsid protein disclosed herein and an agent to be delivered to the target environment of the subject). The method can comprise: administering to the subject an effective amount of a microbubble contrast agent. The method can comprise: applying focused ultrasound (FUS) to one or more target brain region(s) of the subject, thereby treating or preventing the disease or disorder in the subject.

Disclosed herein include methods of delivering an agent to one or more target brain region(s) of a subject. In some embodiments, the method comprises: administering to the subject an effective amount of a composition disclosed herein (e.g., an AAV capsid protein disclosed herein and an agent to be delivered to the target environment of the subject). The method can comprise: administering to the subject an effective amount of a microbubble contrast agent. The method can comprise: applying focused ultrasound (FUS) to one or more target brain region(s) of the subject, thereby delivering the agent to the target brain region(s).

Disclosed herein include methods of controlling a target brain cell activity with respect to a target neural circuit of a subject. In some embodiments, the method comprises: administering to the subject an effective amount of a composition disclosed herein (e.g., an AAV capsid protein disclosed herein and an agent to be delivered to the target environment of the subject), wherein the AAV comprises a nucleic acid encoding a chemogenetic protein under control of a promoter configured to be active in the target brain cell(s), and wherein the chemogenetic protein is configured to activate or inhibit the target brain cell activity following binding with a corresponding chemical actuator or metabolite thereof. The method can comprise: administering to the subject an effective amount of a microbubble contrast agent. The method can comprise: applying focused ultrasound (FUS) to one or more target brain region(s) of the subject comprising one or more target brain cell(s), thereby delivering the AAV to the target brain cell(s) to obtain chemogenetically treated target brain region(s) in which target brain cell(s) express the chemogenetic protein. The method can comprise: administering to the subject an effective amount of the corresponding chemical actuator to allow binding of the corresponding chemical actuator or a metabolite thereof with the chemogenetic protein in the target brain cell(s) of chemogenetically treated target brain region(s) to activate or inhibit the target brain cell activity.

Disclosed herein include methods of modifying a target behavior or physiological function of a subject associated with a target brain cell activity with respect to a neural circuit of the subject. In some embodiments, the method comprises: administering to the subject an effective amount of a composition disclosed herein (e.g., an AAV capsid protein disclosed herein and an agent to be delivered to the target environment of the subject), wherein the AAV comprises a nucleic acid encoding a chemogenetic protein under control of a promoter configured to be active in the target brain cell(s), and wherein the chemogenetic protein is configured to activate or inhibit the target brain cell activity following binding with a corresponding chemical actuator or metabolite thereof. The method can comprise: administering to the subject an effective amount of a microbubble contrast agent. The method can comprise: applying focused ultrasound (FUS) to one or more target brain region(s) of the subject comprising one or more target brain cell(s), thereby delivering the AAV to the target brain cell(s) to obtain chemogenetically treated target brain region(s) in which target brain cell(s) express the chemogenetic protein. The method can comprise: administering to the subject an effective amount of the corresponding chemical actuator to allow binding of the corresponding chemical actuator or a metabolite thereof with the chemogenetic protein in the target brain cell(s) of chemogenetically treated target brain region(s) to activate or inhibit the target brain cell activity and thereby modify the target behavior or physiological function of the subject.

Disclosed herein include methods of treating or preventing a disease or disorder in a subject associated with a target brain cell activity with respect to a neural circuit of the subject. In some embodiments, the method comprises: administering to the subject an effective amount of a composition disclosed herein (e.g., an AAV capsid protein disclosed herein and an agent to be delivered to the target environment of the subject), wherein the AAV comprises a nucleic acid encoding a chemogenetic protein under control of a promoter configured to be active in the target brain cell(s), and wherein the chemogenetic protein is configured to activate or inhibit the target brain cell activity following binding with a corresponding chemical actuator or metabolite thereof. The method can comprise: administering to the subject an effective amount of a microbubble contrast agent. The method can comprise: applying focused ultrasound (FUS) to one or more target brain region(s) of the subject comprising one or more target brain cell(s), thereby delivering the AAV to the target brain cell(s) to obtain chemogenetically treated target brain region(s) in which target brain cell(s) express the chemogenetic protein. The method can comprise: administering to the subject an effective amount of the corresponding chemical actuator to allow binding of the corresponding chemical actuator or a metabolite thereof with the chemogenetic protein in the target brain cell(s) of chemogenetically treated target brain region(s) to activate or inhibit the target brain cell activity and thereby treat or prevent the disease or disorder in the subject.

In some embodiments, the target brain region(s) comprise one or more target brain cell(s). In some embodiments, applying FUS induces transient blood-brain barrier opening(s) (BBBO) at the target brain region(s). In some embodiments, the applying focused ultrasound is performed at a frequency of 100 kHz to 100 MHz. In some embodiments, the applying focused ultrasound is performed at a frequency of 0.2 to 1.5 MHz. In some embodiments, the applying focused ultrasound is performed within an ultrasound having a mechanical index in a range between about 0.2 and 0.6. In some embodiments, applying FUS to one or more target brain region(s) of the subject comprises applying one or more FUS pulses to the one or more target brain region(s) over a duration of time. In some embodiments, the duration of time is about 48 hours, about 44 hours, about 40 hours, about 35 hours, about 30 hours, about 25 hours, 20 hours, 15 hours, 10 hours, about 8 hours, about 8 hours, 8 hours, about 7 hours, about 6 hours, about 5 hours, about 4 hours, about 3 hours, about 2 hours, about 1 hour, about 30 minutes, about 15 minutes, about 10 minutes, or about 5 minutes. In some embodiments, the one or more US pulses each have a pulse duration of about 1 hour, about 30 minutes, about 15 minutes, about 10 minutes, about 5 minutes, about 1 minute, about 1 second, or about 1 millisecond.

Applying FUS can induce permeability (blood-brain-barrier opening(s)) of the one or more target brain region(s), optionally for a duration of time after applying FUS of about 8 weeks, about 7 weeks, about 6 weeks, about 5 weeks, about 4 weeks, about 3 weeks, about 2 weeks, about 1 week, about 6 days, about 5 days, about 4 days, about 3 days, about 48 hours, about 44 hours, about 40 hours, about 35 hours, about 30 hours, about 25 hours, 20 hours, 15 hours, 10 hours, about 8 hours, about 8 hours, 8 hours, about 7 hours, about 6 hours, about 5 hours, about 4 hours, about 3 hours, about 2 hours, about 1 hour, about 30 minutes, about 15 minutes, about 10 minutes, or about 5 minutes.

In some embodiments, the microbubble contrast agent comprises an encapsulated gas and a shell of lipid or protein. In some embodiments, the encapsulated gas is perfluren or C3F8. In some embodiments, the microbubble contrast agent is selected from the group comprising nanobubbles, Definity®, Sonovue®, Optison®, and USphere®. In some embodiments, the microbubble contrast agent has an average diameter of between about 10 nm and 1000 nm or between about 1 and 5 microns, optionally the microbubble contrast agent is a nanobubble. In some embodiments, the microbubble contrast agent and the composition are co-administered.

The method can comprise: administering a chemical actuator to the subject. In some embodiments, administering of the chemical actuator is performed at least one week after the administrating of the composition and the applying of focused ultrasound. In some embodiments, the chemical actuator is selected from the group comprising salvinorin B, KORD Dreadd, deschloroclozapine, clozapine-N-oxide, clozapine, compound 21, and perlapine.

In some embodiments, the AAV is administered at a dose at least about 1.1-fold lower as compared to a comparable method wherein the corresponding parental AAV that does not comprise the acoustic targeting peptide is administered. In some embodiments, the FUS is administered at a dose at least about 1.1-fold lower as compared to a comparable method wherein the corresponding parental AAV that does not comprise the acoustic targeting peptide is administered. In some embodiments, the AAV demonstrates at least about 1.1-fold reduced transduction of cells of peripheral tissue(s) and/or an at least about 1.1-fold greater transduction of target brain region(s) as compared to the corresponding parental AAV that does not comprise the acoustic targeting peptide. In some embodiments, the AAV has an at least about 1.1-fold improvement in targeting efficiency as compared to the corresponding parental AAV that does not comprise the acoustic targeting peptide, wherein targeting efficiency is the ratio of the brain transduction efficiency to liver transduction efficiency relative to the corresponding parental AAV that does not comprise the acoustic targeting peptide. In some embodiments, the AAV demonstrate an at least about 1.1-fold increase in target brain cell(s) transduced per virus administered and/or an at least about 1.1-fold decrease in peripheral transduction per virus administered as compared to the corresponding parental AAV that does not comprise the acoustic targeting peptide. In some embodiments, the AAV demonstrates an at least about 1.1-fold greater transduction of one or more target brain region(s) (e.g., the cortex, striatum, thalamus, hippocampus, and/or midbrain) as compared to the corresponding parental AAV that does not comprise the acoustic targeting peptide.

In some embodiments, the administering comprises systemic administration. In some embodiments, the systemic administration is intravenous, intramuscular, intraperitoneal, or intraarticular. In some embodiments, administering comprises intrathecal administration, intracranial injection, aerosol delivery, nasal delivery, vaginal delivery, direct injection to any tissue in the body, intraventricular delivery, intraocular delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof.

In some embodiments, the target brain region(s) has a size in a range between 1 and 10 mm. In some embodiments, the target brain region(s) comprises the Lateral parabrachial nucleus, brainstem, Medulla oblongata, Medullary pyramids, Olivary body, Inferior olivary nucleus, Rostral ventrolateral medulla, Respiratory center, Dorsal respiratory group, Ventral respiratory group, Pre-Bötzinger complex, Botzinger complex, Paramedian reticular nucleus, Cuneate nucleus, Gracile nucleus, Intercalated nucleus, Area postrema, Medullary cranial nerve nuclei, Inferior salivatory nucleus, Nucleus ambiguus, Dorsal nucleus of vagus nerve, Hypoglossal nucleus, Solitary nucleus, Pons, Pontine nuclei, Pontine cranial nerve nuclei, chief or pontine nucleus of the trigeminal nerve sensory nucleus (V), Motor nucleus for the trigeminal nerve (V), Abducens nucleus (VI), Facial nerve nucleus (VII), vestibulocochlear nuclei (vestibular nuclei and cochlear nuclei) (VIII), Superior salivatory nucleus, Pontine tegmentum, Respiratory centers, Pneumotaxic center, Apneustic center, Pontine micturition center (Barrington's nucleus), Locus coeruleus, Pedunculopontine nucleus, Laterodorsal tegmental nucleus, Tegmental pontine reticular nucleus, Superior olivary complex, Paramedian pontine reticular formation, Cerebellar peduncles, Superior cerebellar peduncle, Middle cerebellar peduncle, Inferior cerebellar peduncle, Cerebellum, Cerebellar vermis, Cerebellar hemispheres, Anterior lobe, Posterior lobe, Flocculonodular lobe, Cerebellar nuclei, Fastigial nucleus, Interposed nucleus, Globose nucleus, Emboliform nucleus, Dentate nucleus, Tectum, Corpora quadrigemina, inferior colliculi, superior colliculi, Pretectum, Tegmentum, Periaqueductal gray, Parabrachial area, Medial parabrachial nucleus, Subparabrachial nucleus (Kölliker-Fuse nucleus), Rostral interstitial nucleus of medial longitudinal fasciculus, Midbrain reticular formation, Dorsal raphe nucleus, Red nucleus, Ventral tegmental area, Substantia nigra, Pars compacta, Pars reticulata, Interpeduncular nucleus, Cerebral peduncle, Crus cerebri, Mesencephalic cranial nerve nuclei, Oculomotor nucleus (III), Trochlear nucleus (IV), Mesencephalic duct (cerebral aqueduct, aqueduct of Sylvius), Pineal body, Habenular nucleim Stria medullares, Taenia thalami, Subcommissural organ, Thalamus, Anterior nuclear group, Anteroventral nucleus (aka ventral anterior nucleus), Anterodorsal nucleus, Anteromedial nucleus, Medial nuclear group, Medial dorsal nucleus, Midline nuclear group, Paratenial nucleus, Reuniens nucleus, Rhomboidal nucleus, Intralaminar nuclear group, Centromedial nucleus, Parafascicular nucleus, Paracentral nucleus, Central lateral nucleus, Central medial nucleus, Lateral nuclear group, Lateral dorsal nucleus, Lateral posterior nucleus, Pulvinar, Ventral nuclear group, Ventral anterior nucleus, Ventral lateral nucleus, Ventral posterior nucleus, Ventral posterior lateral nucleus, Ventral posterior medial nucleus, Metathalamus, Medial geniculate body, Lateral geniculate body, Thalamic reticular nucleus, Hypothalamus, limbic system, HPA axis, preoptic area, Medial preoptic nucleus, Suprachiasmatic nucleus, Paraventricular nucleus, Supraoptic nucleusm Anterior hypothalamic nucleus, Lateral preoptic nucleus, median preoptic nucleus, periventricular preoptic nucleus, Tuberal, Dorsomedial hypothalamic nucleus, Ventromedial nucleus, Arcuate nucleus, Lateral area, Tuberal part of Lateral nucleus, Lateral tuberal nuclei, Mammillary nuclei, Posterior nucleus, Lateral area, Optic chiasm, Subfornical organ, Periventricular nucleus, Pituitary stalk, Tuber cinereum, Tuberal nucleus, Tuberomammillary nucleus, Tuberal region, Mammillary bodies, Mammillary nucleus, Subthalamus, Subthalamic nucleus, Zona incerta, Pituitary gland, neurohypophysis, Pars intermedia, adenohypophysis, cerebral hemispheres, Corona radiata, Internal capsule, External capsule, Extreme capsule, Arcuate fasciculus, Uncinate fasciculus, Perforant Path, Hippocampus, Dentate gyms, Cornu ammonis, Cornu ammonis area 1, Cornu ammonis area 2, Cornu ammonis area 3, Cornu ammonis area 4, Amygdala, Central nucleus, Medial nucleus (accessory olfactory system), Cortical and basomedial nuclei, Lateral and basolateral nuclei, extended amygdala, Stria terminalis, Bed nucleus of the stria terminalis, Claustrum, Basal ganglia, Striatum, Dorsal striatum (aka neostriatum), Putamen, Caudate nucleus, Ventral striatum, Striatum, Nucleus accumbens, Olfactory tubercle, Globus pallidus, Subthalamic nucleus, Basal forebrain, Anterior perforated substance, Substantia innominata, Nucleus basalis, Diagonal band of Broca, Septal nuclei, Medial septal nuclei, Lamina terminalis, Vascular organ of lamina terminalis, Olfactory bulb, Piriform cortex, Anterior olfactory nucleus, Olfactory tract, Anterior commissure, Uncus, Cerebral cortex, Frontal lobe, Frontal cortex, Primary motor cortex, Supplementary motor cortex, Premotor cortex, Prefrontal cortex, frontopolar cortex, Orbitofrontal cortex, Dorsolateral prefrontal cortex, dorsomedial prefrontal cortex, ventrolateral prefrontal cortex, Superior frontal gyms, Middle frontal gyms, Inferior frontal gyms, Brodmann areas (4, 6, 8, 9, 10, 11, 12, 24, 25, 32, 33, 44, 45, 46, and/or 47), Parietal lobe, Parietal cortex, Primary somatosensory cortex (S1), Secondary somatosensory cortex (S2), Posterior parietal cortex, postcentral gyms, precuneus, Brodmann areas (1, 2, 3 (Primary somesthetic area), 5, 7, 23, 26, 29, 31, 39, and/or 40), Occipital lobe, Primary visual cortex (V1), V2, V3, V4, V5/MT, Lateral occipital gyms, Cuneus, Brodmann areas (17 (V1, primary visual cortex), 18, and/or 19), temporal lobe, Primary auditory cortex (A1), secondary auditory cortex (A2), Inferior temporal cortex, Posterior inferior temporal cortex, Superior temporal gyms, Middle temporal gyms, Inferior temporal gyms, Entorhinal Cortex, Perirhinal Cortex, Parahippocampal gyms, Fusiform gyms, Brodmann areas (9, 20, 21, 22, 27, 34, 35, 36, 37, 38, 41, and/or 42), Medial superior temporal area (MST), insular cortex, cingulate cortex, Anterior cingulate, Posterior cingulate, dorsal cingulate, Retrosplenial cortex, Indusium griseum, Subgenual area 25, Brodmann areas (23, 24; 26, 29, 30 (retrosplenial areas), 31, and/or 32), cranial nerves (Olfactory (I), Optic (II), Oculomotor (III), Trochlear (IV), Trigeminal (V), Abducens (VI), Facial (VII), Vestibulocochlear (VIII), Glossopharyngeal (IX), Vagus (X), Accessory (XI), Hypoglossal (XII)), or any combination thereof. In some embodiments, the brain region comprises neural pathways Superior longitudinal fasciculus, Arcuate fasciculus, Thalamocortical radiations, Cerebral peduncle, Corpus callosum, Posterior commissure, Pyramidal or corticospinal tract, Medial longitudinal fasciculus, dopamine system, Mesocortical pathway, Mesolimbic pathway, Nigrostriatal pathway, Tuberoinfundibular pathway, serotonin system, Norepinephrine Pathways, Posterior column-medial lemniscus pathway, Spinothalamic tract, Lateral spinothalamic tract, Anterior spinothalamic tract, or any combination thereof.

The subject can be a subject suffering from or at a risk to develop one or more of chronic pain, Friedreich's ataxia, Huntington's disease (HD), Alzheimer's disease (AD), Parkinson's disease (PD), Amyotrophic lateral sclerosis (ALS), spinal muscular atrophy types I and II (SMA I and II), Friedreich's Ataxia (FA), Spinocerebellar ataxia, multiple sclerosis (MS), chronic traumatic encephalopathy (CTE), HIV-1 associated dementia, or lysosomal storage disorders that involve cells within the CNS. In some embodiments, the lysosomal storage disorder is Krabbe disease, Sandhoff disease, Tay-Sachs, Gaucher disease (Type I, II or III), Niemann-Pick disease (NPC1 or NPC2 deficiency), Hurler syndrome, Pompe Disease, or Batten disease In some embodiments, the subject is a subject suffering from, at risk to develop, or has suffered from a stroke, traumatic brain injury, epilepsy, or spinal cord injury. In some embodiments, the disease or disorder is a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof.

The disease or disorder can comprise a neurological disease or disorder. In some embodiments, the neurological disease or disorder comprises epilepsy, Dravet Syndrome, Lennox Gastaut Syndrome, mycolonic seizures, juvenile mycolonic epilepsy, refractory epilepsy, schizophrenia, juvenile spasms, West syndrome, infantile spasms, refractory infantile spasms, Alzheimer's disease, Creutzfeld-Jakob's syndrome/disease, bovine spongiform encephalopathy (BSE), prion related infections, diseases involving mitochondrial dysfunction, diseases involving β-amyloid and/or tauopathy, Down's syndrome, hepatic encephalopathy, Huntington's disease, motor neuron diseases, amyotrophic lateral sclerosis (ALS), olivoponto-cerebellar atrophy, post-operative cognitive deficit (POCD), systemic lupus erythematosus, systemic clerosis, Sjogren's syndrome, Neuronal Ceroid Lipofuscinosis, neurodegenerative cerebellar ataxias, Parkinson's disease, Parkinson's dementia, mild cognitive impairment, cognitive deficits in various forms of mild cognitive impairment, cognitive deficits in various forms of dementia, dementia pugilistica, vascular and frontal lobe dementia, cognitive impairment, learning impairment, eye injuries, eye diseases, eye disorders, glaucoma, retinopathy, macular degeneration, head or brain or spinal cord injuries, head or brain or spinal cord trauma, convulsions, epileptic convulsions, epilepsy, temporal lobe epilepsy, myoclonic epilepsy, tinnitus, dyskinesias, chorea, Huntington's chorea, athetosis, dystonia, stereotypy, ballism, tardive dyskinesias, tic disorder, torticollis spasmodicus, blepharospasm, focal and generalized dystonia, nystagmus, hereditary cerebellar ataxias, corticobasal degeneration, tremor, essential tremor, addiction, anxiety disorders, panic disorders, social anxiety disorder (SAD), attention deficit hyperactivity disorder (ADHD), attention deficit syndrome (ADS), restless leg syndrome (RLS), hyperactivity in children, autism, dementia, dementia in Alzheimer's disease, dementia in Korsakoff syndrome, Korsakoff syndrome, vascular dementia, dementia related to HIV infections, HIV-1 encephalopathy, AIDS encephalopathy, AIDS dementia complex, AIDS-related dementia, major depressive disorder, major depression, depression, memory loss, stress, bipolar manic-depressive disorder, drug tolerance, drug tolerance to opioids, movement disorders, fragile-X syndrome, irritable bowel syndrome (IBS), migraine, multiple sclerosis (MS), muscle spasms, pain, chronic pain, acute pain, inflammatory pain, neuropathic pain, posttraumatic stress disorder (PTSD), schizophrenia, spasticity, Tourette's syndrome, eating disorders, food addiction, binge eating disorders, agoraphobia, generalized anxiety disorder, obsessive-compulsive disorder, panic disorder, social phobia, phobic disorders, substance-induced anxiety disorder, delusional disorder, schizoaffective disorder, schizophreniform disorder, substance-induced psychotic disorder, hypertension, or any combination thereof.

### BRIEF DESCRIPTION OF THE DRAWINGS

FIGS. 1A-1B depict screening methodology for generation of an AAV for improved site-specific noninvasive gene delivery to the brain. (FIG. 1A) Summary of the high-throughput screening and selection process. AAV library is administered intravenously (IV.) and delivered to one brain hemisphere through FUS-BBBO. After 14 days mice are euthanized, their brain harvested, and the DNA from selected hemispheres is extracted. The DNA is then amplified by Cre-dependent PCR that enriches the viral DNA modified by Cre. In this study, neurons expressed Cre exclusively, and the Cre-dependent PCR enriched viral DNA of AAVs that transduced neurons. The obtained viral DNA was subjected to next-generation sequencing for the targeted hemisphere (round 1) or both targeted and control hemispheres (round 2). The process is then repeated for the next round (steps exclusive to round 2 indicated by the grey text). (FIG. 1B) Overall, 1.3 billion clones were screened in the first round, and 2098 clones in the second round of selection. Out of these clones, 5 were selected that were tested in low-throughput to yield AAV.FUS.3— a vector with enhanced FUS-BBBO gene delivery.

FIGS. 2A-2B depict data related to high throughput screening yielding vectors with improved FUS-BBBO gene delivery. (FIG. 2A) An MM image showing mouse brain with 4 sites opened with FUS-BBBO in one hemisphere. The bright areas (arrowheads) indicate successful BBB opening and extravasation of the MRI contrast agent Prohance into the brain. This BBB opening was used for delivery of the AAV library. (FIG. 2B) Sequencing results of round 2 of screening show a fraction of NGS reads within the DNA extracted from brains of Syn1-Cre mice subjected to FUS-BBBO and injected with a focused library of 2098 clones. Each dot represents a unique capsid protein sequence, and the position on each axis corresponds to the number of times the sequence was detected in the FUS-targeted and untargeted hemispheres. Markers below the dotted line represent sequences that on average showed 100-fold higher enrichment in the targeted hemisphere as compared to the control hemisphere. Dark grey dots represent 35 clones that are enriched in the FUS targeted hemispheres at least 100-fold in every tested mouse and DNA sequence encoding the 7-mer insertion peptide. Yellow dots represent 5 clones (AAV.FUS.1-5) selected for low-throughput testing. Due to the use of a logarithmic plot, clones that had zero copies detected in either of the hemispheres are not shown.

FIGS. 3A-3F depict data related to AAV.FUS candidates improving efficiency of gene delivery to the brain and reduce peripheral transduction. (FIG. 3A) Representative images were obtained from mice co-injected with AAV9 and a AAV.FUS.3 at 1010 viral particles per gram of body weight. After 3 weeks, the mice were perfused, brains were extracted and then sectioned at 50 microns. Sections were imaged on a confocal microscope with 20× objective showing brain transduction by AAV9 (red) and AAV.FUS.3 (green), and counterstained with a neuronal stain (NeuN, blue). (FIG. 3B) All but one (AAV.FUS.4) AAV.FUS candidates showed significant improvement over the co-injected AAV9. (FIG. 3C) It was found that few cells were transduced outside of the FUS-targeted site and AAV.FUS.3 and AAV9 were not significantly different (0.19% off target transduction for AAV.FUS.3 vs 0.4% for AAV9; p=0.072, two way Anova with Sidak multiple comparisons test). Similarly other candidates also showed no differences (AAV.FUS.1, p=0.99 n=6; AAV.FUS.2, p=0.98, n=5; AAV.FUS.4, p=0.86, n=6; AAV.FUS.5, p=0.83, n=6). (FIG. 3D) Representative images showing liver transduction by AAV9 (red) and AAV.FUS.3 (green). (FIG. 3E) All tested candidates showed reduction of the liver transduction as compared to the co-injected AAV9 in the same mice for which brain expression was analyzed. (FIG. 3F) The fold-improvement in targeting efficiency was defined as the ratio of brain transduction to the liver transduction efficiency using AAV9 as a baseline, which suggested that AAV.FUS.3 is the top candidate for further study. Scale bars are 50 microns in panels a, c, unless otherwise noted. (****=p<0.0001; ***=p<0.001; **=p<0.01; *=p<0.05, ns=not significant); Error bars are 95% CI.

FIGS. 4A-4C depict data related to AAV.FUS candidates showing improved neuronal tropism. (FIG. 4A) All AAV.FUS candidates show improved neuronal tropism (p<0.0001). Upon FUS-BBBO gene delivery, AAV.FUS.3. has 56% more likelihood of transducing a neuron than AAV9 (69.8%, vs 44.9% neuronal transduction, respectively; p<0.0001). (FIG. 4B) Representative images showing AAV9 transducing both neurons (blue, NeuN staining, example neuron designated by an arrow) and non-neuronal cells (example non-neuronal cell designated by an arrowhead), (FIG. 4C) In comparison, more of the cells transduced with AAV.FUS (green) are neurons (example neuron designated by an arrow), rather than non-neuronal cells (example cell designated by an arrowhead). Scale bars are 50 microns. (All p-values−****=p<0.0001). Error bars are 95% CI.

FIGS. 5A-5F depict data related to AAV.FUS.3 showing regional dependence of transduction efficiency. Hippocampus showed the highest, 4.3-fold, improvement in transduction over AAV9. (FIG. 5A) Representative image comparing transduction of the cortex with AAV.FUS.3 (green) and AAV9 (red). (FIG. 5B) Representative image comparing transduction of the striatum with AAV.FUS.3 (green) and AAV9 (red). (FIG. 5C) Representative image comparing transduction of the thalamus with AAV.FUS.3 (green) and AAV9 (red). (FIG. 5D) Representative image comparing transduction of the hippocampus with AAV.FUS.3 (green) and AAV9 (red). (FIG. 5E) Representative image comparing transduction of the midbrain with AAV.FUS.3 (green) and AAV9 (red). (FIG. 5F) AAV.FUS.3 shows regional differences in transduction efficiency of the tested regions—cortext (ctx), striatum (Str), thalamus (Th), hippocampus (Hpc), midbrain (Mb). All differences were statistically significant (one way ANOVA, “F (4, 10)=283.4”, P<0.0001; All pairwise comparison p-values <0.01, Tukey HSD post-hoc test). Scale bars are 50 microns. Error bars are 95% CI.

FIGS. 6A-6B depict non-limiting exemplary schematics related to construction of the AAV library and CRE-dependent PCR. (FIG. 6A) Randomized 21-basepair DNA fragment was inserted into the AAV9 capsid between amino acids 588 and 589, which resides at the exterior of an AAV capsid (inset). AAV capsid was produced within the AAV genome allowing for recovery of the capsid sequenced from transduced cells. The capsid coding sequence was followed by a polyA (pA) sequence flanked by a double-inverted floxed open reading frame (DIO). (FIG. 6B) The DIO sequence can be recombined and inverted in the presence of Cre enzyme. That sequence inversion can then be detected using PCR. Therefore, the DNA from AAVs that transduced cells expressing Cre can be amplified using a PCR reaction. In this study, hSyn1-Cre mice was used which express Cre selectively in neurons and thus selecting for neuron-transducing AAVs.

FIG. 7 depicts data related to AAV9 GFP+AAV9 mCherry. Transduced cell counts comparing AAV9 carrying GFP and mCherry are highly correlated and (Rsq=0.99). The mean numbers of transduced cells are not significantly different (fold difference between AAV9-GFP and AAV9-mCherry: 1.07-fold, p=0.081 (ns), paired t-test, 6 sections tested from 2 mice).

FIG. 8 depicts data related to pairwise comparison of AAV.FUS candidates transduction of the brain. Non-significant comparisons not shown for clarity. (****=p<0.0001; ***=p<0.001; **=p<0.01; *=p<0.05; F(4, 24)=14.96, P<0.0001, One-way ANOVA with Tukey HSD post hoc test.). Non-significant pairwise comparisons not shown for clarity. Error bars are 95% CI.

FIG. 9 depicts data related to pairwise comparison of AAV.FUS candidates transduction of the liver. Non-significant comparisons not shown for clarity. AAV.FUS.3 shows significantly reduced liver transduction compared to other AAV.FUS candidates. One-way ANOVA with Tukey HSD post-hoc test. F (4, 24)=96.69. P<0.0001; All pairwise comparisons are below p<0.0001, except AAV.FUS.2 vs AAV.FUS.4 (p=0.0001), AAV.FUS.2 vs AAV.FUS.5 (p=0.3524), AAV.FUS.3 vs AAV.FUS.4 (p=0.01), and AAV.FUS.4 vs AAV.FUS.5 (p=0.0099)). (****=p<0.0001; ***=p<0.001; **=p<0.01; *=p<0.05, ns=non significant). Error bars are 95% CI.

FIG. 10 depicts data related to representative images of transduction in brain and liver for all AAV.FUS (green, EGFP) and corresponding co-injected AAV9 control (red, mCherry) vectors. Scale bars, 200 microns for the brain, 100 microns for the liver.

FIG. 11 depicts data related to detailed pairwise comparisons for analysis of regional dependence of transduction efficiency for AAV.FUS.3. (****=p<0.0001; ***=p<0.001; **=p<0.01; *=p<0.05, ns=non significant; One-way ANOVA with Tukey HSD post-hoc test.).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include adeno-associated virus (AAV) acoustic targeting peptides. In some embodiments, the adeno-associated virus (AAV) acoustic targeting peptide comprises an amino acid sequence that comprises at least 4 contiguous amino acids from a sequence selected from the group consisting of AGNTSDR (SEQ ID NO: 1; AAV.FUS.1), ATDAYNK (SEQ ID NO: 2; AAV.FUS.2), WSEGGQP (SEQ ID NO: 3; AAV.FUS.3), SVGSADP (SEQ ID NO: 4; AAV.FUS.4), and VRMEGEV (SEQ ID NO: 5; AAV.FUS.5).

Disclosed herein include adeno-associated virus (AAV) capsid proteins. In some embodiments, the adeno-associated virus (AAV) capsid protein comprises an AAV acoustic targeting peptide disclosed herein

Disclosed herein include nucleic acids. In some embodiments, the nucleic acid comprises a sequence encoding an AAV acoustic targeting peptide disclosed herein. In some embodiments, the nucleic acid comprises a sequence encoding an AAV capsid protein disclosed herein.

Disclosed herein include recombinant adeno-associated virus (rAAV). In some embodiments, the rAAV comprises an AAV acoustic targeting peptide disclosed herein, or an AAV capsid protein disclosed herein.

In some embodiments, the rAAV comprises an AAV capsid protein which comprises an AAV acoustic targeting peptide disclosed herein, wherein the amino acid sequence is inserted between two adjacent amino acids in AA586-592, or functional equivalents thereof, of the AAV capsid protein. In some embodiments, the two adjacent amino acids are AA588 and AA589.

Disclosed herein include compositions. In some embodiments, the composition comprises: an AAV acoustic targeting peptide disclosed herein, an AAV capsid protein disclosed herein, a nucleic acid disclosed herein, an rAAV disclosed herein, or a combination thereof. In some embodiments, the composition is a pharmaceutical composition comprising one or more pharmaceutical acceptable carriers.

Disclosed herein include compositions for use in the delivery of an agent to a target environment of a subject. In some embodiments, the composition comprises: an AAV comprising (1) an AAV capsid protein disclosed herein and (2) an agent to be delivered to the target environment of the subject. In some embodiments, the target environment comprises one or more target brain region(s).

Disclosed herein include methods of delivering an agent to one or more target brain region(s) of a subject. In some embodiments, the method comprises: providing an AAV vector comprising an AAV capsid protein disclosed herein, wherein the AAV vector further comprises an agent to be delivered to the target environment of the subject, optionally the target environment comprises one or more target brain region(s); and administering the AAV vector to the subject. In some embodiments, the method comprises: providing an AAV vector comprising an AAV capsid protein disclosed herein, wherein the AAV vector further comprises an agent to be delivered to the nervous system; and administering the AAV vector to the subject.

Disclosed herein include methods of treating or preventing a disease or disorder in a subject in need thereof. In some embodiments, the method comprises: administering to the subject an effective amount of a composition disclosed herein (e.g., an AAV capsid protein disclosed herein and an agent to be delivered to the target environment of the subject). The method can comprise: administering to the subject an effective amount of a microbubble contrast agent. The method can comprise: applying focused ultrasound (FUS) to one or more target brain region(s) of the subject, thereby treating or preventing the disease or disorder in the subject.

Disclosed herein include methods of delivering an agent to one or more target brain region(s) of a subject. In some embodiments, the method comprises: administering to the subject an effective amount of a composition disclosed herein (e.g., an AAV capsid protein disclosed herein and an agent to be delivered to the target environment of the subject). The method can comprise: administering to the subject an effective amount of a microbubble contrast agent. The method can comprise: applying focused ultrasound (FUS) to one or more target brain region(s) of the subject, thereby delivering the agent to the target brain region(s).

Disclosed herein include methods of controlling a target brain cell activity with respect to a target neural circuit of a subject. In some embodiments, the method comprises: administering to the subject an effective amount of a composition disclosed herein (e.g., an AAV capsid protein disclosed herein and an agent to be delivered to the target environment of the subject), wherein the AAV comprises a nucleic acid encoding a chemogenetic protein under control of a promoter configured to be active in the target brain cell(s), and wherein the chemogenetic protein is configured to activate or inhibit the target brain cell activity following binding with a corresponding chemical actuator or metabolite thereof. The method can comprise: administering to the subject an effective amount of a microbubble contrast agent. The method can comprise: applying focused ultrasound (FUS) to one or more target brain region(s) of the subject comprising one or more target brain cell(s), thereby delivering the AAV to the target brain cell(s) to obtain chemogenetically treated target brain region(s) in which target brain cell(s) express the chemogenetic protein. The method can comprise: administering to the subject an effective amount of the corresponding chemical actuator to allow binding of the corresponding chemical actuator or a metabolite thereof with the chemogenetic protein in the target brain cell(s) of chemogenetically treated target brain region(s) to activate or inhibit the target brain cell activity.

Disclosed herein include methods of modifying a target behavior or physiological function of a subject associated with a target brain cell activity with respect to a neural circuit of the subject. In some embodiments, the method comprises: administering to the subject an effective amount of a composition disclosed herein (e.g., an AAV capsid protein disclosed herein and an agent to be delivered to the target environment of the subject), wherein the AAV comprises a nucleic acid encoding a chemogenetic protein under control of a promoter configured to be active in the target brain cell(s), and wherein the chemogenetic protein is configured to activate or inhibit the target brain cell activity following binding with a corresponding chemical actuator or metabolite thereof. The method can comprise: administering to the subject an effective amount of a microbubble contrast agent. The method can comprise: applying focused ultrasound (FUS) to one or more target brain region(s) of the subject comprising one or more target brain cell(s), thereby delivering the AAV to the target brain cell(s) to obtain chemogenetically treated target brain region(s) in which target brain cell(s) express the chemogenetic protein. The method can comprise: administering to the subject an effective amount of the corresponding chemical actuator to allow binding of the corresponding chemical actuator or a metabolite thereof with the chemogenetic protein in the target brain cell(s) of chemogenetically treated target brain region(s) to activate or inhibit the target brain cell activity and thereby modify the target behavior or physiological function of the subject.

Disclosed herein include methods of treating or preventing a disease or disorder in a subject associated with a target brain cell activity with respect to a neural circuit of the subject. In some embodiments, the method comprises: administering to the subject an effective amount of a composition disclosed herein (e.g., an AAV capsid protein disclosed herein and an agent to be delivered to the target environment of the subject), wherein the AAV comprises a nucleic acid encoding a chemogenetic protein under control of a promoter configured to be active in the target brain cell(s), and wherein the chemogenetic protein is configured to activate or inhibit the target brain cell activity following binding with a corresponding chemical actuator or metabolite thereof. The method can comprise: administering to the subject an effective amount of a microbubble contrast agent. The method can comprise: applying focused ultrasound (FUS) to one or more target brain region(s) of the subject comprising one or more target brain cell(s), thereby delivering the AAV to the target brain cell(s) to obtain chemogenetically treated target brain region(s) in which target brain cell(s) express the chemogenetic protein. The method can comprise: administering to the subject an effective amount of the corresponding chemical actuator to allow binding of the corresponding chemical actuator or a metabolite thereof with the chemogenetic protein in the target brain cell(s) of chemogenetically treated target brain region(s) to activate or inhibit the target brain cell activity and thereby treat or prevent the disease or disorder in the subject.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present disclosure, the following terms are defined below.

The term “brain cell” as used herein shall be given its ordinary meaning and shall also refer to cells that form the brain of an individual with the exclusion of blood vessels and meninges (dura mater, arachnoid mater, and pia mater in mammals) of the individual. Exemplary brain cells comprise neurons and glial cells.

The terms “neuron”, “nerve cell or “neural cell” as used herein interchangeably shall be given their ordinary meaning and also indicate an electrically excitable cell that receives, processes, and transmits information through electrical and chemical signals. A neuron consists of a cell body (or soma) which contains the neuron's nucleus (with DNA and typical nuclear organelles), branching dendrites (signal receivers) and a projection called axon, which take information away from the cell body and conduct the nerve signal. At the other end of the axon, axon terminals transmit the electro-chemical signal across a synapse (the gap between the axon terminal and the receiving cell). Accordingly, neural brain cells are nerve cells of the brain that transmit nerve signals to and from the brain.

Brain cells are comprised within areas of the brain defined as gray matter and white matter. The gray matter indicates an area of the brain comprising primarily neuronal cell bodies, neuropil (dendrites and myelinated as well as unmyelinated axons), glial cells (astrocytes and microglia), and synapses. White matter indicates an area of the brain which mainly comprise myelinated axons, also called tracts.

The wording “glial cells” as used herein shall be given its ordinary meaning and shall also refer to non-neuronal cells that maintain homeostasis, form myelin, and provide support and protection for neurons within the gray matter of the brain. Glial cells typically comprise macroglial cell such as oligodendrocytes, astrocytes, and ependymal cells, and microglia. Oligodendrocytes are cells that coat axons in the central nervous system (CNS) with their cell membrane, forming a specialized membrane differentiation called myelin, producing the myelin sheath. The myelin sheath provides insulation to the axon that allows electrical signals to propagate more efficiently Astrocytes (also called astroglia) are cells having numerous projections that link neurons to their blood supply while forming the blood-brain barrier. Astrocytes regulate the external chemical environment of neurons by removing excess potassium ions, and recycling neurotransmitters released during synaptic transmission. In some embodiments, astrocytes in the gray matter of a brain comprise protoplasmic astrocytes having short, thick, highly branched processes. Ependymal cells, also named ependymocytes, line the ventricular system of the brain and are involved in the creation and secretion of cerebrospinal fluid (CSF) and beat their cilia to help circulate the CSF and make up the blood-CSF barrier and are also thought to act as neural stem cells. Microglia includes specialized macrophages capable of phagocytosis that protect neurons of the central nervous system.

Brain cells are also comprised within “brain regions” which are areas anatomically defined by appearance and position as well as by their locations and their relationships with other parts of the brain. Exemplary brain regions can comprise the medulla (region containing many small nuclei involved in a wide variety of sensory and involuntary motor functions such as vomiting, heart rate and digestive processes), the pons (region of the brainstem directly above the medulla, which contains nuclei that control often voluntary but simple acts such as sleep, respiration, swallowing, bladder function, equilibrium, eye movement, facial expressions, and posture, includes) the hypothalamus (small region at the base of the forebrain composed of numerous small nuclei, each with distinct connections and neurochemistry, and engaged in additional involuntary or partially voluntary acts such as sleep and wake cycles, eating and drinking, and the release of some hormones), the thalamus (a region of nuclei with diverse functions such as relaying information to and from the cerebral hemispheres, motivation, and action-generating systems such as the action generating systems for several types of “consummatory” behaviors such as eating, drinking, defecation, and copulation, in the subthalamic area also zona incerta), the cerebellum (a region modulating the outputs of other brain regions, whether motor related or thought related, to make them certain and precise), the optic tectum (a region usually referred to as the superior colliculus in mammals, allowing actions such as eye movements and reaching movements to be directed toward points in space, most commonly in response to visual input), the pallium (a region of gray matter that lies on the surface of the forebrain also identified in reptiles and mammals as cerebral cortex which with multiple functions including smell and spatial memory), the hippocampus, (a region involved in complex events such as spatial memory and navigation in fishes, birds, reptiles, and mammals), the basal ganglia (a region involved in action selection as the related brain cells send inhibitory signals to all parts of the brain that can generate motor behaviors, and in the right circumstances release the inhibition, it comprises regions such as caudate nucleus, putamen, globus pallidus, substantia nigra, subthalamic nucleus, nucleus accumbens) and the olfactory bulb (a region that processes olfactory sensory signals and sends its output to the olfactory part of the pallium.

In some embodiments brain cells are further comprised in neural circuits possibly comprising cells and regions of additional parts of the body including cells of the peripheral nervous systems and other systems and organs of the body of the individual.

The wording “neural circuits” shall be given its ordinary meaning and shall also refer to a population of cells including neurons interconnected by synapses to pass an electrochemical signal from a neuron to another to carry out a specific function when activated. In some embodiments, the specific function neural circuits herein described manifests in a behavior or physiological function of the individual.

The term “behavior” as used herein shall be given its ordinary meaning and shall also refer to an internally coordinated responses (actions or inactions) of a whole living individual to internal and/or external stimuli. Exemplary behaviors in the sense of the disclosure comprise eating, drinking, defecation, and copulation, speaking, contemplating, remembering, focusing attention and additional behaviors identifiable by a skilled person.

The wording “physiological function” as used herein shall be given its ordinary meaning and shall also refer to a series of action and reactions performed by components of a living organism such as organ systems, organs, cells, and biomolecules to carry out the chemical and physical functions that exist in the living system. Exemplary physiological functions comprise action and reactions performed by components of the organism of an individual to carry out digestion of food, circulation of blood, contraction of muscles as well as other biophysical and biochemical phenomena, related to the coordinated homeostatic control mechanisms, and the continuous communication between cells in a living organism.

Neural circuits control behaviors and physiological function of an individual and changes in activity of neural circuits can lead to changes in behaviors and physiological functions of an individual as will be understood by a skilled person.

Exemplary neural circuit comprise the trisynaptic circuit in the hippocampus. the Papez circuit linking the hypothalamus to the limbic lobe, and neural circuits in the cortico-basal ganglia-thalamo-cortical loop which transmit information from the cortex, to basal ganglia, and thalamus, and back to the cortex, as well as the microcircuitry internal to the striatum the largest structure within the basal ganglia and additional circuits identifiable by a skilled person.

Methods and systems of the disclosure and related vectors and compositions in some embodiments only target brain cells whose cell bodies, dendrites or synapses are located in the gray matter. Accordingly in some embodiments “target brain cell” refers only to brain cells of the gray matter and the wording “target brain regions” only refer to brain regions comprising target brain cells, such as cerebral cortex, cerebellum, thalamus; hypothalamus; subthalamus, basal ganglia such as putamen, globus pallidus, nucleus accumbens; septal nuclei, deep cerebellar nuclei, dentate nucleus, globose nucleus, emboliform nucleus, fastigial nucleus), brainstem and regions thereof such as sub stantia nigra, red nucleus, olivary nuclei, cranial nerve nuclei. The wording “target neural circuit” as used herein can refer to neural circuits that comprise target brain cells such as trisynaptic circuit in the hippocampus.

As used herein, the terms “nucleic acid” and “polynucleotide” are interchangeable and can refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

The term “vector” as used herein, can refer to a vehicle for carrying or transferring a nucleic acid. Non-limiting examples of vectors include plasmids and viruses (for example, AAV viruses).

The term “construct,” as used herein, can refer to a recombinant nucleic acid that has been generated for the purpose of the expression of a specific nucleotide sequence(s), or that is to be used in the construction of other recombinant nucleotide sequences.

As used herein, the term “plasmid” can refer to a nucleic acid that can be used to replicate recombinant DNA sequences within a host organism. The sequence can be a double stranded DNA.

The term “virus genome” refers to a nucleic acid sequence that is flanked by cis acting nucleic acid sequences that mediate the packaging of the nucleic acid into a viral capsid. For AAVs and parvoviruses, for example it is known that the “inverted terminal repeats” (ITRs) that are located at the 5′ and 3′ end of the viral genome have this function and that the ITRs can mediate the packaging of heterologous, for example, non-wildtype virus genomes, into a viral capsid.

The term “element” can refer to a separate or distinct part of something, for example, a nucleic acid sequence with a separate function within a longer nucleic acid sequence. The term “regulatory element” and “expression control element” are used interchangeably herein and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and a ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “enhancer” refers to a type of regulatory element that can increase the efficiency of transcription, regardless of the distance or orientation of the enhancer relative to the start site of transcription.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

The term “construct,” as used herein, can refer to a recombinant nucleic acid that has been generated for the purpose of the expression of a specific nucleotide sequence(s), or that is to be used in the construction of other recombinant nucleotide sequences.

As used herein, the term “variant” can refer to a polynucleotide or polypeptide having a sequence substantially similar to a reference polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polypeptide as determined by sequence alignment programs known by skilled artisans.

The term “AAV” or “adeno-associated virus” refers to a Dependoparvovirus within the Parvoviridae genus of viruses. For example, the AAV can be an AAV derived from a naturally occurring “wild-type” virus, an AAV derived from a rAAV genome packaged into a capsid derived from capsid proteins encoded by a naturally occurring cap gene and/or a rAAV genome packaged into a capsid derived from capsid proteins encoded by a non-natural capsid cap gene, for example, XL.D1c-AAV9 and XL.N1-AAV9. Non-limited examples of AAV include AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 11 (AAV11), AAV type 12 (AAV12), AAV type DJ (AAV-DJ), avian AAV, bovine AAV, canine AAV, equine AAV, primate AAV, non-primate AAV, and ovine AAV. In some instances, the AAV is described as a “Primate AAV,” which refers to AAV that infect primates. Likewise an AAV may infect bovine animals (e.g., “bovine AAV”, and the like). In some instances, the AAV is wild type, or naturally occurring. In some instances the AAV is recombinant.

The term “AAV capsid” as used herein refers to a capsid protein or peptide of an adeno-associated virus. In some instances, the AAV capsid protein is configured to encapsidate genetic information (e.g., a heterologous nucleic acid, a transgene, therapeutic nucleic acid, viral genome). In some instances, the AAV capsid of the instant disclosure is a variant AAV capsid, which means in some instances that it is a parental or wild-type AAV capsid that has been modified in an amino acid sequence of the parental AAV capsid protein.

The term “AAV genome” as used herein can refer to nucleic acid polynucleotide encoding genetic information related to the virus. The genome, in some instances, comprises a nucleic acid sequence flanked by AAV ITR sequences. The AAV genome can be a recombinant AAV genome generated using recombinatorial genetics methods, and which can include a heterologous nucleic acid (e.g., transgene) that comprises and/or is flanked by the ITR sequences.

The term “rAAV” refers to a “recombinant AAV”. In some embodiments, a recombinant AAV has an AAV genome in which part or all of the rep and cap genes have been replaced with heterologous sequences. The term “AAV particle”, “AAV nanoparticle”, or an “AAV vector” as used interchangeably herein refers to an AAV virus or virion comprising an AAV capsid within which is packaged a heterologous DNA polynucleotide, or “genome”, comprising nucleic acid sequence flanked by AAV inverted terminal repeat (ITR) sequences. In some cases, the AAV particle is modified relative to a parental AAV particle.

The term “cap gene” refers to the nucleic acid sequences that encode capsid proteins that form, or contribute to the formation of, the capsid, or protein shell, of the virus. In the case of AAV, the capsid protein may be VP1, VP2, or VP3. For other parvoviruses, the names and numbers of the capsid proteins can differ.

The term “rep gene” refers to the nucleic acid sequences that encode the non-structural proteins (rep78, rep68, rep52 and rep40) required for the replication and production of virus.

As used herein, “native” or “wild type” can be used interchangeably, and can refer to the form of a polynucleotide, gene or polypeptide as found in nature with its own regulatory sequences, if present.

As used herein, “endogenous” refers to the native form of a polynucleotide, gene or polypeptide in its natural location in the organism or in the genome of an organism. “Endogenous polynucleotide” includes a native polynucleotide in its natural location in the genome of an organism. “Endogenous gene” includes a native gene in its natural location in the genome of an organism. “Endogenous polypeptide” includes a native polypeptide in its natural location in the organism.

As used herein, “heterologous” refers to a polynucleotide, gene or polypeptide not normally found in the host organism but that is introduced into the host organism. “Heterologous polynucleotide” includes a native coding region, or portion thereof, that is reintroduced into the source organism in a form that is different from the corresponding native polynucleotide. “Heterologous gene” includes a native coding region, or portion thereof, that is reintroduced into the source organism in a form that is different from the corresponding native gene. For example, a heterologous gene may include a native coding region that is a portion of a chimeric gene including non-native regulatory regions that is reintroduced into the native host. “Heterologous polypeptide” includes a native polypeptide that is reintroduced into the source organism in a form that is different from the corresponding native polypeptide. The subject genes and proteins can be fused to other genes and proteins to produce chimeric or fusion proteins. The genes and proteins useful in accordance with embodiments of the subject disclosure include not only the specifically exemplified full-length sequences, but also portions, segments and/or fragments (including contiguous fragments and internal and/or terminal deletions compared to the full-length molecules) of these sequences, variants, mutants, chimerics, and fusions thereof.

The term “exogenous” gene as used herein is meant to encompass all genes that do not naturally occur within the genome of an individual. For example, a miRNA could be introduced exogenously by a virus, e.g. an AAV nanoparticle.

As used herein, a “subject” refers to an animal that is the object of treatment, observation or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human. In some embodiments, the subject is a rodent (e.g., rat or mouse). In some embodiments, the subject is a primate (e.g., human or money).

As used herein, the term “treatment” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

As used herein, the term “effective amount” refers to an amount sufficient to effect beneficial or desirable biological and/or clinical results.

As used herein, the term “pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™. Auxiliary, stabilizer, emulsifier, lubricant, binder, pH adjustor controller, isotonic agent and other conventional additives may also be added to the carriers.

### Acoustic Targeting Peptides and Capsid Proteins

It was reasoned that the limitations of the currently available FUS-BBBO compositions and methods described above arise from the fact that wild-type serotypes of AAV did not evolve to cross physically loosened biophysical barriers and are therefore not optimal for this purpose. It was hypothesized that these limitations could be addressed by developing new engineered viral serotypes specifically optimized for FUS-BBBO delivery. Capsid engineering techniques in which mutations are introduced into viral capsid proteins have been used to enhance gene delivery properties such as tissue specificity, immune evasion, or axonal tracing. However, they have not yet been used to optimize viral vectors to work in conjunction with specific physical delivery mechanisms. As described herein, high throughput in vivo selection was used to engineer new AAV vectors specifically designed for local neuronal transduction at the site of FUS-BBBO (Example 1). The resulting vectors substantially enhance ultrasound-targeted gene delivery and neuronal tropism while reducing peripheral transduction, providing a more than ten-fold improvement in targeting specificity. In addition to enhancing the only known approach to noninvasively target gene delivery to specific brain regions, the results shown in Example 1 establish the ability of AAV vectors to be evolved for specific physical delivery mechanisms.

Disclosed herein include adeno-associated virus (AAV) acoustic targeting peptides. In some embodiments, the AAV acoustic targeting peptide comprises an amino acid sequence that comprises at least 4 contiguous amino acids from a sequence selected from the group consisting of AGNTSDR (SEQ ID NO: 1; AAV.FUS.1), ATDAYNK (SEQ ID NO: 2; AAV.FUS.2), WSEGGQP (SEQ ID NO: 3; AAV.FUS.3), SVGSADP (SEQ ID NO: 4; AAV.FUS.4), and VRMEGEV (SEQ ID NO: 5; AAV.FUS.5). The acoustic targeting AAV peptide can be part of an AAV, for example part of a capsid protein of the AAV. In some embodiments, the capsid protein is the VP1 capsid protein. The acoustic targeting peptide can be conjugated to a nanoparticle, a second molecule, a viral capsid protein, or a combination thereof. The AAV capsid protein can comprise a substitution, for example an substitution of 7, 6, 5, 4, 3, or 2 contiguous amino acids.

The AAV acoustic targeting peptide can comprise at least 4 contiguous amino acids from the sequence of AGNTSDR (SEQ ID NO: 1). The AAV acoustic targeting peptide can comprise at least 5 contiguous amino acids from the sequence of AGNTSDR (SEQ ID NO: 1). The AAV acoustic targeting peptide can comprise at least 6 contiguous amino acids from the sequence of AGNTSDR (SEQ ID NO: 1). The AAV acoustic targeting peptide can comprise AGNTSDR (SEQ ID NO: 1).

The AAV acoustic targeting peptide can comprise at least 4 contiguous amino acids from the sequence ATDAYNK (SEQ ID NO: 2). The AAV acoustic targeting peptide can comprise at least 5 contiguous amino acids from the sequence of ATDAYNK (SEQ ID NO: 2). The AAV acoustic targeting peptide can comprise at least 6 contiguous amino acids from the sequence of ATDAYNK (SEQ ID NO: 2). The AAV acoustic targeting peptide can comprise ATDAYNK (SEQ ID NO: 2).

The AAV acoustic targeting peptide can comprise at least 4 contiguous amino acids from the sequence WSEGGQP (SEQ ID NO: 3). The AAV acoustic targeting peptide can comprise at least 5 contiguous amino acids from the sequence of WSEGGQP (SEQ ID NO: 3). The AAV acoustic targeting peptide can comprise at least 6 contiguous amino acids from the sequence of WSEGGQP (SEQ ID NO: 3). The AAV acoustic targeting peptide can comprise WSEGGQP (SEQ ID NO: 3).

The AAV acoustic targeting peptide can comprise at least 4 contiguous amino acids from the sequence SVGSADP (SEQ ID NO: 4). The AAV acoustic targeting peptide can comprise at least 5 contiguous amino acids from the sequence of SVGSADP (SEQ ID NO: 4). The AAV acoustic targeting peptide can comprise at least 6 contiguous amino acids from the sequence of SVGSADP (SEQ ID NO: 4). The AAV acoustic targeting peptide can comprise SVGSADP (SEQ ID NO: 4).

The AAV acoustic targeting peptide can comprise at least 4 contiguous amino acids from the sequence VRMEGEV (SEQ ID NO: 5). The AAV acoustic targeting peptide can comprise at least 5 contiguous amino acids from the sequence of VRMEGEV (SEQ ID NO: 5). The AAV acoustic targeting peptide can comprise at least 6 contiguous amino acids from the sequence of VRMEGEV (SEQ ID NO: 5). The AAV acoustic targeting peptide can comprise VRMEGEV (SEQ ID NO: 5).

The AAV acoustic targeting peptide can be part of an AAV. The AAV acoustic targeting peptide can be part of a capsid protein of the AAV. The AAV acoustic targeting peptide can be conjugated to a nanoparticle, a second molecule, a viral capsid protein, or a combination thereof. The AAV acoustic targeting peptide can be configured enhance the focused ultrasound (FUS)-target specificity of FUS blood-brain-barrier opening (FUS-BBBO)-mediated transduction.

In some embodiments, an AAV comprising the AAV acoustic targeting peptide demonstrates, as compared to the corresponding parental AAV that does not comprise the acoustic targeting peptide, an at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) (i) increase transduction at site(s) of FUS-BBBO; (ii) increase in neuronal tropism; and/or (iii) decrease in transduction in peripheral organs.

Disclosed herein include adeno-associated virus (AAV) capsid proteins. In some embodiments, the AAV capsid protein comprises an AAV acoustic targeting peptide disclosed herein. Disclosed herein include nucleic acids. In some embodiments, the nucleic acid comprises a sequence encoding an AAV acoustic targeting peptide disclosed herein. In some embodiments, the nucleic acid comprises a sequence encoding an AAV capsid protein disclosed herein.

The AAV serotype used to derive the AAV capsid protein can vary. The AAV capsid can be derived from AAV9, or a variant thereof. The AAV capsid can be derived from an AAV selected from AAV1, AAV2, AAV3, AAV3b, AAV4, AAV5, AAV6, AAV7, AAV8, AAV9, AAV10, human isolate hu.31, human isolate hu.32, rhesus isolate rh.8, and rhesus isolate rh.10. In some embodiments, the AAV capsid protein can be derived from an AAV serotype selected from AAV9, AAV9 K449R (or K449R AAV9), AAV1, AAVrhlO, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B 3 (PHP.B 3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2 Al 5/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9.16, AAV9.24, AAV9.45, AAV9.47, AAV9.61, AAV9.68, AAV9.84, AAV9.9, AAV10, AAV11, AAV 12, AAV16.3, AAV24.1, AAV27.3, AAV42.12, AAV42-lb, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAVl-7/rh.48, AAVl-8/rh.49, AAV2-15/rh.62, AAV2-3/rh.61, AAV2-4/rh.50, AAV2-5/rh.51, AAV3. l/hu.6, AAV3. l/hu.9, AAV3-9/rh.52, AAV3-1 l/rh.53, AAV4-8/rl 1.64, AAV4-9/rh. 54, AAV4-l9/rh. 55, AAV5-3/rh.57, AAV5-22/rh. 58, AAV7.3/hu.7, AAVl6.8/hu.lO, AAVl6.l2/hul 1, AAV29.3/bb.l, AAV29.5/bb.2, AAVl06. l/hu.37, AAV1 l4.3/hu.40, AAVl27.2/hu.4l, AAVl27.5/hu.42, AAVl28.3/hu.44, AAVl30.4/hu.48, AAVl45. l/hu.53, AAVl45.5/hu.54, AAVl45.6/hu.55, AAVl6l. l0/hu.60, AAVl6l.6/hu.6l, AAV33. l2/hu.l7, AAV33.4/hu.l5, AAV33.8/hu.l6, AAV52/hu.l9, AAV52.l/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi. l, AAVpi.3, AAVpi.2, AAVrh.60, AAVrh.44, AAVrh.65, AAVrh.55, AAVrh.47, AAVrh.69, AAVrh.45, AAVrh.59, AAVhu. l2, AAVH6, AAVH-l/hu.l, AAVH-5/hu.3, AAVLG-l0/rh.40, AAVLG-4/rh.38, AAVLG-9/hu.39, AAVN72l-8/rh.43, AAVCh.5, AAVCh.5Rl, AAVcy.2, AAVcy.3, AAVcy.4, AAVcy.5, AAVCy.5Rl, AAVCy.5R2, AAVCy.5R3, AAVCy.5R4, AAVcy.6, AAVhu. l, AAVhu.2, AAVhu.3, AAVhu.4, AAVhu.5, AAVhu.6, AAVhu.7, AAVhu.9, AAVhu.10, AAVhu. l l, AAVhu. l3, AAVhu.l5, AAVhu.l6, AAVhu. l 7, AAVhu.l 8, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.6l, AAVhu.63, AAVhu.64, AAVhu.66, AAVhu.67, AAVhu. l 4/9, AAVhu.t 19, AAVrh.2, AAVrh.2R, AAVrh.8, AAVrh.8R, AAVrh. lO, AAVrh.l2, AAVrh. l3, AAVrh.l3R, AAVrh. l4, AAVrh.l7, AAVrh. l8, AAVrh.l9, AAVrh.20, AAVrh.2l, AAVrh.22, AAVrh.23, AAVrh.24, AAVrh.25, AAVrh.3 l, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.l.2, AAVrh.48.2, AAVrh.49, AAVrh.5l, AAVrh.52, AAVrh.53, AAVrh.54, AAVrh.56, AAVrh.57, AAVrh.58, AAVrh.6l, AAVrh.64, AAVrh.64Rl, AAVrh.64R2, AAVrh.67, AAVrh.73, AAVrh.74, AAVrh8R, AAVrh8R A586R mutant, AAVrh8R R533 A mutant, AAAV, BAAV, caprine AAV, bovine AAV, AAVhEl.l, AAVhErl.5, AAVhERl. l4, AAVhErl.8, AAVhErl.l6, AAVhErl.l8, AAVhErl.35, AAVhErl.7, AAVhErl.36, AAVhEr2.29, AAVhEr2.4, AAVhEr2.l6, AAVhEr2.30, AAVhEr2.3 l, AAVhEr2.36, AAVhERl.23, AAVhEr3.l, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.l9, AAVhu. l l, AAVhu.53, AAV4-8/rh.64, AAVLG-9/hu.39, AAV54.5/hu.23, AAV54.2/hu.22, AAV54.7/hu.24, AAV54.1/hu.2l, AAV54.4R/hu.27, AAV46.2/hu.28, AAV46.6/hu.29, AAVl28. l/hu.43, true type AAV (ttAAV), EGRENN AAV 10, Japanese AAV 10 serotypes, AAV CBr-7.l, AAV CBr-7. 10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-El, AAV CBr-E2, AAV CBr-E3, AAV CBr-E4, AAV CBr-E5, AAV CBr-e5, AAV CBr-E6, AAV CBr-E7, AAV CBr-E8, AAV CHt-l, AAV CHt-2, AAV CHt-3, AAV CHt-6. l, AAV CHt-6.lO, AAV CHt-6.5, AAV CHt-6.6, AAV CHt-6.7, AAV CHt-6.8, AAV CHt-Pl, AAV CHt-P2, AAV CHt-P5, AAV CHt-P6, AAV CHt-P8, AAV CHt-P9, AAV CKd-l, AAV CKd-10, AAV CKd-2, AAV CKd-3, AAV CKd-4, AAV CKd-6, AAV CKd-7, AAV CKd-8, AAV CKd-Bl, AAV CKd-B2, AAV CKd-B3, AAV CKd-B4, AAV CKd-B5, AAV CKd-B6, AAV CKd-B7, AAV CKd-B8, AAV CKd-Hl, AAV CKd-H2, AAV CKd-H3, AAV CKd-H4, AAV CKd-H5, AAV CKd-H6, AAV CKd-N3, AAV CKd-N4, AAV CKd-N9, AAV CLg-Fl, AAV CLg-F2, AAV CLg-F3, AAV CLg-F4, AAV CLg-F5, AAV CLg-F6, AAV CLg-F7, AAV CLg-F8, AAV CLv-l, AAV CLvl-l, AAV Clvl-lO, AAV CLvl-2, AAV CLv-l2, AAV CLvl-3, AAV CLv-l 3, AAV CLvl-4, AAV Clvl-7, AAV Clvl-8, AAV Clvl-9, AAV CLv-2, AAV CLv-3, AAV CLv-4, AAV CLv-6, AAV CLv-8, AAV CLv-Dl, AAV CLv-D2, AAV CLv-D3, AAV CLv-D4, AAV CLv-D5, AAV CLv-D6, AAV CLv-D7, AAV CLv-D8, AAV CLy-El, AAV CLv-K1, AAV CLv-K3, AAV CLv-K6, AAV CLv-L4, AAV CLv-L5, AAV CLv-L6, AAV CLv-M1, AAV CLv-Ml l, AAV CLv-M2, AAV CLv-M5, AAV CLv-M6, AAV CLv-M7, AAV CLv-M8, AAV CLv-M9, AAV CLv-Rl, AAV CLv-R2, AAV CLv-R3, AAV CLv-R4, AAV CLv-R5, AAV CLv-R6, AAV CLv-R7, AAV CLv-R8, AAV CLv-R9, AAV CSp-l, AAV CSp-lO, AAV CSp-l l, AAV CSp-2, AAV CSp-3, AAV CSp-4, AAV CSp-6, AAV CSp-7, AAV CSp-8, AAV CSp-8. l0, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, variants thereof, a hybrid or chimera of any of the foregoing AAV serotypes, or any combination thereof. The rAAV disclosed herein can have a capsid from a different serotype of AAV than the rAAV genome.

The engineered AAV capsid proteins described herein have, in some cases, an insertion or substitution of an amino acid that is heterologous to the wild-type AAV capsid protein at the amino acid position of the insertion or substitution. In some embodiments, the amino acid is not endogenous to the wild-type AAV capsid protein at the amino acid position of the insertion or substitution. The amino acid can be a naturally occurring amino acid in the same or equivalent amino acid position as the insertion of the substitution in a different AAV capsid protein.

The rAAV can comprise a chimeric AAV capsid. A “chimeric” AAV capsid refers to a capsid that has an exogenous amino acid or amino acid sequence. The rAAV may comprise a mosaic AAV capsid. A “mosaic” AAV capsid refers to a capsid that made up of two or more capsid proteins or polypeptides, each derived from a different AAV serotype. The rAAV may be a result of transcapsidation, which, in some cases, refers to the packaging of an inverted terminal repeat (ITR) from a first serotype into a capsid of a second serotype, wherein the first and second serotypes are not the same. In some cases, the capsid genes of the parental AAV serotype is pseudotyped, which means that the ITRs from a first AAV serotype (e.g., AAV1) are used in a capsid from a second AAV serotype (e.g., AAV9), wherein the first and second AAV serotypes are not the same. As a non-limiting example, a pseudotyped AAV serotype comprising the AAV1ITRs and AAV9 capsid protein may be indicated AAV1/9. The rAAV may additionally, or alternatively, comprise a capsid that has been engineered to express an exogenous ligand binding moiety (e.g., receptor), or a native receptor that is modified.

In some embodiments, the rAAV capsid proteins comprises a substitution or insertion of one or more amino acids in an amino acid sequence of an AAV capsid protein. The AAV capsid protein from which the engineered AAV capsid protein of the present disclosure is produced can be referred to as a “parental” or “wild-type” AAV capsid protein, or a “corresponding unmodified capsid protein.” In some cases, the parental AAV capsid protein has a serotype selected from the group consisting of AAV1, AAV2, rAAV3, AAV4, AAV5, AAV6, AAV7, AAV8, AAV9, AAV10, AAV11, and AAV12. The complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562. At least portions of the AAV-DJ genome are provided in Grimm, D. et al. J. Virol. 82, 5887-5911 (2008).

### Adeno-Associated Virus (AAV) Vectors and Recombinant AAVs

AAV is a replication-deficient parvovirus, the single-stranded DNA genome of which is about 4.7 kb in length including 145 nucleotide ITRs. The ITRs play a role in integration of the AAV DNA into the host cell genome. When AAV infects a host cell, the viral genome integrates into the host's chromosome resulting in latent infection of the cell. In a natural system, a helper virus (e.g., adenovirus or herpesvirus) provides genes that allow for production of AAV virus in the infected cell. In the case of adenovirus, genes E1A, E1B, E2A, E4 and VA provide helper functions. Upon infection with a helper virus, the AAV provirus is rescued and amplified, and both AAV and adenovirus are produced. In the instances of recombinant AAV vectors having no Rep and/or Cap genes, the AAV can be non-integrating. Disclosed herein include recombinant AAV (rAAV). In some embodiments, the rAAV comprises an AAV capsid protein described herein.

In some embodiments, the AAV vector comprises coding regions of one or more proteins of interest. The AAV vector can include a 5′ ITR of AAV, a 3′ AAV ITR, a promoter, and a restriction site downstream of the promoter to allow insertion of a polynucleotide encoding one or more proteins of interest, wherein the promoter and the restriction site are located downstream of the 5′ AAV ITR and upstream of the 3′ AAV ITR. In some embodiments, the AAV vector includes a posttranscriptional regulator-element downstream of the restriction site and upstream of the 3′ AAV ITR. In some embodiments, the AAV vectors disclosed herein can be used as AAV transfer vectors carrying a transgene encoding a protein of interest for producing recombinant AAV viruses that can express the protein of interest in a host cell.

Generation of the viral vector can be accomplished using any suitable genetic engineering techniques well known in the art, including, without limitation, the standard techniques of restriction endonuclease digestion, ligation, transformation, plasmid purification, and DNA sequencing, for example as described in Sambrook el al. (Molecular Cloning: A Laboratory Manual. Cold Spring Harbor Laboratory Press, N.Y. (1989)). The viral vector can incorporate sequences from the genome of any known organism. The sequences can be incorporated in their native form or can be modified in any way to obtain a desired activity. For example, the sequences can comprise insertions, deletions or substitutions.

The viral vectors can include additional sequences that make the vectors suitable for replication and integration in eukaryotes. In other embodiments, the viral vectors disclosed herein can include a shuttle element that makes the vectors suitable for replication and integration in both prokaryotes and eukaryotes. In some embodiments, the viral vectors can include additional transcription and translation initiation sequences, such as promoters and enhancers; and additional transcription and translation terminators, such as polyadenylation signals. Various regulatory elements that can be included in an AAV vector have been described in US2012/0232133 which is hereby incorporated by reference in its entirety.

Vectors comprising a nucleic acid sequence encoding the modified AAV capsid proteins of the present disclosure are also provided herein. For example, the vectors of the present disclosure can comprise a nucleic acid sequence encoding the two AAV viral genes, Rep (Replication), and a Cap (Capsid) gene, wherein the Cap gene, encoding viral capsid proteins VP1, VP2, and VP3 is modified to produce the modified AAV capsid proteins of the present disclosure.

Disclosed herein are methods of producing a rAAV. In some embodiments, all elements that are required for AAV production in target cell (e.g., HEK293 cells) are transiently transfected into the target cell using suitable methods known in the art. For example, the rAAV of the present disclosure can be produced by co-transfecting three plasmid vectors, a first vector with ITR-containing plasmid carrying the transgene (e.g., a DNA sequence that encodes a trophic factor, a growth factor, or other soluble factors), a second vector that carries the AAV Rep and Cap genes (e.g., one or more variant capsid proteins provided herein); and (3), a third vector that provides the helper genes isolated from adenovirus. In some cases, rAAVs of the present disclosure are generated using the methods described in Challis et al. Systemic AAV vectors for widespread and targeted gene delivery in rodents. Nat. Protoc. 14, 379 (2019), which is hereby incorporated by reference in its entirety. Briefly, triple transfection of HEK293T cells using polyethylenimine (PEI) is performed, viruses are collected after 120 hours from both cell lysates and media and purified over iodixanol.

Disclosed herein, are methods of manufacturing comprising: (a) introducing into a cell a nucleic acid comprising: (i) a first nucleic acid sequence (heterologous nucleic acid) encoding, e.g., a protein, enclosed by a 5′ and a 3′ inverted terminal repeat (ITR) sequence; (ii) a second nucleic acid sequence encoding a viral genome comprising a 5′ ITR sequence, a Replication (Rep) gene, one or more (Cap) genes, and a 3′ ITR, wherein the one or more Cap genes encodes a variant AAV capsid protein described herein; and (iii) a third nucleic acid sequence encoding a first helper virus protein selected from the group consisting of E4orf6, E2a, and VA RNA, and optionally, a second helper virus protein comprising Ela or E1b55k; (b) expressing in the cell the AAV capsid protein described herein; (c) assembling an AAV particle comprising the AAV capsid proteins disclosed herein; and (d) packaging the first nucleic acid sequence in the AAV particle. In some instances, the methods further comprise packing the first nucleic acid sequence encoding the therapeutic gene expression product such that it becomes encapsidated by the rAAV capsid protein. In some embodiments, the rAAV particles are isolated, concentrated, and purified using suitable viral purification methods, such as those described herein.

In some embodiments, the rAAVs are generated by triple transfection of precursor cells (e.g., HEK293T) cells using a standard transfection protocol (e.g., with PEI). Viral particles are harvested from the media after a period of time (e.g., 72 h post transfection) and from the cells and media at a later point in time (e.g., 120 h post transfection). Virus present in the media is concentrated by precipitation with 8% poly(ethylene glycol) and 500 mM sodium chloride and the precipitated virus is added to the lysates prepared from the collected cells. The viruses are purified over iodixanol (Optiprep, Sigma) step gradients (15%, 25%, 40% and 60%). Viruses are concentrated and formulated in PBS. Virus titers are determined by measuring the number of DNaseI-resistant vector genome copies (VGs) using qPCR and the linearized genome plasmid as a control.

The Rep protein can be selected from Rep78, Rep68, Rep52, and Rep40. The genome of the AAV helper virus comprises an AAV helper gene selected from E2, E4, and VA. In some instances, the first nucleic acid sequence and the second nucleic acid sequence are in trans. In some instances, the first nucleic acid sequence and the second nucleic acid sequence are in cis. In some instances, the first nucleic acid sequence, the second nucleic acid sequence and the third nucleic acid sequence, are in trans.

The cell can be a cell from a human, a primate, a murine, a feline, a canine, a porcine, an ovine, a bovine, an equine, a caprine and a lupine host cell. The cell can be a progenitor or precursor cell, such as a stem cell. In some instances, the stem cell is a mesenchymal cell, embryonic stem cell, induced pluripotent stem cell (iPSC), fibroblast or other tissue specific stem cell. The cell can be immortalized. In some instances, the embryonic stem cell is a human embryonic stem cell. In some instances, the human embryonic stem cell is a human embryonic kidney 293 (HEK-293) cell. In some instances, the cell is a differentiated cell. Base on the disclosure provided, it is expected that this system can be used in conjunction with any transgenic line expressing a recombinase in the target cell type of interest to develop AAV capsids that more efficiently transduce that target cell population.

There are provided nucleic acids comprising a sequence encoding any of the AAV capsid proteins of the disclosure (e.g., comprising an acoustic targeting peptide). For example, there are provided herein plasmid vectors encoding the variant capsid proteins of the present disclosure (e.g., comprising acoustic targeting peptides). Also disclosed are nucleic acids encoding the rAAV capsids comprising variant AAV capsid proteins (e.g., comprising acoustic targeting peptides) of the present disclosure. Heterologous nucleic acids and transgenes of the present embodiment may also include plasmid vectors. Plasmid vectors are useful for the generation of the rAAV particles described herein. An AAV vector can comprise a genome of a helper virus. Helper virus proteins are required for the assembly of a recombinant rAAV, and packaging of a transgene containing a heterologous nucleic acid into the rAAV. The helper virus genes are adenovirus genes E4, E2a and VA, that when expressed in the cell, assist with AAV replication. In some embodiments, an AAV vector comprises E2. In some embodiments, an AAV vector comprises E4. In some embodiments, an AAV vector comprises VA. In some instances, the AAV vector comprises one of helper virus proteins, or any combination thereof. In some instances, the plasmid vector is bacterial. In some instances, the plasmid vector is derived from Escherichia coli. In some instances, the nucleic acid sequence comprises, in a 5′ to 3′ direction: (1) a 5′ ITR sequence, (2) a Replication (Rep) gene, (3) a Capsid (Cap) gene, and (4) a 3′ ITR, wherein the Cap gene encodes the variant AAV capsid protein described herein. In some instances, the plasmid vector encodes a pseudotyped AAV capsid protein.

Disclosed herein are modified viral genomes comprising genetic information (e.g., heterologous nucleic acid) that are assembled into a rAAV via viral packaging. In some instances, the viral genome is from an AAV serotype selected from AAV1, AAV2, AAV3, AAV4, AAV5, AAV6, AAV7, AAV8, AAV9, AAV10, AAV11, and AAV12.

A viral genome, such as those described herein, can comprise a transgene, which in some cases encodes a heterologous gene expression product (e.g., therapeutic gene expression product, recombinant capsid protein, and the like). The transgene is in cis with two ITRs flanking the transgene. The transgene may comprise a therapeutic nucleic acid encoding a therapeutic gene expression product

The viral genome, in some cases, is a single stranded viral DNA comprising the transgene. The AAV vector can be episomal. In some instances, the viral genome is a concatemer. An episomal viral genome can develop chromatin-like organization in the target cell that does not integrate into the genome of the target cell. When infected into non-dividing cells, the stability of the episomal viral genome in the target cell enable the long-term transgene expression. Alternatively, the AAV vector integrates the transgene into the genome of the target cell predominantly at a specific site (e.g., AAVS 1 on human chromosome 19).

The rAAV genome can, for example, comprise at least one inverted terminal repeat configured to allow packaging into a vector and a cap gene. In some embodiments, it can further include a sequence within a rep gene required for expression and splicing of the cap gene. In some embodiments, the genome can further include a sequence capable of expressing a capsid protein provided herein.

The rAAV capsid proteins can be isolated and purified. The AAV can be isolated and purified by methods standard in the art such as by column chromatography or cesium chloride gradients. Methods for purifying AAV from helper virus are known in the art and may include methods disclosed in, for example, Clark et al., Hum. Gene Ther., 10(6): 1031-1039 (1999); Schenpp and Clark, Methods Mol. Med., 69: 427-443 (2002); U.S. Pat. No. 6,566,118 and WO 98/09657.

The rAAV capsid and/or rAAV capsid protein can be conjugated to a nanoparticle, a second molecule, or a viral capsid protein. In some cases, the nanoparticle or viral capsid protein would encapsidate the therapeutic nucleic acid described herein. In some instances, the second molecule is a therapeutic agent, e.g., a small molecule, antibody, antigen-binding fragment, peptide, or protein, such as those described herein. In some instances, the second molecule is a detectable moiety. For example, the modified AAV capsid and/or rAAV capsid protein conjugated to a detectable moiety may be used for in vitro, ex vivo, or in vivo biomedical research applications, the detectable moiety used to visualize the modified capsid protein. The modified AAV capsid and/or rAAV capsid protein conjugated to a detectable moiety may also be used for diagnostic purposes.

One or more insertions, substitutions, or point mutations can be employed in a single system (e.g., in a single AAV vector, a single AAV capsid protein, or a single rAAV). For example one can employ one or more acoustic targeting sequences and also modify other sites to reduce the recognition of the AAVs by the pre-existing antibodies present in a subject, such as a human. The AAV vector can include a capsid, which influences the tropism/acoustic targeting, speed of expression and possible immune response. The vector can also include the rAAV, which genome carries the transgene/therapeutic aspects (e.g., sequences) along with regulatory sequences. The vector can include the acoustic targeting sequence within/on a substrate that is or transports the desired molecule (e.g., therapeutic molecule, diagnostic molecule).

In some embodiments, the rAAV comprises an AAV capsid protein comprising any of the AAV acoustic targeting peptide described herein. In some embodiments, the rAAVs exhibit tropism for the target brain region(s).

Disclosed herein include recombinant adeno-associated virus (rAAV). In some embodiments, the rAAV comprises an AAV acoustic targeting peptide disclosed herein, or an AAV capsid protein disclosed herein. In some embodiments, the rAAV comprises an AAV capsid protein which comprises an AAV acoustic targeting peptide disclosed herein, wherein the amino acid sequence is inserted between two adjacent amino acids in AA586-592, or functional equivalents thereof, of the AAV capsid protein. The two adjacent amino acids can be AA588 and AA589. In some embodiments, the AAV capsid protein comprises, or consists thereof, SEQ ID NO: 6 (AAV.FUS.1), SEQ ID NO: 7 (AAV.FUS.2), SEQ ID NO: 8 (AAV.FUS.3), SEQ ID NO: 9 (AAV.FUS.4), and/or SEQ ID NO: 10 (AAV.FUS.5). The rAAV can comprise an rAAV vector genome.

The location of the acoustic targeting peptide within the capsid protein can vary. In some embodiments, the amino acid sequence is inserted between two adjacent amino acids in AA586-592 (e.g., between AA586 and AA587, AA587 and AA588, AA588 and AA589, AA589 and AA590, AA590 and AA591, AA591 and AA592) or functional equivalents thereof, of the AAV capsid protein. The two adjacent amino acids can be AA588 and AA589.

In some embodiments, AAV genomes contain both the full rep and cap sequence that have been modified so as to not prevent the replication of the virus under conditions in which it could normally replicate (co-infection of a mammalian cell along with a helper virus such as adenovirus). A pseudo wild-type (“wt”) genome can be one that has an engineered cap gene within a “wt” AAV genome. In some embodiments, the “pseudo-wild type” AAV genome contains the viral replication gene (rep) and capsid gene (cap) flanked by ITRs. In some embodiments, the rAAV genome contains the cap gene and only those sequences within the rep gene required for the expression and splicing of the cap gene products. In some embodiments, a capsid gene recombinase recognition sequence is provided with inverted terminal repeats flanking these sequences.

Uses of AAV Vectors and rAAVs

Disclosed herein include compositions. In some embodiments, the composition comprises: an AAV acoustic targeting peptide disclosed herein, an AAV capsid protein disclosed herein, a nucleic acid disclosed herein, an rAAV disclosed herein, or a combination thereof. The composition can be a pharmaceutical composition comprising one or more pharmaceutical acceptable carriers.

Disclosed herein include compositions for use in the delivery of an agent to a target environment of a subject. In some embodiments, the composition comprises: an AAV comprising (1) an AAV capsid protein disclosed herein and (2) an agent to be delivered to the target environment of the subject.

The target environment can comprise one or more target brain region(s). The composition can be a pharmaceutical composition comprising one or more pharmaceutical acceptable carriers. The agent to be delivered can comprise a nucleic acid, a peptide, a small molecule, an aptamer, or a combination thereof. In some embodiments, the nucleic acid encodes one or more payload proteins and/or one or more payload RNA agents (e.g., one or more components of a synthetic protein circuit). The payload protein and/or the payload RNA agent can be capable of diminishing the concentration, stability, and/or activity an endogenous protein. The payload protein can be a therapeutic protein or a variant thereof. The therapeutic protein can be configured to prevent or treat a disease or disorder of a subject, optionally the subject suffers from a deficiency of said therapeutic protein. The payload RNA agent can comprise one or more of dsRNA, siRNA, shRNA, pre-miRNA, pri-miRNA, miRNA, stRNA, lncRNA, piRNA, and snoRNA. A payload protein can comprise a chemogenetic protein. The chemogenetic protein can be selected from the comprising DREADD, PSAM, TrpV1, hM2Di, hM4Di, hM1Dq, hM3Dq, hM5Dq, or any combination thereof. The nucleic acid can comprise a tandem gene expression element selected from the group an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof. The nucleic acid can comprise a promoter operably linked to a polynucleotide encoding the one or more payload protein and/or the one or more payload RNA agents. The promoter can be capable of inducing the transcription of the polynucleotide.

Disclosed herein include compositions comprising an AAV acoustic targeting peptide, an AAV capsid protein, a nucleic acid, an rAAV, as described herein, or a combination thereof. Disclosed herein include compositions for use in the delivery of an agent to a target environment of a subject in need. In some embodiments, the composition comprises an AAV comprising (1) an AAV capsid protein disclosed herein and (2) an agent to be delivered to the target environment of the subject. The compositions can be pharmaceutical compositions comprising one or more pharmaceutical acceptable carriers.

The target environment can be target brain region(s). The target environment can be brain endothelial cells, neurons, capillaries in the brain, arterioles in the brain, arteries in the brain, or a combination thereof.

The agent to be delivered can comprise a nucleic acid, a peptide, a small molecule, an aptamer, or a combination thereof. The AAV vectors disclosed herein can be effectively transduced to a target environment (e.g., target brain region(s)), for example, for delivering nucleic acids. In some embodiments, a method of delivering a nucleic acid sequence to the target brain region(s) is provided. The protein can be part of a capsid of an AAV. The AAV can comprise a nucleic acid sequence to be delivered target brain region(s). One can then administer the AAV to the subject.

In some embodiments, the nucleic acid sequence to be delivered to a target environment (e.g., target brain region(s)) comprises one or more sequences that would be of some use or benefit to the target brain region(s) and/or the local of delivery or surrounding tissue or environment. In some embodiments, it can be a nucleic acid that encodes a protein of interest. The nucleic acid can comprise one or more of: a) a DNA sequence that encodes a trophic factor, a growth factor, or a soluble protein; b) a cDNA that restores protein function to humans or animals harboring a genetic mutation(s) in that gene; c) a cDNA that encodes a protein that can be used to control or alter the activity or state of a cell; d) a cDNA that encodes a protein or a nucleic acid that can be used for assessing the state of a cell; e) a cDNA that encodes a protein for gene editing, or a guide RNA; f) a DNA sequence for genome editing via homologous recombination; g) a DNA sequence encoding a therapeutic RNA; h) an shRNA or an artificial miRNA delivery system; and i) a DNA sequence that influences the splicing of an endogenous gene.

In some embodiments, the vector can also comprise regulatory control elements known to one of skill in the art to influence the expression of the RNA and/or protein products encoded by the polynucleotide within desired cells of the subject.

In some embodiments, functionally, expression of the polynucleotide is at least in part controllable by the operably linked regulatory elements such that the element(s) modulates transcription of the polynucleotide, transport, processing and stability of the RNA encoded by the polynucleotide and, as appropriate, translation of the transcript. A specific example of an expression control element is a promoter, which is usually located 5′ of the transcribed sequence. Another example of an expression control element is an enhancer, which can be located 5′ or 3′ of the transcribed sequence, or within the transcribed sequence. Another example of a regulatory element is a recognition sequence for a microRNA. Another example of a regulatory element is an intron and the splice donor and splice acceptor sequences that regulate the splicing of said intron. Another example of a regulatory element is a transcription termination signal and/or a polyadenylation sequence.

Expression control elements and promoters include those active in a particular tissue or cell type, referred to herein as a “tissue-specific expression control elements/promoters.” Tissue-specific expression control elements are typically active in a specific cell or tissue (for example in the liver, brain, central nervous system, spinal cord, eye, retina or lung). Expression control elements are typically active in these cells, tissues or organs because they are recognized by transcriptional activator proteins, or other regulators of transcription, that are unique to a specific cell, tissue or organ type.

Expression control elements also include ubiquitous or promiscuous promoters/enhancers which are capable of driving expression of a polynucleotide in many different cell types. Such elements include, but are not limited to the cytomegalovirus (CMV) immediate early promoter/enhancer sequences, the Rous sarcoma virus (RSV) promoter/enhancer sequences, the CMV, chicken β-actin, rabbit β-globin (CAG) promoter/enhancer sequences, and the other viral promoters/enhancers active in a variety of mammalian cell types; promoter/enhancer sequences from ubiquitously or promiscuously expressed mammalian genes including, but not limited to, beta actin, ubiquitin or EF1 alpha; or synthetic elements that are not present in nature.

Expression control elements also can confer expression in a manner that is regulatable, that is, a signal or stimuli increases or decreases expression of the operably linked polynucleotide. A regulatable element that increases expression of the operably linked polynucleotide in response to a signal or stimuli is also referred to as an “inducible element” (that is, it is induced by a signal). Particular examples include, but are not limited to, a hormone (e.g., steroid) inducible promoter. A regulatable element that decreases expression of the operably linked polynucleotide in response to a signal or stimuli is referred to as a “repressible element” (that is, the signal decreases expression such that when the signal, is removed or absent, expression is increased). Typically, the amount of increase or decrease conferred by such elements is proportional to the amount of signal or stimuli present: the greater the amount of signal or stimuli, the greater the increase or decrease in expression.

The heterologous nucleic acid can comprise a 5′ ITR and a 3′ ITR. The agent can comprise a DNA sequence encoding a protein (e.g., a trophic factor, a growth factor, or a soluble protein). The heterologous nucleic acid can comprise a promoter operably linked to the polynucleotide encoding, e.g., a protein or an RNA agent. The promoter can be capable of inducing the transcription of the polynucleotide. Transcription of the polynucleotide can generate a transcript. The heterologous nucleic acid can comprise one or more of a 5′ UTR, 3′ UTR, a minipromoter, an enhancer, a splicing signal, a polyadenylation signal, a terminator, one or more silencer effector binding sequences, a protein degradation signal, and an internal ribosome-entry element (IRES). The silencer effector can comprise a microRNA (miRNA), a precursor microRNA (pre-miRNA), a small interfering RNA (siRNA), a short-hairpin RNA (shRNA), precursors thereof, derivatives thereof, or a combination thereof. The silencer effector can be capable of binding the one or more silencer effector binding sequences, thereby reducing the stability of the transcript and/or reducing the translation of the transcript. In some embodiments, the silencing effector comprises one or more miRNA binding sites. miRNA binding sites are operably linked regulatory elements that are typically located in the 3′UTR of the transcribed sequence. Binding of miRNAs to the target transcript (in complex with the RNA-Induced Silencing Complex, RISC) can reduce expression of the target transcript via translation inhibition and/or transcript degradation.

The polynucleotide further can comprise a transcript stabilization element. The transcript stabilization element can comprise woodchuck hepatitis post-translational regulatory element (WPRE), bovine growth hormone polyadenylation (bGH-polyA) signal sequence, human growth hormone polyadenylation (hGH-polyA) signal sequence, or any combination thereof. The nucleic acid can be or can encode an RNA agent. The RNA agent can comprise one or more of dsRNA, siRNA, shRNA, pre-miRNA, pri-miRNA, miRNA, stRNA, lncRNA, piRNA, and snoRNA. The RNA agent inhibits or suppresses the expression of a gene of interest in a cell. In some embodiments, the gene of interest can be selected from the group comprising SOD1, MAPT, APOE, HTT, C90RF72, TDP-43, APP, BACE, SNCA, ATXN1, ATXN2, ATXN3, ATXN7, SCN1A-SCNSA, and SCN8A-SCN11A. The heterologous nucleic acid further can comprise a polynucleotide encoding one or more secondary proteins, and the protein and the one or more secondary proteins can comprise a synthetic protein circuit. The heterologous nucleic acid can comprise a single-stranded AAV (ssAAV) vector or a self-complementary AAV (scAAV) vector.

The promoter can comprise a ubiquitous promoter, including but not limited to a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CBH promoter, or any combination thereof.

The promoter can be an inducible promoter, including but not limited to, a tetracycline responsive promoter, a TRE promoter, a Tre3G promoter, an ecdysone responsive promoter, a cumate responsive promoter, a glucocorticoid responsive promoter, and estrogen responsive promoter, a PPAR-γ promoter, an RU-486 responsive promoter, or a combination thereof.

The promoter can comprise a tissue-specific promoter and/or a lineage-specific promoter. The tissue specific promoter can be a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. The tissue specific promoter can be a neuron-specific promoter, for example a synapsin-1 (Syn) promoter, a CaMKIIa promoter, a calcium/calmodulin-dependent protein kinase II a promoter, a tubulin alpha I promoter, a neuron-specific enolase promoter, a platelet-derived growth factor beta chain promoter, TRPV1 promoter, a Nav1.7 promoter, a Nav1.8 promoter, a Nav1.9 promoter, or an Advillin promoter. The tissue specific promoter can be a muscle-specific promoter. In some embodiments, the muscle-specific promoter can comprise a MCK promoter.

The promoter can comprise an intronic sequence. The promoter can comprise a bidirectional promoter and/or an enhancer. In some embodiments, the enhancer can be a CMV enhancer. One or more cells of a subject can comprise an endogenous version of a nucleic acid sequence (e.g., a gene), and the promoter can comprise or can be derived from the promoter of the endogenous version. In some embodiments, one or more cells of a subject comprise an endogenous version of the nucleic acid sequence, and the sequence is not truncated relative to the endogenous version.

The promoter can vary in length, for example be less than 1 kb. In other embodiments, the promoter is greater than 1 kb. The promoter can have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 bp, or a number or a range between any two of these values, or more than 800 bp. The promoter may provide expression of the therapeutic gene expression product for a period of time in targeted tissues such as, but not limited to, the CNS. Expression of the therapeutic gene expression product can be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or a number or a range between any two of these values, or more than 65 years.

As used herein, a “protein of interest” can be any protein, including naturally-occurring and non-naturally occurring proteins. In some embodiments, a polynucleotide encoding one or more proteins of interest can be present in one of the AAV vectors disclosed herein, wherein the polynucleotide is operably linked with a promoter. In some instances, the promoter can drive the expression of the protein(s) of interest in a host cell (e.g., an endothelial cell). In some embodiments, the protein of interest is an anti-tau antibody, an anti-AB antibody, and/or ApoE isoform.

The protein can comprise aromatic L-amino acid decarboxylase (AADC), survival motor neuron 1 (SMN1), frataxin (FXN), Cystic Fibrosis Transmembrane Conductance Regulator (CFTR), Factor X (FIX), RPE65, Retinoid Isomerohydrolase (RPE65), Sarcoglycan Alpha (SGCA), and sarco/endoplasmic reticulum Ca2+-ATPase (SERCA2a), ApoE2, GBA1, GRN, ASP A, CLN2, GLB1, SGSH, NAGLU, IDS, NPC1, GAN, CFTR, GDE, OTOF, DYSF, MYO7A, ABCA4, F8, CEP290, CDH23, DMD, ALMS1, or any combination thereof.

The protein can comprise a disease-associated protein. In some embodiments, the level of expression of the disease-associated protein correlates with the occurrence and/or progression of the disease. The protein can comprise methyl CpG binding protein 2 (MeCP2), DRK1A, KAT6A, NIPBL, HDAC4, UBE3A, EHMT1, one or more genes encoded on chromosome 9q34.3, NPHP1, LIMK1 one or more genes encoded on chromosome 7q11.23, P53, TPI1, FGFR1 and related genes, RA1, SHANK3, CLN3, NF-1, TP53, PFK, CD40L, CYP19A1, PGRN, CHRNA7, PMP22, CD40LG, derivatives thereof, or any combination thereof.

In some embodiments, the nucleic acid can comprise a cDNA that encodes a protein to control or monitor the activity or state of a cell, and/or for assessing the state of a cell. The protein can comprise fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof. The protein can comprise nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof. The protein can comprise a nuclear localization signal (NLS) or a nuclear export signal (NES).

The protein can comprise a CRE recombinase, GCaMP, a cell therapy component, a knock-down gene therapy component, a cell-surface exposed epitope, or any combination thereof. The protein can comprise a chimeric antigen receptor. The protein can comprise a diagnostic agent (e.g., green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof).

In some embodiments, the nucleic acid can comprise a cDNA that encodes a protein for gene editing, or a guide RNA; or a DNA sequence for genome editing via homologous recombination. The protein can comprise a programmable nuclease. In some embodiments, the programmable nuclease is selected from the group comprising: SpCas9 or a derivative thereof; VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof; dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions. The programmable nuclease can comprise a zinc finger nuclease (ZFN) and/or transcription activator-like effector nuclease (TALEN). The programmable nuclease can comprise Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, Cas1, Cas1B, Cas2, Cas3, Cas4, Cas5, Cash, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpfl, C2c1, C2c3, Cas12a, Cas12b, Cas12c, Cas12d, Cas12e, Cas13a, Cas13b, Cas13c, derivatives thereof, or any combination thereof. The heterologous nucleic acid and/or rAAV can comprise a polynucleotide encoding (i) a targeting molecule and/or (ii) a donor nucleic acid. The targeting molecule can be capable of associating with the programmable nuclease. The targeting molecule can comprise single strand DNA or single strand RNA. The targeting molecule can comprise a single guide RNA (sgRNA).

The rAAV disclosed herein can comprise one or more of the heterologous nucleic acids disclosed herein. The heterologous nucleic acid can comprise a polynucleotide encoding a protein. The nucleic acid can be or can encode an RNA agent. The heterologous nucleic acid can comprise a promoter operably linked to the polynucleotide encoding a protein. As disclosed herein, the gene is operatively linked with appropriate regulatory elements in some embodiments. The one or more genes of the heterologous nucleic acid can comprise an siRNA, an shRNA, an antisense RNA oligonucleotide, an antisense miRNA, a trans-splicing RNA, a guide RNA, single-guide RNA, crRNA, a tracrRNA, a trans-splicing RNA, a pre-mRNA, a mRNA, or any combination thereof. The one or more genes of the heterologous nucleic acid can comprise one or more synthetic protein circuit components. The one or more genes of the heterologous nucleic acid can comprise can entire synthetic protein circuit comprising one or more synthetic protein circuit components. The one or more genes of the heterologous nucleic acid can comprise two or more synthetic protein circuits.

The protein can be any protein, including naturally-occurring and non-naturally occurring proteins. Examples include, but are not limited to, luciferases; fluorescent proteins (e.g., GFP); growth hormones (GHs) and variants thereof; insulin-like growth factors (IGFs) and variants thereof; granulocyte colony-stimulating factors (G-CSFs) and variants thereof; erythropoietin (EPO) and variants thereof; insulin, such as proinsulin, preproinsulin, insulin, insulin analogs, and the like; antibodies and variants thereof, such as hybrid antibodies, chimeric antibodies, humanized antibodies, monoclonal antibodies; antigen binding fragments of an antibody (Fab fragments), single-chain variable fragments of an antibody (scFV fragments); dystrophin and variants thereof; clotting factors and variants thereof; cystic fibrosis transmembrane conductance regulator (CFTR) and variants thereof; and interferons and variants thereof.

Examples of protein of interest include, but are not limited to, luciferases; fluorescent proteins (e.g., GFP); growth hormones (GHs) and variants thereof; insulin-like growth factors (IGFs) and variants thereof; granulocyte colony-stimulating factors (G-CSFs) and variants thereof; erythropoietin (EPO) and variants thereof; insulin, such as proinsulin, preproinsulin, insulin, insulin analogs, and the like; antibodies and variants thereof, such as hybrid antibodies, chimeric antibodies, humanized antibodies, monoclonal antibodies; antigen binding fragments of an antibody (Fab fragments), single-chain variable fragments of an antibody (scFV fragments); dystrophin and variants thereof; clotting factors and variants thereof; CFTR and variants thereof; and interferons and variants thereof.

In some embodiments, the protein of interest is a therapeutic protein or variant thereof. Non-limiting examples of therapeutic proteins include blood factors, such as β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (GF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-(3), and the like; soluble receptors, such as soluble TNF-α receptors, soluble VEGF receptors, soluble interleukm receptors (e.g., soluble IL-1 receptors and soluble type II IL-1 receptors), soluble γ/δ T cell receptors, ligand-binding fragments of a soluble receptor, and the like; enzymes, such as -glucosidase, imiglucarase, 3-glucocerebrosidase, and alglucerase; enzyme activators, such as tissue plasminogen activator; chemokines, such as IP-10, monokine induced by interferon-gamma (Mig), Groa/IL-S, RANTES, MlP-l a, MIP-I β, MCP-1, PF-4, and the like; angiogenic agents, such as vascular endothelial growth factors (VEGFs, e.g., VEGF121, VEGF165, VEGF-C, VEGF-2), transforming growth factor-beta, basic fibroblast growth factor, glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-rel easing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); transforming growth factors (TGFs); tissue factors, luteinizing hormone; macrophage activating factors; tumor necrosis factor (TNF); neutrophil chemotactic factor (NCF); nerve growth factor; tissue inhibitors of nietalloproteinases; vasoactive intestinal peptide; angiogenin; angiotropin; fibrin; hirudin; IL-1 receptor antagonists; and the like. Some other non-limiting examples of protein of interest include ciliary neurotrophic factor (CNTF); brain-derived neurotrophic factor (BDNF); neurotrophins 3 and 4/5 (NT-3 and 4/5); glial cell derived neurotrophic factor (GDNF); aromatic amino acid decarboxylase (AADC); hemophilia related clotting proteins, such as Factor IX or Factor X; dystrophin or nini-dystrophm; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GLUT2), aldolase A, f3-enolase, and glycogen synthase; lysosomal enzymes (e.g., beta-N-acetylhexosaminidase A); and any variants thereof.

The protein of interest can be, for example, an active fragment of a protein, such as any of the aforementioned proteins, a fusion protein comprising some or all of two or more proteins, or a fusion protein comprising all or a portion of any of the aforementioned proteins.

In some embodiments, the viral vector comprises a polynucleotide comprising coding regions for two or more proteins of interest, The two or more proteins of interest can be the same or different from each other. In some embodiments, the two or more proteins of interest are related polypeptides, for example light chain(s) and heavy chain(s) of the same antibody.

The protein of interest can be a multi-subunit protein. For example, the protein of interest can comprise two or more subunits, or two or more independent polypeptide chains. In some embodiments, the protein of interest can be an antibody, including, but are not limited to, antibodies of various isotypes (for example, IgG1, IgG2, IgG3, IgG, IgA, IgD, IgE, and IgM); monoclonal antibodies produced by any means known to those skilled in the art, including an antigen-binding fragment of a monoclonal antibody; humanized antibodies; chimeric antibodies; single-chain antibodies; antibody fragments such as Fv, F(ab′)2, Fab′, Fab, Facb, scFv and the like; provided that the antibody is capable of binding to antigen. In some embodiments, the antibody is a full-length antibody. In some embodiments, the protein of interest is not an immunoadhesin.

In some embodiments, the resulting acoustic targeting molecules can be employed in methods and/or therapies relating to in vivo gene transfer applications to long-lived cell populations. In some embodiments, these can be applied to any rAAV-based gene therapy, including, for example: spinal muscular atrophy (SMA), amyotrophic lateral sclerosis (ALS), Parkinson's disease, Friedreich's ataxia, Pompe disease, Huntington's disease, Alzheimer's disease, Battens disease, lysosomal storage disorders, glioblastoma multiforme, Rett syndrome, Leber's congenital amaurosis, chronic pain, stroke, spinal cord injury, traumatic brain injury and lysosomal storage disorders. In addition, rAAVs can also be employed for in vivo delivery of transgenes for non-therapeutic scientific studies such as optogenetics, gene overexpression, gene knock-down with shRNA or miRNAs, modulation of endogenous miRNAs using miRNA sponges or decoys, recombinase delivery for conditional gene deletion, conditional (recombinase-dependent) expression, or gene editing with CRISPRs, TALENs, and zinc finger nucleases.

In some embodiments, the gene encodes immunogenic material capable of stimulating an immune response (e.g., an adaptive immune response) such as, for example, antigenic peptides or proteins from a pathogen. The expression of the antigen may stimulate the body's adaptive immune system to provide an adaptive immune response. Thus, it is contemplated that some embodiments the heterologous nucleic acids provided herein can be employed as vaccines for the prophylaxis or treatment of infectious diseases (e.g., as vaccines).

As described herein, the nucleotide sequence encoding the protein can be modified to improve expression efficiency of the protein. The methods that can be used to improve the transcription and/or translation of a gene herein are not particularly limited. For example, the nucleotide sequence can be modified to better reflect host codon usage to increase gene expression (e.g., protein production) in the host (e.g., a mammal).

The degree of gene expression in the target cell can vary. The amount of the protein expressed in the subject (e.g., the target brain region(s) of the subject) can vary. For example, in some embodiments the protein can be expressed in the subject in the amount of at least about 9 μg/ml, at least about 10 μg/ml, at least about 50 μg/ml, at least about 100 μg/ml, at least about 200 μg/ml, at least about 300 μg/ml, at least about 400 μg/ml, at least about 500 μg/ml, at least about 600 μg/ml, at least about 700 μg/ml, at least about 800 μg/ml, at least about 900 μg/ml, or at least about 1000 μg/ml. In some embodiments, the protein is expressed in the subject in the amount of about 9 μg/ml, about 10 μg/ml, about 50 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1000 μg/ml, about 1500 μg/ml, about 2000 μg/ml, about 2500 μg/ml, or a range between any two of these values. A skilled artisan will understand that the expression level in which a protein is needed for the method to be effective can vary depending on non-limiting factors such as the particular protein and the subject receiving the treatment, and an effective amount of the protein can be readily determined by a skilled artisan using conventional methods known in the art without undue experimentation.

The agent can be an inducer of cell death, for example by a non-endogenous cell death pathway (e.g., a bacterial pore-forming toxin). In some embodiments, the agent (e.g., a protein encoded by a nucleic acid) can be a pro-survival protein. In some embodiments, the agent is a modulator of the immune system. The agent can activate an adaptive immune response, and innate immune response, or both. In some embodiments, the nucleic acid encodes immunogenic material capable of stimulating an immune response (e.g., an adaptive immune response) such as, for example, antigenic peptides or proteins from a pathogen. The expression of the antigen may stimulate the body's adaptive immune system to provide an adaptive immune response. Thus, it is contemplated that some embodiments the compositions provided herein can be employed as vaccines for the prophylaxis or treatment of infectious diseases (e.g., as vaccines). The protein can comprise a CRE recombinase, GCaMP, a cell therapy component, a knock-down gene therapy component, a cell-surface exposed epitope, or any combination thereof. In some embodiments, the protein comprises CFTR, GDE, OTOF, DYSF, MYO7A, ABCA4, F8, CEP290, CDH23, DMD, and ALMS1.

The agent can comprise a non-protein coding gene, such as an RNA agent, e.g., sequences encoding antisense RNAs, RNAi, shRNAs and micro RNAs (miRNAs), miRNA sponges or decoys, recombinase delivery for conditional gene deletion, conditional (recombinase-dependent) expression, includes those required for the gene editing components described herein. The a non-protein coding gene may also encode a tRNA, rRNA, tmRNA, piRNA, double stranded RNA, snRNA, snoRNA, and/or long non-coding RNA (lncRNA). In some embodiments, the RNA agent can comprise non-natural or modified nucleotides (e.g., pseudouridine). In some embodiments, the non-protein coding gene can modulate the expression or the activity of a target gene or gene expression product. For example, the RNAs described herein may be used to inhibit gene expression in a target cell, for example, a cell in the target brain region(s). In some embodiments, inhibition of gene expression refers to an inhibition by at least about 20%, 30%, 40%, 50%, 60%, 70%, 80%, 85%, 90%, 95% and 100%. In some cases, the protein product of the targeted gene is inhibited by at least about 20%, 30%, 40%, 50%, 60%, 70%, 80%, 85%, 90%, 95% and 100%. The gene can be either a wild type gene or a gene with at least one mutation. The targeted protein can be a wild type protein, or a protein with at least one mutation.

Examples of genes encoding therapeutic proteins include a sequence associated with a signaling biochemical pathway, e.g., a signaling biochemical pathway-associated gene or polynucleotide (e.g., a signal transducer). In some embodiments, the methods and compositions disclosed herein comprise knockdown of an endogenous signal transducer accompanied by tuned expression of a protein comprising an appropriate version of signal transducer. Examples of DNA or RNA sequences contemplated herein include sequences for a disease-associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level. Signal transducers can be associated with one or more diseases or disorders. In some embodiments, a disease or disorder is characterized by an aberrant signaling of one or more signal transducers disclosed herein. In some embodiments, the activation level of the signal transducer correlates with the occurrence and/or progression of a disease or disorder. The activation level of the signal transducer can be directly responsible or indirectly responsible for the etiology of the disease or disorder.

The subject in need can be a subject suffering from or at a risk to develop one or more of chronic pain, Friedreich's ataxia, Huntington's disease (HD), Alzheimer's disease (AD), Parkinson's disease (PD), Amyotrophic lateral sclerosis (ALS), spinal muscular atrophy types I and II (SMA I and II), Friedreich's Ataxia (FA), Spinocerebellar ataxia, multiple sclerosis (MS), chronic traumatic encephalopathy (CTE), HIV-1 associated dementia, or lysosomal storage disorders that involve cells within the CNS. The lysosomal storage disorder can be Krabbe disease, Sandhoff disease, Tay-Sachs, Gaucher disease (Type I, II or III), Niemann-Pick disease (NPC1 or NPC2 deficiency), Hurler syndrome, Pompe Disease, or Batten disease.

In some embodiments, the subject is suffering from an acute condition or injury. The subject in need can be a subject suffering from, at risk to develop, or has suffered from a stroke, traumatic brain injury, epilepsy, or spinal cord injury.

Disclosed herein include methods of delivering an agent to one or more target brain region(s) of a subject. In some embodiments, the method comprises: providing an AAV vector comprising an AAV capsid protein disclosed herein, wherein the AAV vector further comprises an agent to be delivered to the one or more target brain region(s); and administering the AAV vector to the subject. The administering can comprise injection(s) into the one or more target brain region(s).

In some embodiments, the method further comprises: administering to the subject an effective amount of an FUS-BBBO agent (e.g., microbubble contrast agent, nanobubble); and applying focused ultrasound (FUS) to the one or more target brain region(s) of the subject.

The agent can be delivered to target brain cell(s) of the target brain region(s) of the subject at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more efficiently than the delivery of the agent to peripheral organs of the subject (e.g., the liver). The AAV vector can be at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more efficient at crossing physically loosened biophysical barriers as compared to the corresponding parental AAV vector that does not comprise the acoustic targeting peptide.

Disclosed herein include methods of treating or preventing a disease or disorder in a subject in need thereof. In some embodiments, the method comprises: administering to the subject an effective amount of a composition disclosed herein (e.g., an AAV capsid protein disclosed herein and an agent to be delivered to the target environment of the subject). The method can comprise: administering to the subject an effective amount of an FUS-BBBO agent (e.g., microbubble contrast agent, nanobubble). The method can comprise: applying focused ultrasound (FUS) to one or more target brain region(s) of the subject, thereby treating or preventing the disease or disorder in the subject.

Disclosed herein include methods of delivering an agent to one or more target brain region(s) of a subject. In some embodiments, the method comprises: administering to the subject an effective amount of a composition disclosed herein (e.g., an AAV capsid protein disclosed herein and an agent to be delivered to the target environment of the subject). The method can comprise: administering to the subject an effective amount of an FUS-BBBO agent (e.g., microbubble contrast agent, nanobubble). The method can comprise: applying focused ultrasound (FUS) to one or more target brain region(s) of the subject, thereby delivering the agent to the target brain region(s).

The method can comprise: administering to the subject an effective amount of an FUS-BBBO agent (e.g., microbubble contrast agent, nanobubble). The method can comprise: applying focused ultrasound (FUS) to one or more target brain region(s) of the subject comprising one or more target brain cell(s), thereby delivering the AAV to the target brain cell(s) to obtain chemogenetically treated target brain region(s) in which target brain cell(s) express the chemogenetic protein. The method can comprise: administering to the subject an effective amount of the corresponding chemical actuator to allow binding of the corresponding chemical actuator or a metabolite thereof with the chemogenetic protein in the target brain cell(s) of chemogenetically treated target brain region(s) to activate or inhibit the target brain cell activity and thereby treat or prevent the disease or disorder in the subject.

The target brain region(s) can comprise one or more target brain cell(s). In some embodiments, applying FUS induces transient blood-brain barrier opening(s) (BBBO) at the target brain region(s). The applying focused ultrasound can be performed at a frequency of 100 kHz to 100 MHz. The applying focused ultrasound can be performed at a frequency of 0.2 to 1.5 MHz. The applying focused ultrasound can be performed within an ultrasound having a mechanical index in a range between about 0.2 and 0.6. Applying FUS to one or more target brain region(s) of the subject can comprise applying one or more FUS pulses to the one or more target brain region(s) over a duration of time. The duration of time (e.g., of the FUS application procedure) can be about 48 hours, about 44 hours, about 40 hours, about 35 hours, about 30 hours, about 25 hours, 20 hours, 15 hours, 10 hours, about 8 hours, about 8 hours, 8 hours, about 7 hours, about 6 hours, about 5 hours, about 4 hours, about 3 hours, about 2 hours, about 1 hour, about 30 minutes, about 15 minutes, about 10 minutes, or about 5 minutes. In some embodiments, the one or more US pulses each have a pulse duration of about 1 hour, about 30 minutes, about 15 minutes, about 10 minutes, about 5 minutes, about 1 minute, about 1 second, or about 1 millisecond.

Applying FUS can induce permeability (blood-brain-barrier opening(s)) of the one or more target brain region(s), optionally for a duration of time after applying FUS of about 8 weeks, about 7 weeks, about 6 weeks, about 5 weeks, about 4 weeks, about 3 weeks, about 2 weeks, about 1 week, about 6 days, about 5 days, about 4 days, about 3 days, about 48 hours, about 44 hours, about 40 hours, about 35 hours, about 30 hours, about 25 hours, 20 hours, 15 hours, 10 hours, about 8 hours, about 8 hours, 8 hours, about 7 hours, about 6 hours, about 5 hours, about 4 hours, about 3 hours, about 2 hours, about 1 hour, about 30 minutes, about 15 minutes, about 10 minutes, or about 5 minutes.

Disclosed herein include methods of controlling a target brain cell activity with respect to a target neural circuit of a subject. In some embodiments, the method comprises: administering to the subject an effective amount of a composition disclosed herein (e.g., an AAV capsid protein disclosed herein and an agent to be delivered to the target environment of the subject), wherein the AAV comprises a nucleic acid encoding a chemogenetic protein under control of a promoter configured to be active in the target brain cell(s), and wherein the chemogenetic protein is configured to activate or inhibit the target brain cell activity following binding with a corresponding chemical actuator or metabolite thereof. The method can comprise: administering to the subject an effective amount of an FUS-BBBO agent (e.g., microbubble contrast agent, nanobubble). The method can comprise: applying focused ultrasound (FUS) to one or more target brain region(s) of the subject comprising one or more target brain cell(s), thereby delivering the AAV to the target brain cell(s) to obtain chemogenetically treated target brain region(s) in which target brain cell(s) express the chemogenetic protein. The method can comprise: administering to the subject an effective amount of the corresponding chemical actuator to allow binding of the corresponding chemical actuator or a metabolite thereof with the chemogenetic protein in the target brain cell(s) of chemogenetically treated target brain region(s) to activate or inhibit the target brain cell activity.

Disclosed herein include methods of modifying a target behavior or physiological function of a subject associated with a target brain cell activity with respect to a neural circuit of the subject. In some embodiments, the method comprises: administering to the subject an effective amount of a composition disclosed herein (e.g., an AAV capsid protein disclosed herein and an agent to be delivered to the target environment of the subject), wherein the AAV comprises a nucleic acid encoding a chemogenetic protein under control of a promoter configured to be active in the target brain cell(s), and wherein the chemogenetic protein is configured to activate or inhibit the target brain cell activity following binding with a corresponding chemical actuator or metabolite thereof. The method can comprise: administering to the subject an effective amount of an FUS-BBBO agent (e.g., microbubble contrast agent, nanobubble). The method can comprise: applying focused ultrasound (FUS) to one or more target brain region(s) of the subject comprising one or more target brain cell(s), thereby delivering the AAV to the target brain cell(s) to obtain chemogenetically treated target brain region(s) in which target brain cell(s) express the chemogenetic protein. The method can comprise: administering to the subject an effective amount of the corresponding chemical actuator to allow binding of the corresponding chemical actuator or a metabolite thereof with the chemogenetic protein in the target brain cell(s) of chemogenetically treated target brain region(s) to activate or inhibit the target brain cell activity and thereby modify the target behavior or physiological function of the subject.

Disclosed herein include methods of treating or preventing a disease or disorder in a subject associated with a target brain cell activity with respect to a neural circuit of the subject. In some embodiments, the method comprises: administering to the subject an effective amount of a composition disclosed herein (e.g., an AAV capsid protein disclosed herein and an agent to be delivered to the target environment of the subject), wherein the AAV comprises a nucleic acid encoding a chemogenetic protein under control of a promoter configured to be active in the target brain cell(s), and wherein the chemogenetic protein is configured to activate or inhibit the target brain cell activity following binding with a corresponding chemical actuator or metabolite thereof.

The systems, methods, compositions, and kits provided herein can, in some embodiments, be employed in concert with the systems, methods, compositions, and kits for described in U.S. Patent Application No. 2019/0175763, entitled, “Methods and systems for noninvasive control of brain cells and related vectors and compositions,” the content of which is incorporated herein by reference in its entirety.

In some embodiments of the compositions, methods and systems herein described are directed to controlling a target brain cell activity with respect to a neural circuit, a behavior, a physiological function and/or a condition associated with a target brain cell activity with respect to a neural circuit of the individual.

In some embodiments, the target brain cell activity indicates a series of biological and biochemical reactions resulting in a direct or indirect effect on the synapses of the neural circuit and related passage of the electrochemical signals. Exemplary target brain cell activity in the sense of the disclosure comprise action potential, intrinsic electroresponsive properties like intrinsic transmembrane voltage oscillatory patterns, and production and/or release of chemicals such as neurotransmitters, gliotransmitters, and additional chemicals identifiable by a skilled person.

A target cell activity with respect to a neural circuit can also be associated with a behavior and/or physiological function of the individual. The wording “associated to” as used herein with reference to two items indicates a relation between the two items such that the occurrence of a first item is accompanied by the occurrence of the second item, which includes but is not limited to a cause-effect relation and sign/symptoms-disease relation.

Accordingly, method to detect a target brain cell activity with respect to a neural circuit comprise not only imaging technique such as PET and fMRI and source-localized EEG, but also behavioral and/or physiological evaluation as will be understood by a skilled person. In research animals the activity a target brain cell activity with respect to a neural circuit can additionally be evaluated through invasive recordings or through histology, as shown previously.

In some embodiments, the activity of a target brain cell activity with respect to the neural circuit is upregulated or downregulated through a specific and selective delivery and expression of chemogenetic protein configured to activate or inhibit the target brain cell activity following binding with a corresponding chemical actuator or metabolite thereof. The wording “chemogenetic protein” refers to a protein having an operative state and inoperative state with respect to the activity of a target brain cell. In some embodiments, chemogenetic receptor in an operative state is configured to react with additional molecules in a cell to provide activation or inhibition of the existing activity of a target brain cell through biochemical reactions.

Exemplary reactions of chemogenetic proteins in an operative state that result in activation or inhibition of an existing activity of a target cell with respect to a neural circuit comprise changes in signaling, transmembrane potential, gene expression that lead to changes in probability of generation of action potentials and/or secretion of chemicals in the target brain cell. These changes can be performed by the chemogenetic protein directly and/or by changing the excitability (probability of activation) of ion channels that induce action potentials, by changing the concentration of ion channels within the cells, or by expressing a new set of ion channels that can achieve the same function as will be understood by a skilled person. For example Non-olfactory G protein-coupled receptors (GPCRs), which are among preferred chemogenetic proteins in the sense of the disclosure, follow the Gq/Gs/Gi pathway which change probability of generation of action potential when expressed in neurons.

In some embodiments, chemogenetic proteins that can be used in methods and systems of the disclosure and related vectors and compositions comprise protein receptors that activate downstream signaling in the cells or gene expression; and ligand-activated ion channels that change the composition of ions inside, and outside of the cell membrane.

Exemplary chemogenetic proteins comprise receptors such as kinases, non-kinase enzymes, G protein-coupled receptors (GPCRs) and ligand-gated ion channels, which can have activating or inhibiting effects on the activity of a target brain cell where they are expressed as will be understood by a skilled person. In some embodiments, chemogenetic proteins suitable in methods and systems of the disclosure and related vectors and compositions comprise DREADDs (hM4Di (inhibitory), hM3Dq (activatory), hM3Ds (activatory), KORD (activatory), PSAM/PSEM ligand activated ion channels (both inhibitory and activatory versions), GluCl (inhibitory), Tetracycline transactivator (changes in gene expression, inhibition), reverse transactivator (changes in gene expression, activation) and others identifiable to a person skilled in the art.

Conversion of a chemogenetic protein from an inoperative state to an operative state can be performed through binding of a corresponding compound also indicated as ligand. The term “corresponding” used in connection with elements such as ligand and chemogenetic protein identify two or more elements capable of reacting one with another under appropriate conditions. Typically, a reaction between corresponding moieties and in particular chemogenetic protein and respective ligand, results in binding of the two elements. The term “bind”, “binding”, “conjugation” as used herein indicates an attractive interaction between two elements which results in a stable association of the element in which the elements are in close proximity to each other.

Attractive interactions in the sense of the present disclosure includes both non-covalent binding and, covalent binding. Covalent binding indicates a form of chemical bonding that is characterized by the sharing of pairs of electrons between atoms, or between atoms and other covalent bonds. For example, attraction-to-repulsion stability that forms between atoms when they share electrons is known as covalent bonding. Covalent bonding includes many kinds of interaction, including σ-bonding, π-bonding, metal to non-metal bonding, agostic interactions, and three-center two-electron bonds. Non-covalent binding as used herein indicates a type of chemical bond, such as protein protein interaction, that does not involve the sharing of pairs of electrons, but rather involves more dispersed variations of electromagnetic interactions. Non-covalent bonding includes ionic bonds, hydrophobic interactions, electrostatic interactions, hydrogen bonds, and dipole-dipole bonds. Electrostatic interactions include association between two oppositely charged entities. An example of an electrostatic interaction includes using a charged lipid as the functional membrane lipid and binding an oppositely charged target molecule through electrostatic interactions. Binding between a chemogenetic actuator and ligand is typically non-covalent bonding which result in the conversion of the chemogenetic protein from an inoperative state to an operative state. The conversion can occur through a conformational change, aggregation or di- or multimerization as will be understood by a skilled person.

In some embodiments, chemogenetic proteins are selected to specifically respond to a class of ligands comprising chemical actuators or metabolites thereof. The wording “chemical actuators” as used herein indicates molecules configured to cross the blood brain barrier of the individual and to convert a chemogenetic protein from an inoperative state to an operative state with respect to the activation or inhibition of a target brain cell activity. Chemical actuators in the sense of the disclosure are typically pharmaceutically inert.

In some embodiments the chemical actuator is configured to directly convert a chemogenetic protein from an inoperative state to an operative state through binding of the chemical actuator with the chemogenetic protein. In some embodiments the binding of the chemical actuator to the chemogenetic protein is specific with respect to the molecules present in the environment where the chemogenetic protein is located. In some embodiments the chemical actuator is configured to indirectly convert the state of a chemogenetic protein from an inoperative state to an operative state through binding of a metabolite of the chemical actuator with the chemogenetic protein. The term metabolite indicates a molecule that can be obtained through breakdown of chemical bonds by enzymes, thermal degradation, or conjugation/binding of a reference molecule with molecules already present in the body. In some embodiments the binding of the metabolite with the chemogenetic protein is specific with respect to the molecules present in the environment where the chemogenetic protein is located.

The wording “specific” “specifically” or “specificity” as used herein with reference to the binding of a first molecule to second molecule refers to the recognition, contact and formation of a stable complex between the first molecule and the second molecule, together with substantially less to no recognition, contact and formation of a stable complex between each of the first molecule and the second molecule with other molecules that may be present. Exemplary specific bindings are antibody-antigen interaction, cellular receptor-ligand interactions, polynucleotide hybridization, enzyme substrate interactions etc. The term “specific” as used herein with reference to a molecular component of a complex, refers to the unique association of that component to the specific complex which the component is part of. By “stable complex” is meant a complex that is detectable and does not require any arbitrary level of stability, although greater stability is generally preferred.

In some embodiments of the disclosure chemical actuators and related metabolite refers to molecules which in itself are not naturally present or are present but are biologically inert with respect to the target brain cell not expressing the corresponding chemogenetic protein at the concentrations required for the chemogenetic protein to activate or inhibit the target cell activity when in an operative state.

In some embodiments, a chemical actuator or related metabolite configured to bind the chemogenetic receptor can be a molecule that cross through the BBB based on the lipid-mediate free diffusion. Those molecules can have a molecular weight below 500 Daltons, a number of hydrogen bonds lower than and low affinity (Ku higher than 10 micromolar) to efflux pumps such pGp. An example of these molecule is clozapine, can activate chemogenetic receptors of DREADD class at doses >10-fold below what is typically used in the clinic, and consequently has limited side effects.

In some embodiments, a chemical actuator or related metabolite configured to bind the chemogenetic receptor can be a molecule that is a conjugate of another molecule present in the body that naturally cross the BBB, such as amino acids or hexoses. A conjugate refers to a compound formed by the joining of two or more chemical compounds. Examples of these molecules have a binding affinity to GLUT1 and LAT1 transporters.

In some embodiments, a chemical actuator or related metabolite configured to bind the chemogenetic receptor can be a small molecule configured to cross the Blood Brain Barrier (BBB) through active transport by the transporters present in the BBB. Exemplary chemical actuators or related metabolite having these features includes α-amino acids that have a binding affinity to LAT1, LAT2, transporter (e.g. melphalan), or molecules that place the amino- and carboxyl-groups within 0.4 nm radius of the relative positions of these two functional groups in α-amino acids (e.g. gabapentin) in the solution structure of the molecule. Exemplary chemical actuator or related metabolite having these features also include beta-amino acids and conjugates which cross the BBB through pathways analogous to transport of beta-alanine, as well as other conjugates of amino acids, which are actively transported through the BBB which are configured for entering the BBB.

In some embodiments, a chemical actuator or related metabolite configured to bind the chemogenetic receptor can be a fatty acid or a conjugate thereof, which is configured to crosses the BBB through fatty acid transporter.

In some embodiments, a chemical actuator or related metabolite configured to bind the chemogenetic receptor can be a protein/peptide therapeutics that cross the BBB. Exemplary chemical actuators or related metabolite having these features include conjugates or protein fusions of antibody (or antibody-fragments) targeting endogenous protein transporters that are present in the BBB (E.g. TfR, PepT1, PepT2, Oatp2, OAT-K1, OATP) and allow trans-BBB transport. Exemplary chemical actuators or related metabolite having these features further include molecules exhibiting affinity to the endogenous protein transporters present in the BBB, e.g. peptides evolved by directed evolution, or through in silico protein engineering methods.

In some embodiments, a chemical actuator or related metabolite configured to bind the chemogenetic receptor can be a molecule passing through the BBB using transcytosis of engineered immunoglobulin or fusion proteins that bind to receptors present in the BBB. In some embodiments, a chemical actuator or related metabolite configured to bind the chemogenetic receptor can be a molecule native in species other than the species of the individual, e.g. salvinorin A is a natural product that can be used to activate KORD receptor in mammals. In some embodiments, a chemical actuator or related metabolite configured to bind the chemogenetic receptor can be molecules that have been engineered to specifically bind with a corresponding chemogenetic proteins. In some embodiments a chemical actuator or related metabolite configured to bind the chemogenetic receptor can be deliverable through a number of routes, such as oral administration or injections intravenously, subcutaneously, intramuscularly or intraperitoneally.

Table 1 provides a list of exemplary chemogenetic proteins and corresponding chemical actuators that can activate the chemogenetic proteins through direct specific binding to the chemogenetic protein.

TABLE 1
Exemplary Chemogenetic Proteins and Corresponding Chemical Actuators

| | | |
|---|---|---|
| Name | Protein(s) | Ligand |

Representative kinases

| | | |
|---|---|---|
| Allele-specific kinase | v-I388G | Compound 3g |
| inhibitors | | |
| Analogue-sensitive | v-Src (I338G, v-Src-as1), c-Fyn | K252a and PPI analogues |
| kinases | (T339G, c-Fyn- as1), c-Ab1 | |
| | (T315A, c- Ab1-as2), CAMK Ilα | |
| | (F89G, CAMK llα-as1) and CDK2 | |
| | (F80G, CDK2-as1) | |
| Rapamycin-insensitive | TORC2 V2227L | BEZ235 |
| TOR complex 2 | | |
| ATP-binding pocket | Ephb1^{T697G}, Ephb2rn^{T699A}, and | PP1 analogues |
| mutations in EphB1, | Ephb3^{T706A} | |
| EphB2 and EphB3 | | |
| ATP-binding pocket | TrkA^{F592A}, TrkB^{F616A}, and | 1NMPP1 and 1NaPP1 |
| Mutations of TrkA, | TrkC^{F617A} | |
| TrkB and TrkC | | |

Representative Enzymes

| | | |
|---|---|---|
| Metalloenzymes | Achiral biotinylated rhodium- | |
| | diphosphine complexes | |
| Engineered | Chemically conjugating a | Enhanced activity |
| transaminases | pyridoxamine moiety within the | |
| | large cavity of intestinal fatty acid | |
| | binding protein | |

Representative GPCRs

| | | |
|---|---|---|
| Allele-specific GPCRs | β2-adrenergic receptor, D113S | 1-(3′,4′ - |
| | | dihydroxyphenyl)-3- |
| | | methyl-L-butanone (L- |
| | | 185,870) |
| RASSL-Gi (receptors | k-opioid chimeric receptor | Spiradoline |
| activated solely b | | |
| synthetic ligand) | | |
| Engineered GPCRs | 5-HT2A serotonin receptor | Ketanserin analogues |
| | F340→L340 | |
| Gi-DREADD | M2- and M4 mutant muscarinic | Clozapine-N-Oxide |
| | receptors | |
| Gq-DREADD | M1, M3, and M5- mutant | Clozapine-N-oxide |
| | muscarinic receptors | |
| Gs-DREADD | Chimeric M3-frog Adrenergic | Clozapine-N-oxide |
| | receptor | |
| Arrestin-DREADD | M3Dq R165L | Clozapine-N-oxide |
| Axonally-targeted | hM4d-neurexin variant | Clozapine-N-oxide |
| silencing | | |
| KORD | k-opioid receptor D138N mutant | Salvinorin B |

Representative Channels

| | | |
|---|---|---|
| GluC1 | Insect Glutmate chloride channel; | Ivermectin |
| | Y182F mutation | |
| TrpV1 | TrpV1 in TrpV1 KO mice | capsaicin |
| PSAM | Chimeric channels PSAM^{Q79G, L141S} | PSEM^{9S} |
| PSEM | PSAM-GlyR fusions | PSEM^{89S}; PSSEM^{22S} |

In some embodiments, chemogenetic receptors can be engineered to modify the related binding selectivity to minimize the binding affinity with their native ligand and maximize affinity for another chemical actuator non native to the individual. For example, a native human muscarinic receptor (hM3, human muscarinic receptor 3) can be engineered into a chemogenetic receptor (DREADD), through mutations introduced to change its binding affinity from acetylcholine to clozapine-n-oxide. At the same time, the engineered receptor can be tested against other ligands present in the mouse brain to ascertain that no endogenous ligands in the brain will lead to activation of the receptor. In another example, a receptor from a different species (e.g. GluCl) can respond to an available drug that does not have significant effect in a mammalian system.

In some embodiments, chemogenetic proteins comprise Designer Receptor Exclusively Activated by Designer Drugs or DREADDs. DREADDs are modified versions of natural activatory or inhibitory GPCRs, engineered to respond to synthetic molecules rather than endogenous ligands. DREADDs have been considered for clinical translation due to their ability to selectively control neural circuits.

DREADDs can be classified as Gi-based DREADDs (Gi-DREADDs), Gq-based DREADDs (Gq-DREADDs), and Gs- and β-Arrestin-DREADDs. Exemplary DREADDs include hM3Dq, hM4Di, GsD, R165L β-Arr DREADD, hM4D^{nr×n}, KORD (κ-opioid-derived DREADD) and others identifiable to a person skilled in the art. Detailed information about various chemogenetic receptors and particularly DREADDs can be found in the review article by Roth B. L. which is incorporated by reference in its entirety.

For example, Gi-DREADDs include hM2Di, hM4Di, and KORD. hM2Di and hM4Di can be activated by chemical actuators such as clozapine-N-oxide (CNO), DREADD agonist 21, and perlapine. KORD can be activated by pharmaceutically inert compound such as salvinorin B. Both hM4Di and KORD inhibit neuronal activity via two mechanisms: (a) induction of hyperpolarization by Gβ/γ-mediated activation of G-protein inwardly rectifying potassium channels (GIRKs) and (b) via inhibition of the presynaptic release of neurotransmitters (e.g., synaptic silencing). Gq-DREADDs include hM1Dq, hM3Dq and hM5Dq DREADD. Gq-DREADD can be activated by chemical actuators such as CNO, a pharmacologically inert metabolite of the atypical antipsychotic drug clozapine. Gs-DREADDs are created by swapping the intracellular regions of the turkey erythrocyte β adrenergic receptor for equivalent regions of a rat M3 DREADD to create a rat eGs-DREADD. β-Arrestin-DREADDs are DREADDs signaling exclusively via β-arrestin.

The chemogenetic protein, for example a hM4Di DREADD, or hM3Dq DREADD can change activity of cells in which it is expressed upon exposure to a drug. Such activity can be defined as any perturbation of signaling, transmembrane potential, gene expression, or molecular composition of the cell. Other chemogenetic proteins can be ligand-activated ion channels (such as PSEM/PSEM, or ivermectin responsive GluCl), or a drug-activated transcription factor, such as tetracycline transactivator (tTA).

In some embodiments, selection of a specific chemogenetic receptor is performed based on the desired effect on a target brain cell activity with respect to a target neural circuit and in some embodiments whether an activation or inhibition of the target brain cell activity is desired.

In some embodiments, where an inhibition of the target brain cell activity with respect to a target neural circuit is desired, an inhibitory GPCR coupled to Gi g-protein, such as hM4Di DREADD, an inhibitory ion channel that leads to decrease of likelihood of depolarization, such as GluC, or other receptors that lead to decreased likelihood of neuronal activation, such as through depolarization of the membrane, can be selected.

In embodiments where an activation or increase of a target brain cell activity with respect to a target neural circuit is desired, an activatory GPCR coupled to Gq or Gs g-protein, such as hM3Dq or activatory ion channel that leads to increase of likelihood of depolarization of the membrane, such as Ca²⁺ conducting PSAM/PSEM can be selected.

In some embodiments, selection of an activating or inhibiting chemogenetic protein can be performed to obtain a target behavior or physiological function of an individual and/or to treat or prevent the condition in the individual associated with the activity of the target brain cell with respect to the target neural circuit of the individual, as will be understood by a skilled person. For example, to reduce the activity of an overactive region of the brain, such as an epileptogenic focus in epilepsy, an inhibiting chemogenetic protein would be chosen and targeted to excitatory cells within the epileptogenic focus.

In methods and system herein described administering a chemogenetic protein configured to activate or inhibit, when in an operative state, a target brain cell activity with respect to a target neural circuit, is performed by acoustically delivering to the target controlling brain cell of the individual an AAV configured to express in the target controlling brain cell a gene encoding for a chemogenetic protein to obtain a chemogenetically treated target brain cell comprising an expressed chemogenetic protein.

In some embodiments, the acoustically delivering is performed by (i) applying focused ultrasound to a target region in the brain of a subject and systemically administering an effective amount of microbubble contrast agents designed to stably cavitate in response to the ultrasound field produced by the focused ultrasound at the target brain region for a time and under conditions to induce transient blood-brain barrier opening; and (ii) before, simultaneously, in combination with and/or after applying focused ultrasound, systemically administering an effective amount of an AAV vector provided herein configured to enter the brain at the site of an open blood-brain barrier and deliver to the brain cells at that site a gene encoding a chemogenetic protein under the control of a promoter active in the target brain cell, the chemogenetic protein configured to activate or inhibit the target brain cell activity following binding with a corresponding chemical actuator or metabolite thereof.

The term “ultrasound” refers to sound with frequencies higher than the audible limits of human beings, typically over 20 kHz. Ultrasound devices typically can range up to the gigahertz range of frequencies, with most medical ultrasound devices operating in the 0.2 to 18 MHz range. The amplitude of the waves relates to the intensity of the ultrasound, which in turn relates to the pressure created by the ultrasound waves. Applying ultrasound can be accomplished, for example, by sending strong, short electrical pulses to a piezoelectric transducer directed at the target. Ultrasound can be applied as a continuous wave, or as wave pulses as will be understood by a person skilled in focused ultrasound.

Focused ultrasound (“FUS”) refers to the technology that uses ultrasound energy to target specific areas of a subject, such as a specific area of a brain or body. FUS focuses acoustic waves by employing concave transducers that usually have a single geometric focus, or an array of ultrasound transducer elements which are actuated in a spatiotemporal pattern such as to produce one or more focal zones. At this focus or foci most of the power is delivered during sonication in order to induce mechanical effects, thermal effects, or both. The frequencies used for focused ultrasound are in the range of 200 KHz to 8000 KHz. Depending on the design of the ultrasound transducers and the ultrasound parameters, the target brain region can be as in a range between 1 and 10 mm in diameter as will be understood by a skilled person.

In some embodiments, the applying focused ultrasound can be performed by performing FUS-BBBO. The term “FUS-BBBO” refers to techniques that applies ultrasound waves to a target region, in conjunction with microbubbles, to temporally induce localized blood-brain barrier (“BBB”) opening noninvasively and regionally. In some embodiments, FUS delivers low frequency ultrasound waves which cause mechanical oscillations in microbubbles resulting in disruption of endothelial cells (“EC”) tight junctions leading to enhanced BBB permeability to agents. FUS-BBBO has also been tested in the clinic as will be understood by skilled person.

Accordingly, in methods and systems of the present disclosure, the applying of a focused ultrasound is performed together with the systemic administration to the individual of an effective amount of microbubble contrast agents designed to stably cavitate in response to an ultrasound field produced by the focused ultrasound at the target brain region, for a time and under conditions allowing to obtain a transient BBB opening,

In some embodiments the applying ultrasound can be performed before, simultaneously, or in combination administration of a microbubble contrast agent according to any settings that will ensure application of ultrasound in presence of an effective amount of microbubbles at the BBB of the individual in correspondence with the target region.

The term “contrast agent” refers to an agent (material) in aqueous media, including water, saline, buffer, liquid media, configured to increase contrast in ultrasound methods. By an increase in contrast, it is meant that the differences in image intensity between adjacent tissues visualized by an ultrasound imaging method are enhanced. The contrast agent can be provided in any pharmaceutically and/or physiologically suitable liquid or buffer known in the art. For example, the contrast agent can be contained in water, physiological saline, balanced salt solutions, buffers, aqueous dextrose, glycerol or the like. In certain embodiments, the contrast agent can be combined with agents that can stabilize and/or enhance delivery of the contrast agent to the target site. For example, the contrast agent can be administered with detergents, wetting agents, emulsifying agents, dispersing agents or preservatives.

The FUS-BBBO agent (e.g., microbubble contrast agent, nanobubble) can comprise an encapsulated gas and a shell of lipid or protein. The encapsulated gas can be perfluren or C3F8. The FUS-BBBO agent (e.g., microbubble contrast agent, nanobubble) can be selected from the group comprising nanobubbles, Definity®, Sonovue®, Optison®, and USphere®. The FUS-BBBO agent (e.g., microbubble contrast agent, nanobubble) can have an average diameter of between about 10 nm and 1000 nm or between about 1 and 5 microns, optionally the microbubble contrast agent is a nanobubble. The FUS-BBBO agent (e.g., microbubble contrast agent, nanobubble) and the composition can be co-administered. In some embodiments, the microbubble contrast agents are nanobubbles. In some embodiments, nanobubbles are below 1 micron diameter. The method can comprise: administering a chemical actuator to the subject. Administering of the chemical actuator can be performed at least one week after the administrating of the composition and the applying of focused ultrasound. The chemical actuator can be selected from the group comprising salvinorin B, KORD Dreadd, deschloroclozapine, clozapine-N-oxide, clozapine, compound 21, and perlapine.

Nanobubbles and other FUS-BBBO agents are described in Cheng et al (Cheng, Bingbing, Chenchen Bing, and Rajiv Chopra. “The effect of transcranial focused ultrasound target location on the acoustic feedback control performance during blood-brain barrier opening with nanobubbles.” Scientific reports 9.1 (2019): 1-10.), the content of which is incorporated herein by reference in its entirety.

The contrast agent comprised in methods and systems of the disclosure comprises “microbubbles” defined as particles smaller than the blood vessel diameter and able to undergo stable cavitation or capable of inducing stable cavitation. In some embodiments microbubble contrast agents in the sense of the disclosure comprises microbubbles designed to stably cavitate in response to an ultrasound field produced by the focused ultrasound at the target brain region. Microbubbles can comprise an inert gas encapsulated by a shell. The microbubbles in general have an average diameter between 1 and 5 microns.

Exemplary microbubbles include The Definity®, Sonovue®, Optison®, and USphere®. Such microbubbles contain an encapsulated gas (typically perfluren, C3F8) and a shell. This shell depends on the manufacturer and can be either lipid or protein. For example, definity contains a mixture of DPPA, DPPC, MPEG5000 DPPE lipids in the shell. Optison has a shell made out of albumin. Sonovue has a phospholipid shell and USphere, is made of lipid and polymeric adducts.

Microbubbles are typically administered intravenously to the individual for a time and in an effective amount to achieve a concentration in the target brain region causing in combination with ultrasound waves mechanical oscillations disruption of tight junctions between endothelial cells (“EC”) without disrupting the cells leading to enhanced BBB permeability. In some embodiments the timing of contrast agent depends on the half life of microbubbles in the organism of the given individual as will be understood by a skilled person.

In some embodiments, the microbubbles are typically administered to the target region before the application of focused ultrasound at a time sufficient to provide the microbubbles to the BBB at appropriate concentrations. Typically, the microbubbles are administered between 0 and 1 minutes before the application of focused ultrasound. In some cases, the microbubbles are administered first, followed by an immediate application of focused ultrasound.

In some exemplary embodiments, wherein the FUS is applied about 10 seconds after administering of the contrast agent, the microbubble concentration can be in the range of 1E5-1E7 microbubbles per g of body weight for mice; 2.4E7-2.4E9 microbubbles/kg of body weight for rats, and 1.2E7-1.2E9 per kg of body weight for non-human primates and humans. In some preferred embodiments, the microbubble concentration is 1.5E9 microbubbles/kg of body weight in mice, 2.4E8 per kg of body weight in rats, and 1.2E8 per kg of body weight in non-human primates and humans.

Additional combinations of timing and concentrations of the administering of a contrast agent according to methods of the instant disclosure can be identified by a skilled person taking into account that the presence of microbubbles present in the blood supply allows for the reduction of the ultrasound intensity that is necessary for BBB opening, the containment of most of the disruption within the vasculature, and the reduction of the likelihood of irreversible neuronal damage. In this connection, increase of the concentration of contrast agent will allow increase of the time interval before applying the focused ultrasound according to methods of the disclosure. For example, increase of the concentration of the contrast agent by 10-times, allows one to wait 5 minutes and still have the same BBB opening as one would have by injecting 1/10^{th} and waiting 10 seconds.

Accordingly, in some embodiments, the applying focused ultrasound to a target region in the brain of an individual and systemically administering to the individual an effective amount of microbubble contrast agents is performed to temporally induce blood-brain barrier opening. The term “transient” “temporary” refers to a reversible opening for a limited period of time before the blood-brain barrier returning to its initial state.

In some embodiments, applying focused ultrasound to a target region in the brain can transiently or temporarily open the blood-brain barrier (“BBB”) in the target region to allow the delivery of an effective amount of vector. The term “BBB” refers to a highly selective semipermeable border that separates the circulating blood from the brain and extracellular fluid in the central nervous system. BBB allows the passage of water, some gases, and lipid-soluble molecules by passive diffusion, as well as the selective transport of small molecules such as glucose and amino acids that are crucial to neural function, but restricts the diffusion of solutes in the blood and large or hydrophilic molecules into the cerebrospinal fluid. As such, the BBB is able to prevent the entrance of most substances such as toxins, drugs, viruses and bacteria from the blood stream into brain tissue. Due to the BBB's restrictive permeability, the BBB presents a natural barrier for the delivery of gene vectors to the brain.

In some embodiments, the subject is placed in a direct contact with an ultrasound-conductive medium, such as ultrasound gel or degassed water, that is coupled to an ultrasound transducer. The transducer is focused on the area of intended opening of the BBB. If needed, multiple transducers or array transducers can be used to correct for aberration in the sound field due to the skull or vertebrae. The targeted region is chosen based on medical imaging and/or anatomical information of the subject. Such imaging includes, but is not limited to, MRI, CT, PET, ultrasound imaging. Anatomical information will use external fiducial markers on the body to establish location of the targeted site.

The sites and cells targeted depend on various applications and the intended effect. Different brain regions perform different functions and therefore neuromodulation of different brain regions with ATAC can lead to different behavioral/therapeutic/cell-activity effects. For example, for epilepsy treatment such site can be a seizure focus area. While for memory-related disorders, it can be a hippocampus. And for treatment of Parkinson's disease, it can be the basal ganglia.

In some embodiments, single- and/or multi-element ultrasound transducers operating at frequencies of 100 kHz to 100 MHz are used. The preferred ultrasound frequency range is between 1 and 10 MHz for rats and mice, and 0.2 to 1.5 MHz for non-human primates and humans. The transducers are typically driven by a waveform generator and radiofrequency amplifier. Accounting for attenuation through the medium, brain tissue and bone, the acoustic output power at the transducer focus is sufficient to open the BBB after infusion of microbubble contrast agents and mechanical index is kept below 1.9, and above 0.2. The preferred range of mechanical indices of ultrasound at the brain is between 0.2 and 0.6 for all species. The term “mechanical index” is a measure of acoustic power, i.e. the amount of acoustic energy per time unit. Acoustic power shows the amplitude of the pulse pressure of the ultrasound beam. Mechanical index provides information about the magnitude of energy administered to a subject during the ultrasound application.

In some embodiments, the AAV carries a gene encoding for a chemogenetic protein acoustically delivered to a target brain region comprises the gene encoding a chemogenetic protein under control of a promoter configured to be active in the target brain cell. In some embodiments, the nucleic acids encoding the chemogenetic protein can be under the control of a cell specific promoter operatively connected to the gene of the chemogenetic protein. A “promoter” as used herein indicates is a region of DNA that initiates transcription of a particular gene as will be understood by a skilled person. In embodiments wherein cell specific promoter of the vector are selected to be cell specific, the vectors is configured to have the promoter controlling the expression of the chemogenetic gene, specifically recognized by the native synthetic machinery of the target controlling brain cell. In some embodiments, wherein the target brain cell is a neuron, the cell specific promoter is a synapsin, such as synapsin1 and in some embodiments human synapsin1. Other variants of Synapsin 1 promoter exist (eg. Rat synapsin 1 promoter) which are closely associated and identifiable to a skill person. In some embodiments, a neuron cell specific promoter can be a tyrosine hydroxylase promoter, a melanopsin promoter, or a promoter that expresses in retinal neurons. In another embodiment, the neuron cell specific promoter can be a PRSx8 promoter which specifically targets catecholaminergic neurons. PRSx8 is based on an upstream regulatory site in the human Dopamine Beta-Hydroxylase (“DBH”) promoter and drives high levels of expression in adrenergic neurons. In another embodiment, the neuron-specific promoter can be preprotachykinin-1 promoter (TAC-1). Other exemplary cell-specific promoters include neuron-specific enolase (NSE) and the promoters listed in the following Table 2.

TABLE 2
Exemplary Cell-specific Promoters

| Name | Size | | Specificity |
|---|---|---|---|
| GFAP104 | 845 | bp | Hybrid of EF1a and GFAP |
| CamKlla | 1.2 | kb | Specific expression in excitatory neurons in the neocortex and |
| | | | hippocampus |
| CK0.4 | 217 | bp | Calcium/Calmodulin-dependent kinase II alpha |
| GFAP | 2.0 | kb | Specific in astrocyte |
| MBP | 1.3 | kb | Myelin basic protein promoter, efficient transduction of |
| | | | oligodendrocytes by adeno-associated virus type 8 vectors |
| Synapsin | 471 | bp | Specific in neuron |
| Mecp2 | 230 | bp | Truncated Mcep2 neuron specific |
| c-fos | 1.7 | kb | Activity-dependent promoter |
| Somatostat | 1.2 | kb | Restricting expression to GABAergic neuron |
| Rpe65 | 700 | bp | Retinal Pigment epithelium-specific expression in vivo and in vitro |
| NSE | 1.3 | kb | Neuron-specific enolase promoter |

Additional promoters specific for any target neurons and/or glial cells can be identified by a skilled person e.g. by sequencing transcriptome of selected group of cells in model organisms (e.g. mouse) and finding specific genes that are expressed in that cell line, producing cell-specific proteins (CSP). One would then perform a computational search of sequences resembling promoters and enhancers upstream of that gene on the DNA, package that sequence along with a reporter gene (RG) in a viral vector. For each candidate sequence one would use an antibody against a cell-specific protein (CSP) to confirm identity of the cells in histology and confirm that expression of a reporter gene (RG) is restricted only to cells that also contain CSP. Such procedure can be understood by a person skilled in the art.

AAV vectors herein described can further comprise additional regulatory sequences that can be also cell specific. Accordingly, in some embodiments, an AAV vector of the disclosure can comprise a polynucleotide encoding for one or more chemogenetic proteins herein described, under control of one or more regulatory sequence regions in a configuration allowing to express chemogenetic proteins encoded by the polynucleotide in presence of suitable cellular transcription and translation factors.

Regulatory regions of a gene herein described comprise transcription factor binding sites, operators, activator binding sites, repressor binding sites, enhancers, protein-protein binding domains, RNA binding domains, DNA binding domains, silencers, insulators and additional regulatory regions that can alter gene expression in response to developmental and/or external stimuli as will be recognized by a person skilled in the art.

The AAV vector can comprise one or more polynucleotides encoding a chemogenetic protein herein described under control of one or more regulatory sequences including enhancer regions in a configuration allowing regulation of expression of the chemogenetic proteins encoded by the polynucleotide in presence of necessary cellular transcription and translation factors. The regulatory sequences such as promoter and/or enhancer regions can be arranged proximally and/or distally 5′ and/or 3′ to the one or more polynucleotides encoding for a chemogenetic protein herein described. The AAV vector can also comprise additional regulatory elements such as ribosome binding sites, and transcription termination sequences. In some embodiments, the regulatory sequences of promoter and/or enhancer regions regulating expression of one or more polynucleotides encoding a temperature sensitive transcription factors comprise DNA regulatory region regulated by binding of one or more temperature sensitive transcription factors.

In embodiments, wherein in addition to a cell specific promoter, one or more regulatory regions of the vector are selected to be cell specific, the vectors is configured to have the promoter and the regulatory regions controlling the expression of the chemogenetic gene, specifically recognized by the native synthetic machinery of the target brain cell.

For example, the AAV vector and/or promoter can be selected to target controlling brain cell in hippocampus, which control formation of memory and can contribute to beginning of seizure activity, similarly area of temporal cortex and amygdala can also contribute to formation of seizures and reducing activity of activatory neurons (CamkIIa promoter) or inducing activity of inhibitory neurons (Parvalbumin positive neurons) can bring reduction of seizure frequency and severity. In dopaminergic cells in basal ganglia, upregulation of dopaminergic cell (tyrosine hydroxylase promoter) activity can be beneficial in treatment of Parkinson's disease. The same dopaminergic cells in ventral tegmental area can be activated to improve outcomes of mood disorders.

In some embodiments, wherein the target brain region is of microns dimensions the AAV vector can comprise one or more regulatory regions which are in series with regulatory regions of the target brain cell in accordance with an intersectional approach. The term “in series” as used herein refers to a connection between a regulatory regions and related regulatory molecule through biochemical reactions along a single linear circuit path. An ‘in-series’ arrangement requires presence and activity of both regulatory regions independently activated or repressed in temporal succession, to obtain expression of the chemogenetic protein in the target brain cell.

Accordingly, in embodiments performed with an intersectional approach, the expression of the AAV vector encoding the chemogenetic protein can be controlled by the activation or inhibition of at least another molecular component through direct or indirect reaction of the at least another molecular component with the AAV vectors herein described.

In some embodiments, the individual is a transgenic animal engineered to express regulatory regions and/or corresponding molecules. In a representative example, the transgenic animal can be a transgenic mice expressing Cre recombinase under a cell-specific promoter, to achieve cell-specific and circuit-specific expression of DREADDs. The Cre-recombinase system allows for the expression of a gene following delivery with a Cre-dependent viral vector only in cells expressing. In some embodiments transfection can be performed unilaterally within the brain in a small cell population located deep within the midbrain and thus difficult to reach with traditional invasive approaches, and performed FUS mediated BBB opening applied to the midbrain concomitant with injection of a Cre-dependent AAV9-encoded hM3receptor (muscarinic receptor M3). The virus induced the activation of tyrosinehydroxylase-positive dopaminergic neurons of the midbrain in tyrosine hydroxylase (TH)-Cre transgenic mice following peripheral administration of CNO. After confirming successful viral infection by mCherry immunofluorescence, activation of the targeted neurons was measured by imaging c-Fos expression, an immediate early gene linked to neuronal activity that is commonly used as a marker of cellular activation. ATAC increased c-Fos activation CRE-expressing strain with the CRE activities restricted to one or more specific areas of the brain region.

Embodiments performed with an intersectional approach allow delivery and expression of chemogenetic protein in target brain region of micrometer size.

The detection of expression of the AAV vector can be achieved in vivo by introducing a chemogenetic ligand that has been radiolabeled and imaging it with positron emission tomography. This approach yields information about both quantitative levels of expression and the ability of a receptor to bind a specific ligand. In some individual such as research animals, immunostaining can be used to evaluate expression and proper subcellular localization of the receptors. In some embodiments positive immunostaining is indicative that the genetic material encoding the chemogenetic protein has been successfully delivered to the cell. or other detection techniques.

In general, in some embodiments, the administration of the AAV vector is performed to have a presence of vectors carrying genetic material in the blood concurrently with the occurrence of the BBB opening. In some embodiments, the timing of the administration of the vectors with respect to the ultrasound application depends on the serum half-lives of the vectors. For vectors with short serum half-lives, the ultrasound application is performed within 10 minutes of the administration of gene delivery vectors. The injection can be performed either shortly before, or after, the focused ultrasound procedure. In some embodiments, the microbubbles and vectors are co-injected within 1 minute before the focused ultrasound procedure. Vectors with long serum half-lives can be injected longer than 10 minutes before the FUS-BBBO procedure.

In some embodiments, the administration of an AAV vector encoding a chemogenetic protein to the target brain region can be simultaneously combined with or in sequential of the administration of the microbubbles.

In some embodiments, acoustically delivering to a target brain cell an AAV vector herein described to provide a chemogenetically treated target brain region is performed in combination with administering to the individual a chemical actuator configured to switch the expressed chemogenetic protein conformation into the operative state.

In the method, the administering of the chemical actuator is performed for a time and under condition to allow binding of the chemical actuator or of a metabolite thereof with the expressed chemogenetic protein in the target brain cell of the chemogenetically treated brain region, and activation or inhibition of the target brain cell activity through stimulation of the target brain cell by the expressed chemogenetic protein in the operative state

In some embodiments the chemical actuator or metabolite thereof can be configured to be able to enter the brain from blood stream through BBB via a direct passage or via chemical alteration such as metabolism or prodrug conversion processes and then bind to the chemogenetic proteins. Examples of chemical actuators include CNO, compound 21, perlapine, clozapine or others identifiable to a person skilled in the art.

In some embodiments, chemical actuators are configured to cross through the BBB based on the lipid-mediated free diffusion. These drugs typically have a molecular weight below 500 Daltons and have fewer than 10 hydrogen bonds. An example of such molecule is clozapine, which at low doses has limited side effects and can activate chemogenetic receptors of DREADD class.

In some embodiments, chemical actuators can be conjugates of molecules present in the body that naturally cross the BBB, such as amino acids or hexoses. Exemplary chemical actuators in this class include molecules having a binding affinity to GLUT1 and LAT1 transporters. The conjugates of a given molecule are defined as molecules having identical structures of the given molecule with exception of at least one atom or bond which are used to connect to another molecule.

Chemical actuators also include small molecule drugs capable of crossing the BBB through active transport by the transporters present in the BBB. Exemplary chemical actuators in this class include α-amino acids having a binding affinity to LAT1, LAT2, transporter (e.g. melphalan), or molecules that place the amino- and carboxyl-groups within 0.4 nm radius of the relative positions of these two functional groups in α-amino acids (e.g. gabapentin) in the solution structure of the molecule. Other exemplary chemical actuators include beta-amino acids and conjugates capable of crossing the BBB through pathways analogus to transport of beta-alanine, as well as other conjugates of amino acids that are actively transported through the BBB.

In some embodiments, chemical actuators can be fatty acids and their conjugates capable of crossing the BBB through fatty acid transporter as well as molecules passing through the BBB using transcytosis of engineered immunoglobulin or fusion proteins that bind to receptors present in the BBB.

Additional chemical actuators include protein or peptide therapeutics capable of crossing the BBB. Exemplary chemical actuators in this class include conjugates or protein fusions of antibody or antibody-fragments targeting endogenous protein transporters that are present in the BBB, such as TfR, PepT1, PepT2, Oatp2, OAT-K1, OATP, and allow trans-BBB transport. Additional examples include other binding agents exhibiting affinity to the endogenous protein transporters present in the BBB, such as peptides evolved by directed evolution, or through in silico protein engineering methods.

Chemical actuators once administered to individual in an effective amount they can activate chemogenetic proteins by direct binding, which will cause a receptor agonism or antagonism which can be identified by a skilled person upon review of the present disclosure in view of the specific actuator and chemogenetic protein and brain regions to be targeted.

For example Clozapine can be administered in doses up to 0.5 mg/kg to activate DREADD receptors, Clozapine-N-Oxide can be administered in doses up to 20 mg/kg for DREADD receptors; Compound 21 can be administered in doses up to 10 mg/kg, doxycycline can be administered in doses up to 20 mg/kg in research species, and up to 4.4 mg/kg in humans and non-human primates, Ivermectin can be administered in doses up to 400 micrograms/per kg to activate GluCl chemogenetic receptor) in humans and non-human primates; 200 micrograms to 10 mg per kg in mice and rats with a preferred dose of 500 mcg/kg).

The chemical actuator can be administrated to an individual through various administration routes including oral ingestion, intravenous, intraperitoneal, or subcutaneous injections, inhalation, intranasal application and others as will be recognized by a person skilled in the art. In some embodiments, the chemical actuator can be administered by intravenous administration The chemical actuators can be in a form of an aqueous solution, solid powder, tablets, aerosols or other forms as will be understood by a person skilled in the art.

In some embodiments, administration of the chemical actuator is performed to chemogenetically treated target brain regions which are target brain regions where the AAV vector of the disclosure has been delivered and the chemogenetic protein has been expressed.

Typical timing between applying focused ultrasound in combination with the administering the contrast agent and administering the AAV vector of the disclosure, and expression of the chemogenetic protein in the target brain region is at least 1 week or later, to ensure BBB closure and allow gene expression in the brain cells.

In an exemplary embodiment, administration of the chemical actuator is first performed 6 to 8 weeks after the acoustically delivering herein described. Proof of concept shows efficacy at 6 to 22 weeks after acoustic delivery.

Conversion of the chemogenetic proteins from inactive to active, or from active to inactive state can be evaluated by measuring their levels of activity appropriate for the given receptors (e.g. histological levels of nuclear c-Fos protein for GPCR activation) or changes in membrane potentials through patch-clamping or electrophysiological recording in the brain for ion channels.

In some embodiments of the methods and systems herein described, the focused ultrasound is applied with single and/or multi-element ultrasound transducers operating at frequencies between 0.2 and 10 MHz, and particularly between 1 and 10 MHz for rats and mice, and 0.2 to 1.5 MHz for non-human primates and humans. The mechanical index is maintained between 0.2 and 1.9, preferably between 0.2 and 0.6. Microbubbles are systematically administered prior to the application of the focused ultrasound at a concentration in the range of and 1.2E7-1E10 per kg of body weight of the individual. The methods further comprise before or after applying focused ultrasound, systematically administering a viral vector encoding a chemogenetic protein gene, preferably AAV and variants thereof, in a concentration between 1E9 and 1E12 viral particle per gram of body weight of the individual. The chemogenetic proteins can be kinases, non-kinase enzymes, G protein-coupled receptors (GPCRs). ligand-gated ion channels or transcription factors that can be recognized by a corresponding chemical actuator, which upon binding to the chemogenetic protein triggers the activation or inhibition of the targeted brain cells. The chemical actuator can be administered at least one week after the administration of the viral vectors. The methods and systems described herein can achieve in some embodiments the controlled percentage population of at least 40% and preferably at least 50% in the target brain region.

In some embodiments of the methods and systems of the disclosure chemogenetic protein and a corresponding chemical actuator or metabolite thereof are selected to activate or inhibit the activity of a target brain cell associated with the target behavior or physiological function of an individual.

In some embodiments, the applying, the systemically administering an effective amount of a microbubble contrast agent and the systemically administering an effective amount of an AAV vector are performed to deliver and express the gene encoding a chemogenetic protein in a controlled percentage population of the target brain cell in the target brain region associate with the target behavior or physiological function, and to obtain a chemogenetically treated target brain region in which the controlled percentage population of the target brain cell comprises the chemogenetic protein. In some embodiments, the controlled percentage population is preferably at least 40% more preferably at least 50%. In some embodiments of the methods and systems of the disclosure, chemogenetic protein and a corresponding chemical actuator or metabolite thereof are selected to activate or inhibit the activity of a target brain cell associated with treatment or prevention of a condition in the individual.

Also disclosed herein are pharmaceutical compositions comprising one or more of the rAAV viruses disclosed herein and one or more pharmaceutically acceptable carriers. The compositions can also comprise additional ingredients such as diluents, stabilizers, excipients, and adjuvants. As used herein, “pharmaceutically acceptable” carriers, excipients, diluents, adjuvants, or stabilizers are nontoxic to the cell or subject being exposed thereto (preferably inert) at the dosages and concentrations employed or that have an acceptable level of toxicity as determined by the skilled practitioners. The carriers, diluents and adjuvants can include buffers such as phosphate, citrate, or other organic acids: antioxidants such as ascorbic acid; low molecular weight polypeptides (e.g., less than about 10 residues); proteins such as serum albumin, gelatin or immunoglobulins; hydrophilic polymers such as polyvinylpyrrolidone; amino acids such as glycine, glutamine, asparagine, arginine, or lysine; monosaccharides, di saccharides, and other carbohydrates including glucose, mannose, or dextrins; chelating agents such as EDTA; sugar alcohols such as mannitol or sorbitol; salt-forming counterions such as sodium; and/or nonionic surfactants such as Tween™, Pluronics™ or polyethylene glycol (PEG). In some embodiments, the physiologically acceptable carrier is an aqueous pH buffered solution.

Disclosed herein include methods of delivering an agent to a target brain region(s) of a subject. In some embodiments, the method comprises: providing an AAV vector comprising an AAV capsid protein disclosed herein. In some embodiments, the AAV vector comprises an agent to be delivered to the target brain region(s). In some embodiments, the method comprises administering the AAV vector to the subject. The composition can be for intravenous administration. The composition can be for systemic administration. The agent can be delivered to endothelial lining of the ventricles in the brain, central canal of the spinal cord, capillaries in the brain, arterioles in the brain, arteries in the brain, or a combination thereof of the subject. The subject can be an adult animal.

Titers of the rAAV to be administered will vary depending, for example, on the particular rAAV, the mode of administration, the treatment goal, the individual, and the cell type(s) being targeted, and can be determined by methods standard in the art. As will be readily apparent to one skilled in the art, the useful in vivo dosage of the recombinant virus to be administered and the particular mode of administration will vary depending upon the age, weight, the severity of the affliction, and animal species treated, the particular recombinant virus expressing the protein of interest that is used, and the specific use for which the recombinant virus is employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

The exact dosage can be determined on a drug-by-drug basis, in most cases, some generalizations regarding the dosage can be made. In some embodiments, the rAAV for delivery of an agent to the target brain region(s) of a subject can be administered, for example via injection, to a subject at a dose of between 1×10¹⁰ viral genome (vg) of the recombinant virus per kg of the subject and 2×10¹⁴ vg per kg, for example between 5×10¹¹ vg/kg and 5×10¹² vg/kg. In some embodiments, the dose of the rAAV administered to the subject is no more than 2×10¹⁴ vg per kg. In some embodiments, the dose of the rAAV administered to the subject is no more than 5×10¹² vg per kg, or no more than 5×10¹¹ vg per kg.

An effective dose and dosage of pharmaceutical compositions to prevent or treat the disease or condition disclosed herein is defined by an observed beneficial response related to the disease or condition, or symptom of the disease or condition. Beneficial response comprises preventing, alleviating, arresting, or curing the disease or condition, or symptom of the disease or condition. In some embodiments, the beneficial response may be measured by detecting a measurable improvement in the presence, level, or activity, of biomarkers, transcriptomic risk profile, or intestinal microbiome in the subject. An “improvement,” as used herein refers to shift in the presence, level, or activity towards a presence, level, or activity, observed in normal individuals (e.g. individuals who do not suffer from the disease or condition). In instances wherein the therapeutic rAAV composition is not therapeutically effective or is not providing a sufficient alleviation of the disease or condition, or symptom of the disease or condition, then the dosage amount and/or route of administration may be changed, or an additional agent may be administered to the subject, along with the therapeutic rAAV composition. In some embodiments, as a patient is started on a regimen of a therapeutic rAAV composition, the patient is also weaned off (e.g., step-wise decrease in dose) a second treatment regimen.

In some embodiments, pharmaceutical compositions in accordance with the present disclosure is administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect. It will be understood that the above dosing concentrations may be converted to vg or viral genomes per kg or into total viral genomes administered by one of skill in the art.

In some embodiments, a dose of the pharmaceutical composition comprises a concentration of infectious particles of at least or about 10⁷, 10⁸, 10⁹, 10¹⁰, 10¹¹, 10¹², 10¹³, 10¹⁴, 10¹⁵, 10¹⁶, or 10¹⁷. In some cases the concentration of infectious particles is 2×10⁷, 2×10⁸, 2×10⁹, 2×10¹⁰, 2×10¹¹, 2×10¹², 2×10¹³, 2×10¹⁴, 2×10¹⁵, 2×10¹⁶, 2×10¹⁷, or a range between any two of these values. In some cases the concentration of the infectious particles is 3×10⁷, 3×10⁸, 3×10⁹, 3×10¹⁰, 3×10¹¹, 3×10¹², 3×10¹³, 3×10¹⁴, 3×10¹⁵, 3×10¹⁶, 3×10¹⁷, or a range between any two of these values. In some cases the concentration of the infectious particles is 4×10⁷, 4×10⁸, 4×10⁹, 4×10¹⁰, 4×10¹¹, 4×10¹², 4×10¹³, 4×10¹⁴, 4×10¹⁵, 4×10¹⁶, 4×10¹⁷, or a range between any two of these values. In some cases the concentration of the infectious particles is 5×10⁷, 5×10⁸, 5×10⁹, 5×10¹⁰, 5×10¹¹, 5×10¹², 5×10¹³, 5×10¹⁴, 5×10¹⁵, 5×10¹⁶, 5×10¹⁷, or a range between any two of these values. In some cases the concentration of the infectious particles is 6×10⁷, 6×10⁸, 6×10⁹, 6×10¹⁰, 6×10¹¹, 6×10¹², 6×10¹³, 6×10¹⁴, 6×10¹⁵, 6×10¹⁶, 6×10¹⁷, or a range between any two of these values. In some cases the concentration of the infectious particles is 7×10⁷, 7×10⁸, 7×10⁹, 7×10¹⁰, 7×10¹¹, 7×10¹², 7×10¹³, 7×10¹⁴, 7×10¹⁵, 7×10⁶, 7×10¹⁷, or a range between any two of these values. In some cases the concentration of the infectious particles is 8×10⁷, 8×10⁸, 8×10⁹, 8×10¹⁰, 8×10¹¹, 8×10¹², 8×10¹³, 8×10¹⁴, 8×10¹⁵, 8×10¹⁶, 8×10¹⁷, or a range between any two of these values. In some cases the concentration of the infectious particles is 9×10⁷, 9×10⁸, 9×10⁹, 9×10¹⁰, 9×10¹¹, 9×10¹², 9×10¹³, 9×10¹⁴, 9×10¹⁵, 9×10¹⁶, 9×10¹⁷, or a range between any two of these values.

The recombinant viruses disclosed herein can be administered to a subject (e.g., a human) in need thereof. The route of the administration is not particularly limited. For example, a therapeutically effective amount of the recombinant viruses can be administered to the subject by via routes standard in the art. The administration can be a systemic administration. The administration can be an intravenous administration.

Non-limiting examples of the route include intramuscular, intravaginal, intravenous, intraperitoneal, subcutaneous, epicutaneous, intradermal, rectal, intraocular, pulmonary, intracranial, intraosseous, oral, buccal, systematic, or nasal. In some embodiments, the recombinant virus is administered to the subject by systematic transduction. In some embodiments, the recombinant virus is administered to the subject by intramuscular injection. In some embodiments, the rAAV is administered to the subject by the parenteral route (e.g., by intravenous, intramuscular or subcutaneous injection), by surface scarification or by inoculation into a body cavity of the subject. Route(s) of administration and serotype(s) of AAV components of the rAAV virus can be readily determined by one skilled in the art taking into account the infection and/or disease state being treated and the target cells/tissue(s) that are to express the protein of interest. In some embodiments, it can be advantageous to administer the rAAV via intravenous administration. The variant AAV provided herein can advantageously provide for intravenous administration of vectors with enhanced tropisms for target brain region(s).

The variant AAV capsid can comprise tropism for a target tissue or a target cell. The target tissue or the target cell can comprise a tissue or a cell of a target brain region(s). The target cell can be a neuronal cell, a neural stem cell, an astrocytes, or a tumor cell. The target cell can be located in a brain or spinal cord. The target cell can comprise an antigen-presenting cell, a dendritic cell, a macrophage, a neural cell, a brain cell, an astrocyte, a microglial cell, and a neuron. In some embodiments, the target cell is an endothelial cell.

In some embodiments, the target cell is a hematopoietic cell, Purkinje cell, Schwann cell, glial cell, astroblast, astrocyte, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, foam cell, histiocyte, luteal cell, lymphocytic stem cell, lymphoid cell, lymphoid stem cell, macroglial cell, mast cell, medulloblast, megakaryoblast, megakaryocyte, metamyelocyte, monoblast, monocyte, myoblast, myocyte, muscle cell, smooth muscle cell, myelocyte, myeloid cell, myeloid stem cell, myoblast, myoepithelial cell, myofibrobast, neuroblast, neuroepithelial cell, neuron, pericyte, peripheral blood mononuclear cell, pinealocyte, pituicyte, plasma cell, platelet, reticulocyte, somatotroph, stem cell, teloglial cell, a zymogenic cell, or any combination thereof. The stem cell can comprise an embryonic stem cell, an induced pluripotent stem cell (iPSC), a hematopoietic stem/progenitor cell (HSPC), or any combination thereof.

Actual administration of the rAAV can be accomplished by using any physical method that will transport the rAAV into the target tissue of the subject. For example, the rAAV disclosed herein can advantageously be administered intravenously for delivery to the target brain region(s). As disclosed herein, capsid proteins of the rAAV can be modified so that the rAAV is targeted to a particular target environment of interest such as target brain region(s), and to enhance tropism to the target environment of interest. Pharmaceutical compositions can be prepared, for example, as injectable formulations.

The AAV can be administered at a dose at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) lower as compared to a comparable method wherein the corresponding parental AAV that does not comprise the acoustic targeting peptide is administered. The FUS can be administered at a dose at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) lower as compared to a comparable method wherein the corresponding parental AAV that does not comprise the acoustic targeting peptide is administered. In some embodiments, the AAV demonstrates at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) reduced transduction of cells of peripheral tissue(s) and/or an at least about 1.1-fold greater transduction of target brain region(s) as compared to the corresponding parental AAV that does not comprise the acoustic targeting peptide.

The AAV can have an at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) improvement in targeting efficiency as compared to the corresponding parental AAV that does not comprise the acoustic targeting peptide, wherein targeting efficiency is the ratio of the brain transduction efficiency to liver transduction efficiency relative to the corresponding parental AAV that does not comprise the acoustic targeting peptide. In some embodiments, the AAV demonstrate an at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) increase in target brain cell(s) transduced per virus administered and/or an at least about 1.1-fold decrease in peripheral transduction per virus administered as compared to the corresponding parental AAV that does not comprise the acoustic targeting peptide. In some embodiments, the AAV demonstrates an at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) greater transduction of one or more target brain region(s) (e.g., the cortex, striatum, thalamus, hippocampus, and/or midbrain) as compared to the corresponding parental AAV that does not comprise the acoustic targeting peptide.

The administering can comprise systemic administration. The systemic administration can be intravenous, intramuscular, intraperitoneal, or intraarticular. Administering can comprise intrathecal administration, intracranial injection, aerosol delivery, nasal delivery, vaginal delivery, direct injection to any tissue in the body, intraventricular delivery, intraocular delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof.

The target brain region(s) can have a size in a range between 1 and 10 mm. The target brain region(s) can comprise the Lateral parabrachial nucleus, brainstem, Medulla oblongata, Medullary pyramids, Olivary body, Inferior olivary nucleus, Rostral ventrolateral medulla, Respiratory center, Dorsal respiratory group, Ventral respiratory group, Pre-Botzinger complex, Botzinger complex, Paramedian reticular nucleus, Cuneate nucleus, Gracile nucleus, Intercalated nucleus, Area postrema, Medullary cranial nerve nuclei, Inferior salivatory nucleus, Nucleus ambiguus, Dorsal nucleus of vagus nerve, Hypoglossal nucleus, Solitary nucleus, Pons, Pontine nuclei, Pontine cranial nerve nuclei, chief or pontine nucleus of the trigeminal nerve sensory nucleus (V), Motor nucleus for the trigeminal nerve (V), Abducens nucleus (VI), Facial nerve nucleus (VII), vestibulocochlear nuclei (vestibular nuclei and cochlear nuclei) (VIII), Superior salivatory nucleus, Pontine tegmentum, Respiratory centers, Pneumotaxic center, Apneustic center, Pontine micturition center (Barrington's nucleus), Locus coeruleus, Pedunculopontine nucleus, Laterodorsal tegmental nucleus, Tegmental pontine reticular nucleus, Superior olivary complex, Paramedian pontine reticular formation, Cerebellar peduncles, Superior cerebellar peduncle, Middle cerebellar peduncle, Inferior cerebellar peduncle, Cerebellum, Cerebellar vermis, Cerebellar hemispheres, Anterior lobe, Posterior lobe, Flocculonodular lobe, Cerebellar nuclei, Fastigial nucleus, Interposed nucleus, Globose nucleus, Emboliform nucleus, Dentate nucleus, Tectum, Corpora quadrigemina, inferior colliculi, superior colliculi, Pretectum, Tegmentum, Periaqueductal gray, Parabrachial area, Medial parabrachial nucleus, Subparabrachial nucleus (Kölliker-Fuse nucleus), Rostral interstitial nucleus of medial longitudinal fasciculus, Midbrain reticular formation, Dorsal raphe nucleus, Red nucleus, Ventral tegmental area, Substantia nigra, Pars compacta, Pars reticulata, Interpeduncular nucleus, Cerebral peduncle, Crus cerebri, Mesencephalic cranial nerve nuclei, Oculomotor nucleus (III), Trochlear nucleus (IV), Mesencephalic duct (cerebral aqueduct, aqueduct of Sylvius), Pineal body, Habenular nucleim Stria medullares, Taenia thalami, Subcommissural organ, Thalamus, Anterior nuclear group, Anteroventral nucleus (aka ventral anterior nucleus), Anterodorsal nucleus, Anteromedial nucleus, Medial nuclear group, Medial dorsal nucleus, Midline nuclear group, Paratenial nucleus, Reuniens nucleus, Rhomboidal nucleus, Intralaminar nuclear group, Centromedial nucleus, Parafascicular nucleus, Paracentral nucleus, Central lateral nucleus, Central medial nucleus, Lateral nuclear group, Lateral dorsal nucleus, Lateral posterior nucleus, Pulvinar, Ventral nuclear group, Ventral anterior nucleus, Ventral lateral nucleus, Ventral posterior nucleus, Ventral posterior lateral nucleus, Ventral posterior medial nucleus, Metathalamus, Medial geniculate body, Lateral geniculate body, Thalamic reticular nucleus, Hypothalamus, limbic system, HPA axis, preoptic area, Medial preoptic nucleus, Suprachiasmatic nucleus, Paraventricular nucleus, Supraoptic nucleusm Anterior hypothalamic nucleus, Lateral preoptic nucleus, median preoptic nucleus, periventricular preoptic nucleus, Tuberal, Dorsomedial hypothalamic nucleus, Ventromedial nucleus, Arcuate nucleus, Lateral area, Tuberal part of Lateral nucleus, Lateral tuberal nuclei, Mammillary nuclei, Posterior nucleus, Lateral area, Optic chiasm, Subfornical organ, Periventricular nucleus, Pituitary stalk, Tuber cinereum, Tuberal nucleus, Tuberomammillary nucleus, Tuberal region, Mammillary bodies, Mammillary nucleus, Subthalamus, Subthalamic nucleus, Zona incerta, Pituitary gland, neurohypophysis, Pars intermedia, adenohypophysis, cerebral hemispheres, Corona radiata, Internal capsule, External capsule, Extreme capsule, Arcuate fasciculus, Uncinate fasciculus, Perforant Path, Hippocampus, Dentate gyms, Cornu ammonis, Cornu ammonis area 1, Cornu ammonis area 2, Cornu ammonis area 3, Cornu ammonis area 4, Amygdala, Central nucleus, Medial nucleus (accessory olfactory system), Cortical and basomedial nuclei, Lateral and basolateral nuclei, extended amygdala, Stria terminalis, Bed nucleus of the stria terminalis, Claustrum, Basal ganglia, Striatum, Dorsal striatum (aka neostriatum), Putamen, Caudate nucleus, Ventral striatum, Striatum, Nucleus accumbens, Olfactory tubercle, Globus pallidus, Subthalamic nucleus, Basal forebrain, Anterior perforated substance, Substantia innominata, Nucleus basalis, Diagonal band of Broca, Septal nuclei, Medial septal nuclei, Lamina terminalis, Vascular organ of lamina terminalis, Olfactory bulb, Piriform cortex, Anterior olfactory nucleus, Olfactory tract, Anterior commissure, Uncus, Cerebral cortex, Frontal lobe, Frontal cortex, Primary motor cortex, Supplementary motor cortex, Premotor cortex, Prefrontal cortex, frontopolar cortex, Orbitofrontal cortex, Dorsolateral prefrontal cortex, dorsomedial prefrontal cortex, ventrolateral prefrontal cortex, Superior frontal gyms, Middle frontal gyms, Inferior frontal gyms, Brodmann areas (4, 6, 8, 9, 10, 11, 12, 24, 25, 32, 33, 44, 45, 46, and/or 47), Parietal lobe, Parietal cortex, Primary somatosensory cortex (51), Secondary somatosensory cortex (S2), Posterior parietal cortex, postcentral gyms, precuneus, Brodmann areas (1, 2, 3 (Primary somesthetic area), 5, 7, 23, 26, 29, 31, 39, and/or 40), Occipital lobe, Primary visual cortex (V1), V2, V3, V4, V5/MT, Lateral occipital gyms, Cuneus, Brodmann areas (17 (V1, primary visual cortex), 18, and/or 19), temporal lobe, Primary auditory cortex (A1), secondary auditory cortex (A2), Inferior temporal cortex, Posterior inferior temporal cortex, Superior temporal gyms, Middle temporal gyms, Inferior temporal gyms, Entorhinal Cortex, Perirhinal Cortex, Parahippocampal gyms, Fusiform gyms, Brodmann areas (9, 20, 21, 22, 27, 34, 35, 36, 37, 38, 41, and/or 42), Medial superior temporal area (MST), insular cortex, cingulate cortex, Anterior cingulate, Posterior cingulate, dorsal cingulate, Retrosplenial cortex, Indusium griseum, Subgenual area 25, Brodmann areas (23, 24; 26, 29, 30 (retrosplenial areas), 31, and/or 32), cranial nerves (Olfactory (I), Optic (II), Oculomotor (III), Trochlear (IV), Trigeminal (V), Abducens (VI), Facial (VII), Vestibulocochlear (VIII), Glossopharyngeal (IX), Vagus (X), Accessory (XI), Hypoglossal (XII)), or any combination thereof.

The brain region can comprise neural pathways Superior longitudinal fasciculus, Arcuate fasciculus, Thalamocortical radiations, Cerebral peduncle, Corpus callosum, Posterior commissure, Pyramidal or corticospinal tract, Medial longitudinal fasciculus, dopamine system, Mesocortical pathway, Mesolimbic pathway, Nigrostriatal pathway, Tuberoinfundibular pathway, serotonin system, Norepinephrine Pathways, Posterior column-medial lemniscus pathway, Spinothalamic tract, Lateral spinothalamic tract, Anterior spinothalamic tract, or any combination thereof.

The recombinant virus to be used can be utilized in liquid or freeze-dried form (in combination with one or more suitable preservatives and/or protective agents to protect the virus during the freeze-drying process). For gene therapy (e.g., of neurological disorders which may be ameliorated by a specific gene product) a therapeutically effective dose of the recombinant virus expressing the therapeutic protein is administered to a host in need of such treatment. The use of the recombinant virus disclosed herein in the manufacture of a medicament for inducing immunity in, or providing gene therapy to, a host is within the scope of the present application.

In instances where human dosages for the rAAV have been established for at least some condition, those same dosages, or dosages that are between about 0.1% and 500%, more preferably between about 25% and 250% of the established human dosage can be used. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

A therapeutically effective amount of the rAAV can be administered to a subject at various points of time. For example, the rAAV can be administered to the subject prior to, during, or after the subject has developed a disease or disorder. The rAAV can also be administered to the subject prior to, during, or after the occurrence of a disease or disorder (e.g., Huntington's disease (HD), Alzheimer's disease, Parkinson's disease, Amyotrophic lateral sclerosis, spinal muscular atrophy, types I and II, Friedreich's Ataxia, Spinocerebellar ataxia and any of the lysosomal storage disorders that involve cells with target brain region(s), which includes but is not limited to Krabbe disease, Sandhoff disease, Tay-Sachs, Gaucher disease (Type I, II, or 111), Niemann-Pick disease (NPC1 or NPC2 deficiency), Hurler syndrome, Pompe disease, Batten disease, or any combination thereof), chronic pain, or a combination thereof. In some embodiments, the rAAV is administered to the subject during remission of the disease or disorder. In some embodiments, the rAAV is administered prior to the onset of the disease or disorder in the subject. In some embodiments, the rAAV is administered to a subject at a risk of developing the disease or disorder.

The disease or disorder can comprise a neurological disease or disorder. For example, the neurological disease or disorder can comprise epilepsy, Dravet Syndrome, Lennox Gastaut Syndrome, myocolonic seizures, juvenile myocolonic epilepsy, refractory epilepsy, schizophrenia, juvenile spasms, West syndrome, infantile spasms, refractory infantile spasms, Alzheimer's disease, Creutzfeld-Jakob's syndrome/disease, bovine spongiform encephalopathy (BSE), prion related infections, diseases involving mitochondrial dysfunction, diseases involving β-amyloid and/or tauopathy, Down's syndrome, hepatic encephalopathy, Huntington's disease, motor neuron diseases, amyotrophic lateral sclerosis (ALS), olivoponto-cerebellar atrophy, post-operative cognitive deficit (POCD), systemic lupus erythematosus, systemic sclerosis, Sjogren's syndrome, Neuronal Ceroid Lipofuscinosis, neurodegenerative cerebellar ataxias, Parkinson's disease, Parkinson's dementia, mild cognitive impairment, cognitive deficits in various forms of mild cognitive impairment, cognitive deficits in various forms of dementia, dementia pugilistica, vascular and frontal lobe dementia, cognitive impairment, learning impairment, eye injuries, eye diseases, eye disorders, glaucoma, retinopathy, macular degeneration, head or brain or spinal cord injuries, head or brain or spinal cord trauma, convulsions, epileptic convulsions, epilepsy, temporal lobe epilepsy, myoclonic epilepsy, tinnitus, dyskinesias, chorea, Huntington's chorea, athetosis, dystonia, stereotypy, ballism, tardive dyskinesias, tic disorder, torticollis spasmodicus, blepharospasm, focal and generalized dystonia, nystagmus, hereditary cerebellar ataxias, corticobasal degeneration, tremor, essential tremor, addiction, anxiety disorders, panic disorders, social anxiety disorder (SAD), attention deficit hyperactivity disorder (ADHD), attention deficit syndrome (ADS), restless leg syndrome (RLS), hyperactivity in children, autism, dementia, dementia in Alzheimer's disease, dementia in Korsakoff syndrome, Korsakoff syndrome, vascular dementia, dementia related to HIV infections, HIV-1 encephalopathy, AIDS encephalopathy, AIDS dementia complex, AIDS-related dementia, major depressive disorder, major depression, depression, memory loss, stress, bipolar manic-depressive disorder, drug tolerance, drug tolerance to opioids, movement disorders, fragile-X syndrome, irritable bowel syndrome (IBS), migraine, multiple sclerosis (MS), muscle spasms, pain, chronic pain, acute pain, inflammatory pain, neuropathic pain, posttraumatic stress disorder (PTSD), schizophrenia, spasticity, Tourette's syndrome, eating disorders, food addiction, binge eating disorders, agoraphobia, generalized anxiety disorder, obsessive-compulsive disorder, panic disorder, social phobia, phobic disorders, substance-induced anxiety disorder, delusional disorder, schizoaffective disorder, schizophreniform disorder, substance-induced psychotic disorder, hypertension, or any combination thereof.

Disclosed herein, in some embodiments, are formulations of pharmaceutically-acceptable excipients and carrier solutions suitable for delivery of the rAAV compositions described herein, as well as suitable dosing and treatment regimens for using the particular compositions described herein in a variety of treatment regimens. In some embodiments, the amount of therapeutic gene expression product in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable. In some instances, the rAAV compositions are suitably formulated pharmaceutical compositions disclosed herein, to be delivered either intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs by direct injection.

In some embodiments, the pharmaceutical forms of the AAV-based viral compositions suitable for injectable use include sterile aqueous solutions or dispersions and sterile powders for the extemporaneous preparation of sterile injectable solutions or dispersions. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In some embodiments, for administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Disclosed herein are sterile injectable solutions comprising the rAAV compositions disclosed herein, which are prepared by incorporating the rAAV compositions disclosed herein in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Injectable solutions may be advantageous for systemic administration, for example by intravenous administration.

Also provided herein are formulations in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

Formulations for intranasal administration can comprise a coarse powder comprising the active ingredient and having an average particle size from about 0.2 μm to 500 μm. Such formulations are administered in the manner in which snuff is taken, e.g. by rapid inhalation through the nasal passage from a container of the powder held close to the nose. Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, comprise 0.1% to 20% (w/w) active ingredient, the balance comprising an orally dissolvable and/or degradable composition and, optionally, one or more of the additional ingredients described herein. Alternately, formulations suitable for buccal administration may comprise powders and/or an aerosolized and/or atomized solutions and/or suspensions comprising active ingredients. Such powdered, aerosolized, and/or aerosolized formulations, when dispersed, may comprise average particle and/or droplet sizes in the range of from about 0.1 nm to about 200 nm, and may further comprise one or more of any additional ingredients described herein.

Suitable dose and dosage administrated to a subject is determined by factors including, but not limited to, the particular therapeutic rAAV composition, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject or host being treated.

The amount of AAV compositions and time of administration of such compositions will be within the purview of the skilled artisan having benefit of the present teachings. In some embodiments, however, that the administration of therapeutically-effective amounts of the disclosed compositions may be achieved by a single administration, such as for example, a single injection of sufficient numbers of infectious particles to provide therapeutic benefit to the patient undergoing such treatment. This is made possible, at least in part, by the fact that certain target cells (e.g., neurons) do not divide, obviating the need for multiple or chronic dosing.

In some embodiments, it is advantageous to provide multiple, or successive administrations of the AAV vector compositions, either over a relatively short, or a relatively prolonged period of time, as may be determined by the medical practitioner overseeing the administration of such compositions. For example, the number of infectious particles administered to a mammal may be on the order of about 10⁷, 10⁸, 10⁹, 10¹⁰, 10¹¹, 10¹², 10¹³, or even higher, infectious particles/ml given either as a single dose, or divided into two or more administrations as may be required to achieve therapy of the particular disease or disorder being treated. In fact, in certain embodiments, it may be desirable to administer two or more different AAV vector compositions, either alone, or in combination with one or more other therapeutic drugs to achieve the desired effects of a particular therapy regimen. In various embodiments, the daily and unit dosages are altered depending on a number of variables including, but not limited to, the activity of the therapeutic rAAV composition used, the disease or condition to be treated, the mode of administration, the requirements of the individual subject, the severity of the disease or condition being treated, and the judgment of the practitioner.

The dosing frequency of the rAAV virus can vary. For example, the rAAV virus can be administered to the subject about once every week, about once every two weeks, about once every month, about one every six months, about once every year, about once every two years, about once every three years, about once every four years, about once every five years, about once every six years, about once every seven years, about once every eight years, about once every nine years, about once every ten years, or about once every fifteen years. In some embodiments, the rAAV virus is administered to the subject at most about once every week, at most about once every two weeks, at most about once every month, at most about one every six months, at most about once every year, at most about once every two years, at most about once every three years, at most about once every four years, at most about once every five years, at most about once every six years, at most about once every seven years, at most about once every eight years, at most about once every nine years, at most about once every ten years, or at most about once every fifteen years.

Disclosed herein are kits comprising compositions disclosed herein. Also disclosed herein are kits for the treatment or prevention of a disease or disorder described herein. In some instances, the disease or condition is cancer, a pathogen infection, neurological disease, muscular disease, or an immune disorder, such as those described herein. In one embodiment, a kit can include a therapeutic or prophylactic composition containing an effective amount of a composition of a rAAV particle encapsidating a heterologous nucleic acid provided herein and a rAAV capsid protein of the present disclosure. In another embodiment, a kit can include a therapeutic or prophylactic composition containing an effective amount of cells modified by the rAAV described herein (“modified cell”), in unit dosage form that express therapeutic nucleic acid. In some embodiments, a kit comprises a sterile container which can contain a therapeutic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In some embodiments, rAAV are provided together with instructions for administering the rAAV to a subject having or at risk of developing the disease or condition. Instructions can generally include information about the use of the composition for the treatment or prevention of the disease or condition.

In some embodiments, the instructions include at least one of the following: description of the therapeutic rAAV composition; dosage schedule and administration for treatment or prevention of the disease or condition disclosed herein; precautions; warnings; indications; counter-indications; overdosage information; adverse reactions; animal pharmacology; clinical studies; and/or references. The instructions can be printed directly on the container (when present), or as a label applied to the container, or as a separate sheet, pamphlet, card, or folder supplied in or with the container. In some embodiments, instructions provide procedures for administering the rAAV to the subject alone. In some embodiments, instructions provide procedures for administering the rAAV to the subject at least about 1 hour (hr), 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, 25 hrs, 26 hrs, 27 hrs, 28 hrs, 29 hrs, 30 hrs, or up to 2 days, 3 days, 4 days, 5 days, 6 days, or 7 days after or before administering an additional therapeutic agent disclosed herein. In some instances, the instructions provide that the rAAV is formulated for intravenous injection. In some instances, the instructions provide that the rAAV is formulated for intranasal administration.

In some embodiments, the AAV vectors and contrast agent are comprised in a composition together with a compatible vehicle. The term “vehicle” as used herein indicates any of various media acting usually as solvents, carriers, binders or diluents for the AAV vectors, genes, contrast agent and/or chemical actuators herein described that are comprised in the composition as an active ingredient. In some embodiments, the composition including the AAV vectors, genes, contrast agent and/or chemical actuators can be used in one of the methods or systems herein described.

The pharmaceutical preparations of an AAV vector and chemical actuator can be given by forms suitable for each administration route. In some embodiments, the AAV vectors, genes, contrast agent and/or chemical actuators can be provided as part of a system to control neural circuits is described. The system can comprise AAV vectors encoding a chemogenetic receptor gene, one or more chemical actuators, and optionally a contrast agent for simultaneous, combined or sequential use in the method to noninvasively control neural cell activities.

A skilled person will be able to identify suitable combination of AAV vector concentrations and ultrasound frequency taking into account vascularization of the target region, viral tropism and transfer speed of a vector from the blood brain barrier to the target brain region, all factors that may require higher doses as will be understood by a skilled person.

### EXAMPLES

Some aspects of the embodiments discussed above are disclosed in further detail in the following examples, which are not in any way intended to limit the scope of the present disclosure.

### Example 1

### Engineering Viral Vectors for Acoustically Targeted Gene Delivery

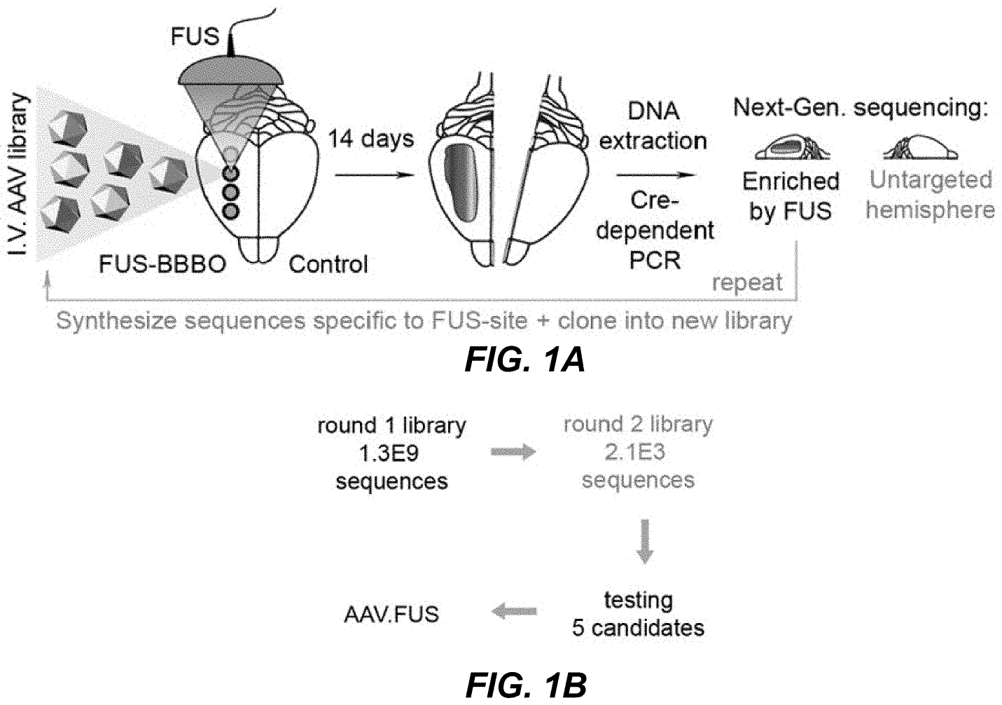

It was reasoned that the limitations of the currently available FUS-BBBO compositions and methods described above arise from the fact that wild-type serotypes of AAV did not evolve to cross physically loosened biophysical barriers and are therefore not optimal for this purpose. It was hypothesized that these limitations could be addressed by developing new engineered viral serotypes specifically optimized for FUS-BBBO delivery. Capsid engineering techniques in which mutations are introduced into viral capsid proteins have been used to enhance gene delivery properties such as tissue specificity, immune evasion, or axonal tracing. However, they have not yet been used to optimize viral vectors to work in conjunction with specific physical delivery mechanisms. To test the hypothesis, in vivo selection of mutagenized AAVs in mice was performed in conjunction with FUS-BBBO (FIG. 1) by adapting a recently developed Cre-recombinase-based screening methodology. 5 viral capsid mutants with enhanced transduction at the site of FUS-BBBO and but not in the untargeted brain regions were identified. Detailed validation experiments were then performed comparing each of these mutants to the parent wild-type AAV, revealing a significant increase in on-target transduction efficiency, increased neuronal tropism, and a marked decrease in off-target transduction in peripheral organs, resulting in an overall performance improvement of more than 10-fold. These results demonstrate the evolvability of AAVs for specific physical delivery methods.

### Results

High-Throughput In Vivo Screening for AAVs with Efficient FUS-BBBO Transduction

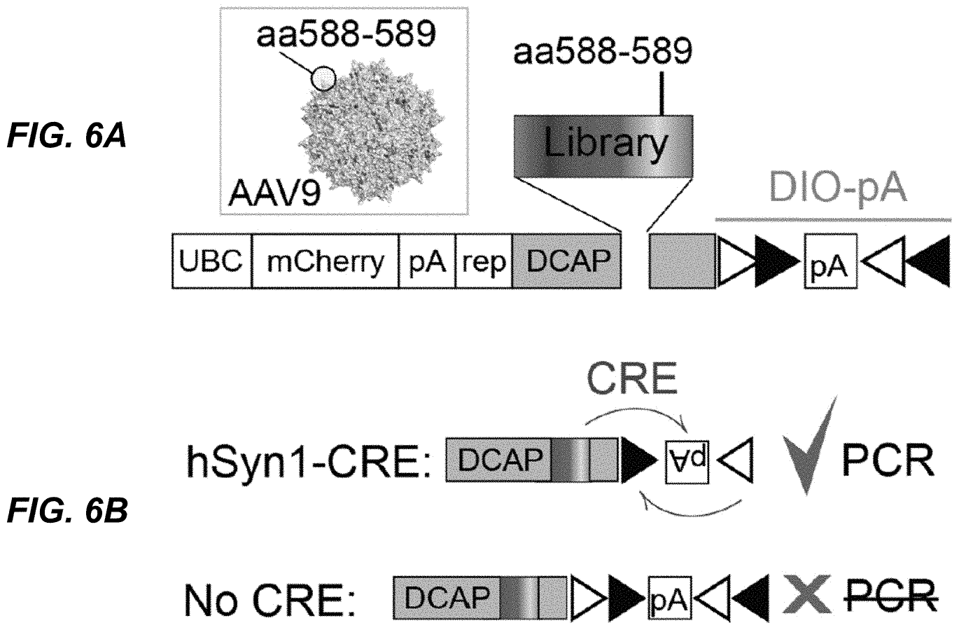

To identify new AAV variants with improved FUS-BBBO-targeted transduction of neurons, a library of viral capsid sequences was generated containing insertions of 7 randomized amino acids between residues 588 and 589 of the AAV9 capsid protein (FIG. 6). AAV9 was chosen as a starting point due to its use in previous FUS-BBBO studies. Meanwhile, 7-mer insertions have been widely used to engineer AAVs with new properties.

To make the screening more efficient, recombination-based AAV selection was employed. In this approach, each viral vector genome in the library encodes a mutated capsid protein alongside another segment of DNA that gets inverted in the presence of Cre recombinase (FIG. 6A). Screening in Cre-expressing cells then allows the identification of capsid variants with the confirmed ability to transduce the cells, with the successful capsid sequences amplified by PCR using inversion-specific primers (FIG. 6B). Using this approach, in vivo selection in mice expressing Cre recombinase under neuron-specific promoters ensures the recovery of neuron-transducing AAV variants.

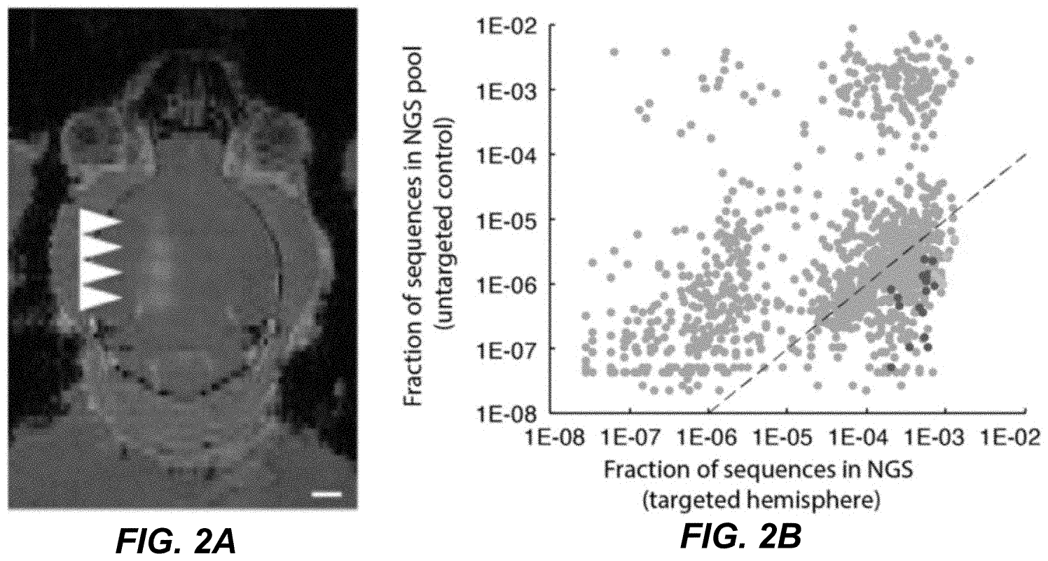

To screen specifically for mutants with enhanced transduction at the site of FUS-BBBO, a two-step strategy was followed (FIGS. 1A-1B). First, the 1.3×10⁹-capsid library was down-selected to a smaller number of variants that were capable of entering the brain at the site of FUS-BBBO, and then re-screened these variants to quantify enhancement and FUS-target specificity of FUS-BBBO-mediated transduction. FUS parameters were employed below tissue damage limits (0.33 MPa at 1.5 MHz, 10 ms pulse length, 1 Hz repetition frequency, 0.22 ml dose of microbubbles per gram of body weight). An AAV library was injected intravenously (IV) and immediately after FUS was applied to the brains of two hSynl-CRE mice to open the BBB, using magnetic resonance imaging (MM) guidance for anatomical targeting, and confirming the opening with contrast-enhanced MM. 4 sites were targeted within one hemisphere (FIG. 2A). The library contained a total of 1.3×10⁹ clones, at a dose of 6.7×10⁹ viral particles per gram of body weight. After 2 weeks the mice were euthanized and their brains were collected. Immediately after, the viral DNA was extracted from the brain and Cre-dependent PCR amplification was used to recover and selectively amplify the viral DNA from AAV particles that transduced neurons. Next-generation sequencing (NGS) was then performed of the capsid region and the 2,098 most abundant sequences were selected for subsequent evaluation. Based on the design of this screen, each of these variants was pre-selected to able to enter the brain at the site of FUS-BBBO and transduce neurons. However, the specificity of FUS-mediated entry and efficiency of transduction by these variants could not be determined quantitatively at this stage due to the small copy number of each variant in the library.

To quantitatively compare the 2,098 down-selected capsid variants, they were re-synthesized and packaged as a new AAV library at a dose of 1.3×10⁹ viral particles per gram of body weight, corresponding to ^{˜}1.5-3×10⁷ viral particles of each clone being injected into each mouse. In each of two hSyn-CRE mice, an AAV library was injected intravenously and the BBB in one hemisphere was opened using MM-guided FUS as in round 1. After 2 weeks, the brains were extracted, hemispheres separated and the DNA from FUS-targeted and untargeted hemispheres extracted for each mouse. The DNA extract was amplified by the CRE-dependent PCR to enrich for viral genomes that transduced neurons. After FUS-BBBO delivery, DNA extraction, CRE-dependent PCR, and NGS, 1,433 sequences were recovered.

To identify the most improved candidates their copy number were examined in the targeted hemispheres and it was compared to the untargeted hemispheres (FIG. 2B). Variants were first looked for that were at least 100-fold more represented in the targeted hemisphere relative to the untargeted hemisphere to identify AAVs that selectively transduced sites subjected to FUS-BBBO. From this list, candidates were further selected for which the 100-fold difference was maintained in both mice and in each alternative codon sequence corresponding to its 7-mer peptide, thus ensuring robustness of the NGS data. In the end, 35 sequences met these criteria (dark grey symbols in FIG. 2B). Among these FUS-BBBO-specific variants, the 5 most frequently detected sequences were chosen, which were hypothesized would code for AAV capsids with the most efficient neuronal transduction. These sequences were re-synthesized, cloned into the AAV9 capsid between amino acids 587-588, and packaged for detailed evaluation, naming them AAV.FUS 1 through 5.

AAV.FUS Candidates Show Enhanced Transduction of Neurons in Targeted Brain Regions and Reduced Transduction of Peripheral Organs

An ideal AAV vector for ultrasound-mediated gene delivery to the brain would efficiently transduce targeted neurons while avoiding peripheral tissues, such as the typically highly transduced liver. Additionally, such a vector should only transduce the brain at the FUS-targeted sites. Of the natural AAV serotypes, AAV9 is most commonly used in FUS-BBBO because it transduces neurons at the ultrasound target with relatively high specificity compared to untargeted brain regions. However, AAV9 requires high doses to achieve significant gene expression and transduces peripheral tissues in the process, leaving substantial room for improvement. It was decided to test the five selected AAV.FUS vectors against AAV9.

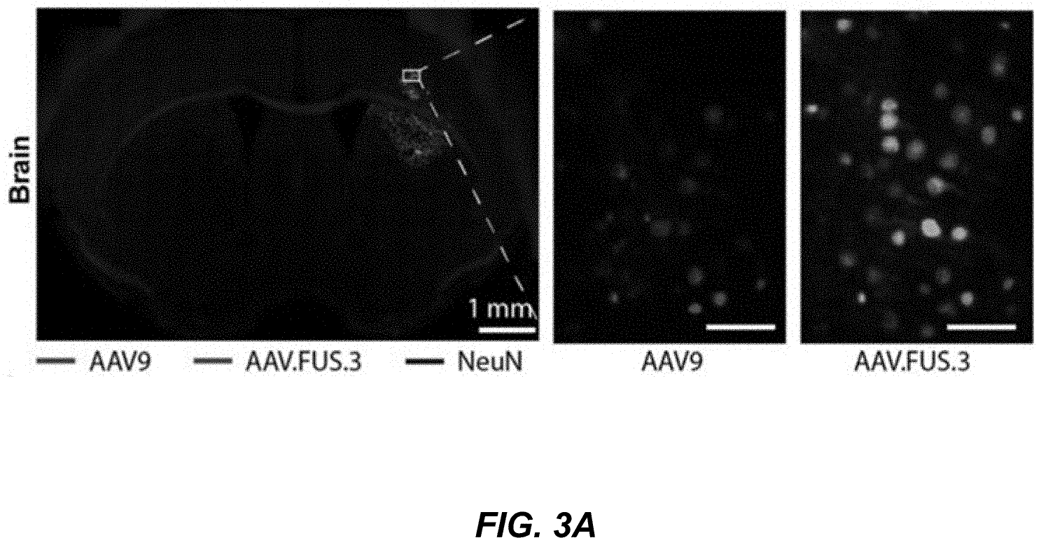

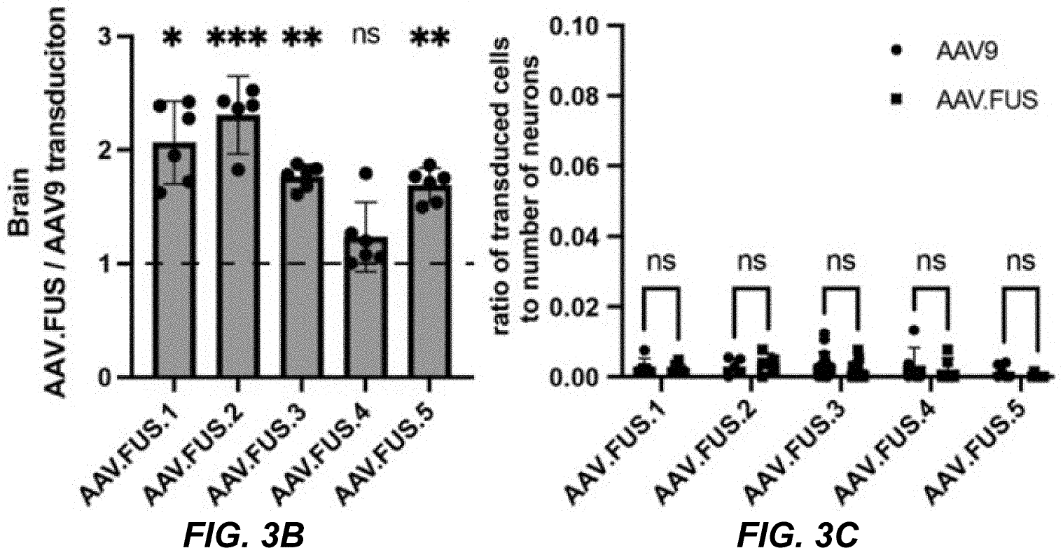

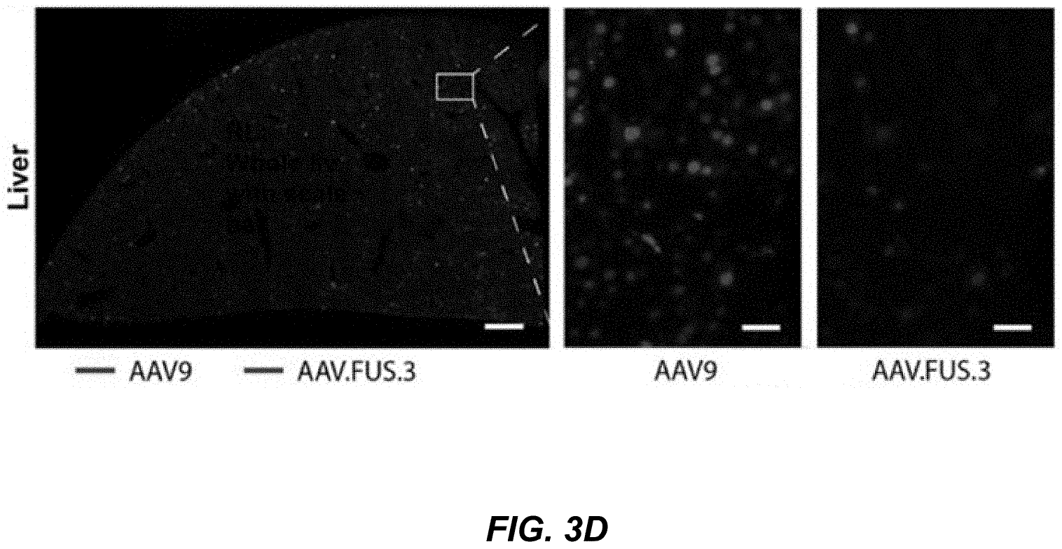

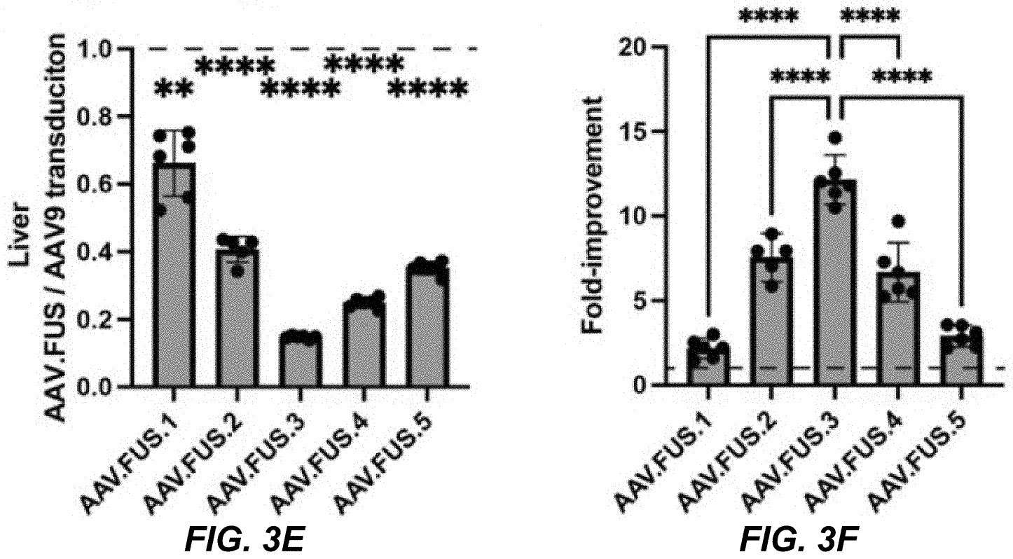

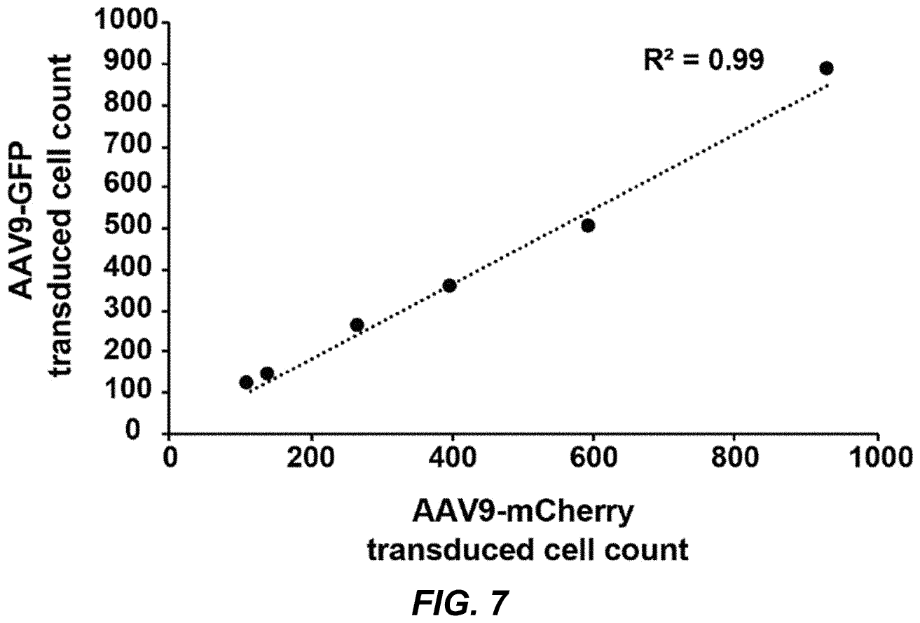

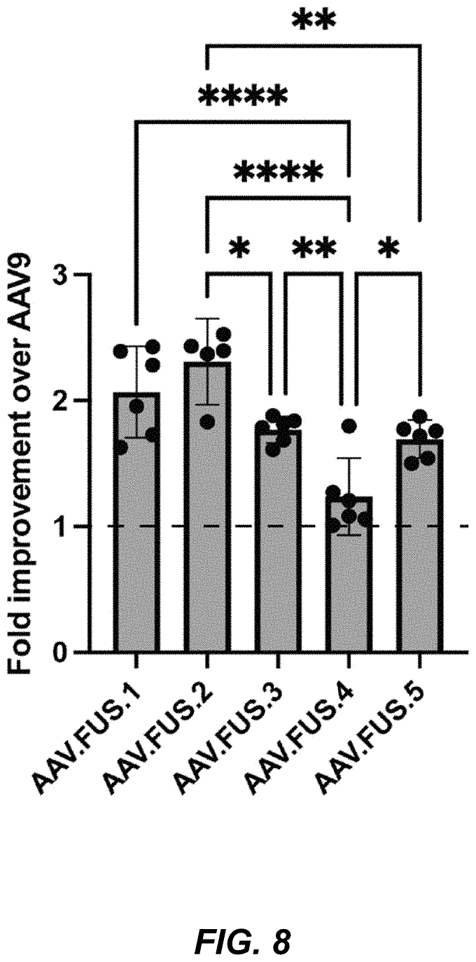

FUS-BBBO was performed while intravenously co-administering each AAV.FUS candidate alongside AAV9 in individual comparison experiments. Consequently, each mouse had an internal control where the location and extent of FUS-BBBO was identical for both serotypes. To quantify the transduction efficiency, the fluorescent proteins mCherry and EGFP were encoded in AAV9 and each AAV.FUS variant, respectively, under a general CaG promoter. After 2 weeks of expression, the numbers of mCherry and EGFP-expressing cells within the site of FUS-BBBO were counted. The reliability of this quantification method was established by comparing cell counts for co-administered AAV9-EGFP and AAV9-mCherry (FIG. 7). The quantification showed that AAV.FUS.1, 2, 3, and 5 had significantly improved transduction efficiency compared to AAV9 (p=0.0274, 0.0003, 0.0052, 0.0087, respectively, FIGS. 3A-3B) whereas AAV.FUS.4 showed no improvement (p=0.2556). The fold-change in transduction relative to AAV9 was greatest for AAV.FUS.2, and lowest for AAV.FUS.4 (FIG. 8). None of the AAV.FUS candidates produced off-target expression within the brain (FIG. 3C).

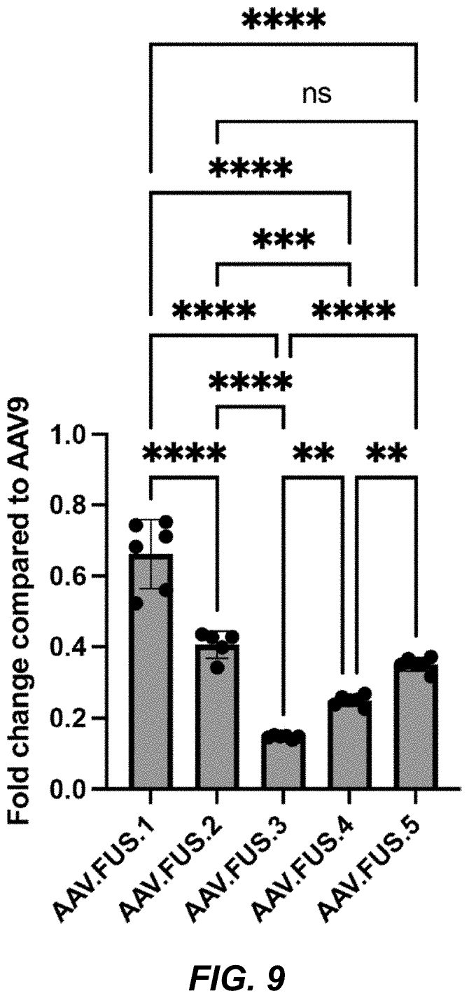

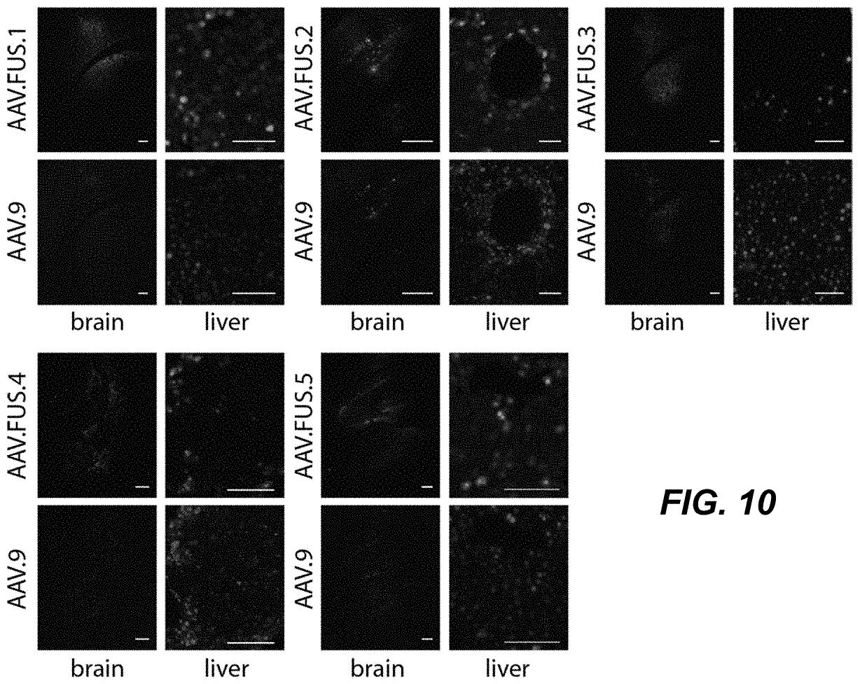

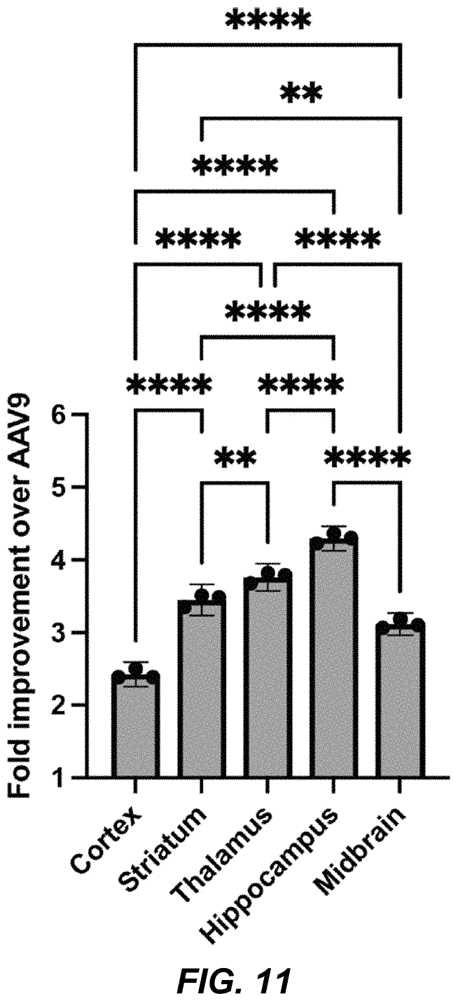

Next, the extent to which AAV.FUS candidates transduce off-target peripheral organs was evaluated. In mice that received intravenous co-injections of AAV9-mCherry and each variant of AAV.FUS-EGFP transduced cells in the liver was counted, a peripheral organ known to be targeted by AAVs and a potential source of dose-limiting toxicity. Two weeks after injection, liver tissues were collected and the livers were imaged, counting cells expressing each fluorophore (FIGS. 3D-3E). It was found that there was markedly reduced liver transduction among the AAV.FUS candidates compared to AAV9 (FIG. 3E). AAV.FUS 3 showed the largest reduction in liver transduction compared to the wild-type serotype (6.8-fold reduction, p<0.0001), which was significantly higher reduction compared to the other tested AAV.FUS candidates (FIG. 9).

The analyses of brain and liver transduction showed that AAV.FUS candidates both decrease the targeting of the peripheral tissue and increase the transduction efficiency of the targeted brain regions, which leads to a large overall improvement in transduction specificity, expressed as the ratio of the fold-increase in brain transduction and the fold-decrease in liver transduction compared to AAV9. By this metric, AAV.FUS.3 showed a 12.1-fold improvement, significantly greater than the other candidates (p<0.0001 for all pairwise comparisons, one-way ANOVA with Tukey-HSD post hoc test; FIG. 3F).

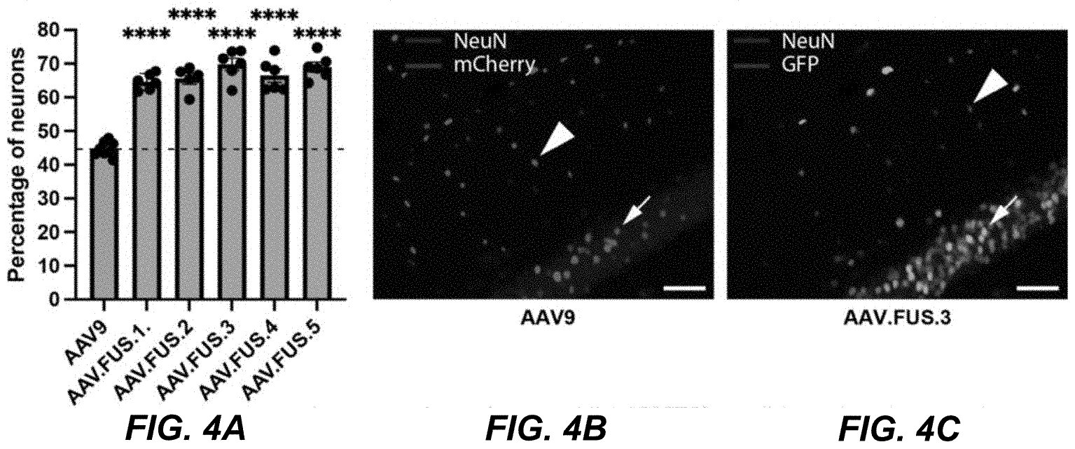

A final criterion for successful gene delivery in many applications is the ability to transduce specific cell-types at the targeted anatomical location, for example neurons. AAV9 transduces both neuronal and non-neuronal cell types. It was hypothesized that, since the Cre-dependent screen used mice with the recombinase expressed under a neuronal promoter, the engineered variants could have a higher neuronal tropism relative to their wild-type parent serotype. To test this hypothesis, brain sections from mice co-transduced with AAV9-mCherry were immunostained and each variant of AAV.FUS-EGFP during FUS-BBBO for the neuronal marker NeuN and these sections were imaged for GFP, mCherry, and NeuN signal. The fraction of AAV9-transduced (mCherry-positive) cells that were also positive for NeuN was 44.7% (±0.75%, SEM; n=8). In contrast, all AAV.FUS candidates had higher neuronal tropism (FIG. 4), with neurons constituting between 64.6 (±0.97%, SEM) (AAV.FUS.1) and 69.8 (±1.8%, SEM) (AAV.FUS.3) of all transduced cells (one-way ANOVA with Tukey-HSD post hoc test; p<0.0001 for all AAV.FUS candidates). These results show that in addition to improved specificity for targeted regions of the brain, the engineered viral capsids are more selective for neurons over other cephalic cell types.

Region-Specific Transduction Efficiency of AAV.FUS.3

Based on its leading combination of neuronal tropism and improvement in brain specificity among the engineered variants, AAV.FUS.3 was selected for further evaluation as a FUS-BBBO-specific viral vector. To further characterize its performance relative to AAV9, it was decided to evaluate the efficiency of delivery when these vectors are targeted to different brain regions. To ensure that each region is targeted exclusively, only one brain region was targeted with FUS-BBBO in each tested mouse. To ensure the rigor of this investigation and account for variability in virus titration, a new batch of both AAV9 and AAV.FUS.3 was obtained and they were tittered independently. The efficiency of transduction was evaluated when these vectors were targeted by FUS-BBBO to the striatum (caudate putamen), thalamus, hippocampus, and midbrain of AAV.FUS.3.

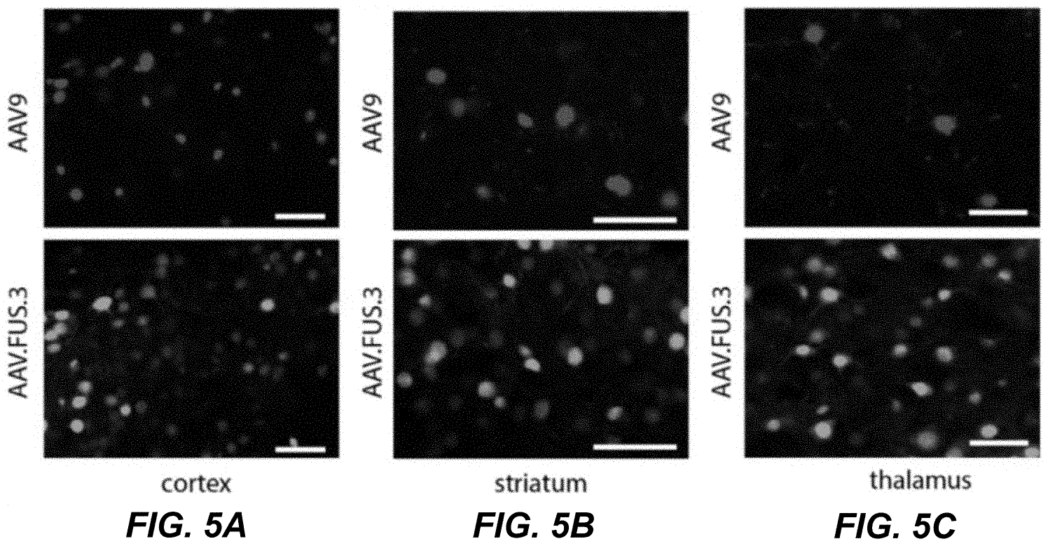

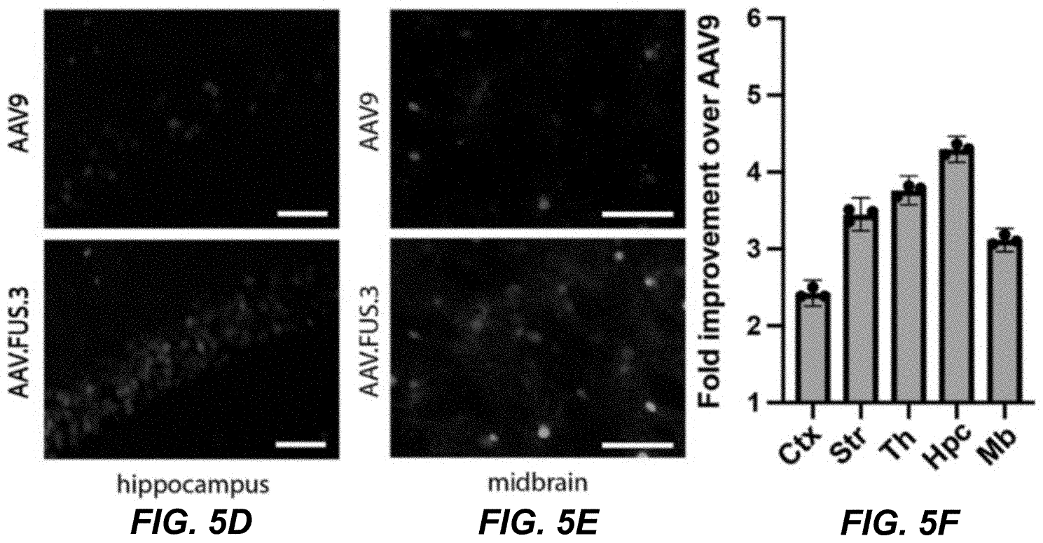

As in the earlier experiments, a major improvement was observed in AAV.FUS.3 transduction compared to AAV9 in all targeted regions, with a fold-change ranging from 2.4±0.08 to 4.3±0.08 (95% CI, FIG. 5). Among brain regions, it was found that the hippocampus (Hpc) is transduced with a particularly elevated relative efficiency, while the cortex (Ctx) showed the lowest, but still substantial, improvement. These results indicate that AAV.FUS.3 can target multiple brain regions with improved efficiency, while suggesting the potential for further engineering AAVs with region-enhanced tropism in FUS-BBBO delivery.

### Discussion

The results show that viral vectors can be engineered to improve noninvasive, site-specific gene delivery to the brain using ultrasound-mediated blood-brain barrier opening. Gene therapy is widely used in research and is becoming a clinical reality. However, most of the available methods for gene delivery to the brain either lack regional specificity or are invasive and challenging to apply to large brain regions. While FUS-BBBO promises to overcome these challenges, its use in conjunction with AAVs encounters challenges of parameter safety and peripheral transduction which, despite longstanding effort, have not been fully solved through the optimization of ultrasound parameters and equipment alone. Simply increasing the intravenous dose of natural AAVs is not feasible due to the additional cost of the virus production, stronger immune response to the virus, higher non-specific transduction of peripheral tissues and associated toxicity.

In this study, the problem of improving FUS-BBBO gene delivery was approached by engineering the viral vectors themselves. The resulting improvements include an increase in brain transduction per virus injected, a reduction in peripheral expression and an increase in neuronal tropism. Among the selected 5 AAV.FUS candidates, four transduced target brain sites more efficiently than AAV9 while also lowering transgene expression in the liver in the same mice. the top candidate, which is termed AAV.FUS.3 herein, demonstrated improved transduction in five different brain regions and an overall efficiency of targeting the brain, defined as the ratio of brain to liver (peripheral) transduction, improved 12.1-fold compared to AAV9. This improvement in tissue specificity is particularly important because peripheral transduction can lead to toxicity. For example, AAV-based gene therapy has been shown to induce liver toxicity in clinical trials.

The results suggest the need to investigate the mechanisms by which AAVs enter the brain after FUS-BBBO and what accounts for the differences in efficiency among serotypes. The prevailing understanding of FUS-BBBO mechanisms suggests that FUS loosens tight junctions in the vasculature, allowing molecules and nanoparticles such as AAVs to pass from the blood into the brain. Within this framework, reductions in peripheral uptake (leaving more AAV to circulate) and reduced binding to extracellular matrix could help certain serotypes enter through physically-generated openings and reach neurons more efficiently. Another possibility is that FUS-BBBO could cause molecular changes to the vascular endothelium, leading to a more complex interaction between viral vectors and their target. Understanding these factors would enable additional future engineering and optimization of FUS-BBBO-based gene delivery.

Overall, this study shows that the molecular engineering of AAV capsids can lead to improved ultrasound-mediated gene delivery to the brain. The screen yielded AAV.FUS.3, the first viral vector expressly engineered to work in conjunction with a specific physical delivery method.

### Materials and Methods

Animals

Animals. C57BL/6J and Syn-1-Cre mice were obtained from Jackson Lab. Animals were housed in a 12h light/dark cycle and were provided with water and food ad libitum. All experiments were conducted under a protocol approved by the Institutional Animal Care and Use Committee (IACUC) of the California Institute of Technology.

Focused Ultrasound Equipment and BBB Opening Procedures

FUS-BBBO. Adult male Syn1-Cre mice (at least 14 weeks old) were anesthetized with 2% isoflurane in air, the hair on their head removed with Nair depilation cream and then cannulated in the tail vein using a 30-gauge needle connected to PE10 tubing. The cannula was then flushed with 10 units (U)/ml of heparin in sterile saline (0.9% NaCl) and attached to the mouse tail using tissue glue (Gluture). Subsequently, the mice were placed in the custom-made plastic head mount and imaged in a 7T MRI (Bruker Biospec). A fast low-angle shot sequence (echo time TE=3.9 ms, repetition time TR=15 ms, flip angle 20°) was used to record the position of the ultrasound transducer in relation to the mouse brain. Subsequently, the mice were injected via tail vein with AAVs. Immediately after viral injection, the mice were also injected with 1.5×10⁶DEFINITY microbubbles (Lantheus) and 0.125 μmol of ProHance (Bracco Imaging) dissolved in sterile saline, per g of body weight. The dose of DEFINITY was identical as used in previous studies. The dose of ProHance was chosen based on the manufacturer's recommendations. Within 30 s, the mice were insonated using an eight-channel FUS system (Image Guided Therapy) driving an eight-element annular array transducer with a diameter of 25 mm and a natural focal point of 20 mm, coupled to the head via Aquasonic ultrasound gel. The gel was placed on the top and both sides of the animal's head to minimize reverberations from tissue/air interfaces. The focal distance was adjusted electronically. The ultrasound parameters used were 1.5 MHz, 1% duty cycle, and 1 Hz pulse repetition frequency for 120 pulses and were derived from a published protocol. The pressure was calibrated using a fiber optic hydrophone (Precision Acoustics), with 21 measurements and uncertainty of ±3.8% (SEM). The pressure for FUS-BBBO was chosen to maximize the safety of delivery and was chosen on the basis of previous studies and preliminary data in the laboratory. The ultrasound parameters were 1.5 MHz, 0.33 MPa pressure accounting for skull attenuation (18%), 1% duty cycle, and 1 Hz pulse repetition frequency for 120 pulses. For each FUS site, DEFINITY and Prohance were re-injected before the additional insonation. Each animal underwent four insonations located in one hemisphere, starting from the midbrain and going forward. The time between each insonation was approximately 3 minutes and included 120 s of insonation and 1 minute for readjustment of positioning on the stereotaxic frame. The center focus of beams was separated by 1.35-1.5 mm (depending on mouse weight 25-35 g) in the anterior/posterior direction.

Plasmids and DNA Library Generation

The plasmids used were either obtained from Addgene, Caltech's vector core, or modified from these plasmids. The AAV library genome used for selection (acceptor plasmid, rAAV9Rx/a-delta-CAP) was obtained from Caltech's vector core facility, as were other plasmids (REP2-CAP9Stop-DeltaX/A, pUC18). The Rep-Cap plasmid for packaging AAV.FUS candidates were modified from Addgene plasmid #103005 by introducing mutations selected from the screen. For testing the transduction a plasmid obtained from Addgene (pAAV-CaG-NLS-EGFP-#104061) and a plasmid modified in-house with exchanged EGFP for mCherry protein (pAAV-CaG-NLS-mCherry) was used.

Mutations were introduced into the acceptor plasmid using a PCR with degenerated primers (7MNN) with a sequence 5′-GTATTCCTTGGTTTTGAACCCAACCGGTCTGCGCCTGTGC NNTTGGGCACTCTGGTGGTTTGTG-3′ (SEQ ID NO: 21), targeted as a 7-aminoacid insertion between residues 587 and 588. The amplified insert was then introduced into the capsid plasmid through restriction cloning using XbaI and AgeI enzymes. DNA from the treated brain was recovered by PCR using two pairs of plasmids—the first step of amplification was done using 5′-CAGGTCTTCACGGACTCAGACTATCAG-3′ (SEQ ID NO: 17) and 5′-CAAGTAAAACCTCTACAAATGTGGTAAAATCG-3′ (SEQ ID NO: 18) primers which selected for the DNA that has been modified by Cre enzyme. The second stage, intended to amplify the DNA was performed using a pair of primers: 5′-ACTCATCGACCAATACTTGTACTATCTCTCTAGAAC-3′ (SEQ ID NO: 19) and 5′-GGAAGTATTCCTTGGTTTTGAACCCAA-3′ (SEQ ID NO: 20).

Virus Production and Purification

AAV library was purified as previously published. In short, the DNA carrying a genome containing capsid which has been modified by the 7-mer insertion (10 ng per 100 mm diameter dish) was transfected, the helper DNA containing REP protein (10 mg per 100 mm diameter dish, and 9.99 mg of empty pUC19 carrier plasmid), and an AdV helper plasmid (20 mg per 100 mm diameter dish) using PEI. Media was changed 16 h after transfection, and then collected 48 h post-transfection and stored in 4 C. 60h after the transfection, the cells were scraped into San digestion buffer (Tris pH 8.5 with 500 mM NaCl and 40 mM MgCl2 with Salt Active Nuclease). Virus in the media was precipitated using ⅕ volume of 5×PEG8000+NaCl (40% PEG-8000 and 2.5M NaCl), incubated on ice for 2 h, and spun at 3000×g for 30 min at 4 C. The media and cell-scraped stocks were then combined and precipitated using iodixanol gradient precipitation (virus appears on the 40-60% iodixanol interface), diluted into 15 ml PBS with 0.001% Pluronic-F68, and sterile-filtered through a 0.2-micron PES filter. Finally, the buffer was dialyzed using Amicon 100 KDa cut-off centrifuge filters at least 3 times to remove residual iodixanol, after which the virus was tittered using a standard qPCR protocol. AAV.FUS candidates were packaged and titered using a commercial service (Vigene biosciences) to ensure reproducibility for external investigators, as the titers can show variability between different labs. The AAV.FUS.3 and AAV9 were re-titered from another batch again in the lab, to make sure that the improvement of AAV.FUS over AAV9 is consistent between investigators.

In Vivo Selection and Gene Delivery

To enable in vivo selection of AAV.FUS the AAV library was delivered to one hemisphere through FUS-BBBO. Four sites corresponding to the striatum, dorsal hippocampus, ventral hippocampus, and midbrain were targeted using MRI guidance. 0.33 MPa pressure and other parameters were used as described in the Focused ultrasound equipment and BBB opening procedures section. The parameters used were identical during the in vivo selection and testing of the AAV.FUS candidates. The AAVs were delivered intravenously. For the first round of selection the dose delivered was 6.7E9 viral particles per gram of body weight. The library for the first round of evolution contained 1.3E9 sequences, yielding approximately 5 particles of each clone per gram of body weight. For the second round, where the library contained 2098 candidates, 1.3E9 viral particles per gram of body weight were delivered, yielding 6.2E5 viral particles for each clone per gram of body weight. After FUS-BBBO mice were returned to the home cages for 14 days, after which they were euthanized by CO² overdose.

Tissue Preparation for DNA Extraction

The brains of mice euthanized with CO² overdose were extracted, and the targeted hemisphere was separated from the control hemisphere with a clean blade. Each hemisphere was then frozen at −20 C prior to DNA extraction. The brains were then homogenized in Trizol using a BeadBug tissue homogenization device with dedicated pre-filled 2.0 ml tubes with beads (Zirconium coated, 1.5 mm, Benchmark Scientific, Sayreville, N.J.) for 1-3 minutes until tissue solution was homogenous. The DNA was then extracted with Trizol and amplified first with CRE-independent, and then CRE-dependent PCR as previously published.

Next Generation Sequencing Data Analysis

Next generation sequencing was performed using MiSeq (Illumina, San Diego, Calif.) using paired-end 75-base pair reads. The variable region of all detected capsid sequences was extracted from raw fastq files using the awk tool in Unix terminal. This process filtered out sequences not containing the constant 19 bp region flanking each side of the variable region. Sequences were then sorted, checked for length, and ordered from highest to lowest copy number in the sequencing experiment. During the first screen, the top 3000 were chosen. Among these 3000, any sequence that was only a point mutation away from a sequence and 30× less abundant was removed and assumed to be a potential sequencing readout error. This led to the final library of 2098 sequences, which were synthesized by Twist Biosciences (San Francisco, Calif.) for use in the second round of screening. This second AAV library also included a set of 2098 “codon-optimized” capsid variants that were encoded for the same protein as the original sequences but using a different DNA sequence chosen by the IDT codon optimization tool. To process the second batch of sequencing data, the copy numbers of the sequences in each experiment were first normalized to one to ensure comparability of different samples. Then, sequences were filtered out that were not contained within the input library. Finally, the normalized frequency of reads for each sequence, defined as the normalized copy number of each sequence averaged among original and codon-optimized variants for each capsid was evaluated. Top sequences for further analysis were selected to be the most abundant sequences that appeared at least 100× more frequently in the targeted brain hemisphere than the non-targeted hemisphere in all tested mice, and from these sequences, the top 5 were chosen as AAV.FUS candidates.

Histology, and Image Processing

After cardiac perfusion and extraction brains were post-fixed for 24 h in neutral buffered formalin (NBF). Brains were then sectioned at 50-micron on Compresstome VF-300 (Precisionary Instruments, Natick, Mass.). Sections were immunostained with an anti-NeuN Alexa Fluor 405-conjugated antibody (RBFOX3/NeuN Antibody by Novus Biological, Littleton, Colo., USA, stock number: NBP1-92693AF405). Sections were imaged on a Zeiss LSM-800 microscope using a 20× objective. Channels' laser intensities normalized to the brightness of mCherry and GFP proteins. Images were then randomized, anonymized, and exported as greyscale to ensure a lack of bias in color perception. The experimenter was blinded in terms of fluorophore color, tested AAV strain, or the mouse identification (H.L.). Three 100-micron sections of the brain were analyzed for each mouse, for each strain of the AAV including the section at the center of the FUS-target and the sections 500 and 1000 microns anterior to that section. The data was then independently validated by an experimenter blinded to the goals of the study (J.T). The inter-experimenter variability was 12.5% (1.9-fold (RL, primary scorer) vs 2.1-fold difference (JT, secondary scorer), n=15 randomly selected images, a total of 11,230 cells counted) and the difference between the scores was not statistically significant (p=0.071, two-tailed, paired t-test). To evaluate the BBB permeability of the AAV in the absence of FUS-BBBO (off-target transduction), a randomly chosen untargeted region at least 2 mm from the center of targeted region (4 times the distance of distance half-width half maximum of pressure, resulting in ^{˜}16-fold pressure reduction) was used within the same sections that were used to evaluate transduction efficiency at FUS focus.

Statistical Analysis

Two tailed t-test, without assuming equal variance, was used when comparing means of two data sets. For comparison of more than two data sets, one-way ANOVA was used, with a Tukey's HSD post-hoc test to determine significance of pairwise comparisons. When more than one variable was compared across multiple samples, two-way ANOVA was used, followed by Sidak's multiple comparisons test.

Sequences

Table 3 provides the amino acid sequences inserted into AAV9 capsid to obtain AAV.FUS.1-5 vectors. Table 4 provides amino acid sequences of AAV.FUS capsid proteins. Table 5 provides the DNA sequence of an ORF encoding the CAP protein of AAV.FUS.3 and the DNA sequences of the five AAV.FUS candidate insertions.

TABLE 3
7-mer Insertions

| Name | SEQ ID NO | Sequence |
|---|---|---|
| AAV.FUS.1 | 1 | AGNTSDR |
| AAV.FUS.2 | 2 | ATDAYNK |
| AAV.FUS.3 | 3 | WSEGGQP |
| AAV.FUS.4 | 4 | SVGSADP |
| AAV.FUS.5 | 5 | VRMEGEV |

TABLE 4
Amino Acid Sequences of AAV.FUS Capsid Proteins

| | SEQ ID | |
|---|---|---|
| Name | NO | Sequence |
| AAV.FUS. 1 | 6 | MAADGYLPDWLEDNLSEGIREWWALKPGAPQPKANQQHQDNARGL |
| Capsid | | VLPGYKYLGPGNGLDKGEPVNAADAAALEHDKAYDQQLKAGDNPY |
| | | LKYNHADAEFQERLKEDTSFGGNLGRAVFQAKKRLLEPLGLVEEAA |
| | | KTAPGKKRPVEQSPQEPDSSAGIGKSGAQPAKKRLNFGQTGDTESVP |
| | | DPQPIGEPPAAPSGVGSLTMASGGGAPVADNNEGADGVGSSSGNWH |
| | | CDSQWLGDRVITTSTRTWALPTYNNHLYKQISNSTSGGSSNDNAYFG |
| | | YSTPWGYFDFNRFHCHFSPRDWQRLINNNWGFRPKRLNFKLFNIQVK |
| | | EVTDNNGVKTIANNLTSTVQVFTDSDYQLPYVLGSAHEGCLPPFPAD |
| | | VFMIPQYGYLTLNDGSQAVGRSSFYCLEYFPSQMLRTGNNFQFSYEF |
| | | ENVPFHSSYAHSQSLDRLMNPLIDQYLYYLSRTINGSGQNQQTLKFSV |
| | | AGPSNMAVQGRNYIPGPSYRQQRVSTTVTQNNNSEFAWPGASSWAL |
| | | NGRNSLMNPGPAMASHKEGEDRFFPLSGSLIFGKQGTGRDNVDADK |
| | | VMITNEEEIKTTNPVATESYGQVATNHQSAQAGNTSDRAQAQTGWV |
| | | QNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKEN |
| | | SKRWNPEIQYTSNYYKSNNVEFAVNTEGVYSEPRPIGTRYLTRNL |
| AAV.FUS.2 | 7 | MAADGYLPDWLEDNLSEGIREWWALKPGAPQPKANQQHQDNARGL |
| Capsid | | VLPGYKYLGPGNGLDKGEPVNAADAAALEHDKAYDQQLKAGDNPY |
| | | LKYNHADAEFQERLKEDTSFGGNLGRAVFQAKKRLLEPLGLVEEAA |
| | | KTAPGKKRPVEQSPQEPDSSAGIGKSGAQPAKKRLNFGQTGDTESVP |
| | | DPQPIGEPPAAPSGVGSLTMASGGGAPVADNNEGADGVGSSSGNWH |
| | | CDSQWLGDRVITTSTRTWALPTYNNHLYKQISNSTSGGSSNDNAYFG |
| | | YSTPWGYFDFNRFHCHFSPRDWQRLINNNWGFRPKRLNFKLFNIQVK |
| | | EVTDNNGVKTIANNLTSTVQVFTDSDYQLPYVLGSAHEGCLPPFPAD |
| | | VFMIPQYGYLTLNDGSQAVGRSSFYCLEYFPSQMLRTGNNFQFSYEF |
| | | ENVPFHSSYAHSQSLDRLMNPLIDQYLYYLSRTINGSGQNQQTLKFSV |
| | | AGPSNMAVQGRNYIPGPSYRQQRVSTTVTQNNNSEFAWPGASSWAL |
| | | NGRNSLMNPGPAMASHKEGEDRFFPLSGSLIFGKQGTGRDNVDADK |
| | | VMITNEEEIKTTNPVATESYGQVATNHQSAQATDAYNKAQAQTGWV |
| | | QNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKEN |
| | | SKRWNPEIQYTSNYYKSNNVEFAVNTEGVYSEPRPIGTRYLTRNL |
| AAV.FUS.3 | 8 | MAADGYLPDWLEDNLSEGIREWWALKPGAPQPKANQQHQDNARGL |
| Capsid | | VLPGYKYLGPGNGLDKGEPVNAADAAALEHDKAYDQQLKAGDNPY |
| | | LKYNHADAEFQERLKEDTSFGGNLGRAVFQAKKRLLEPLGLVEEAA |
| | | KTAPGKKRPVEQSPQEPDSSAGIGKSGAQPAKKRLNFGQTGDTESVP |
| | | DPQPIGEPPAAPSGVGSLTMASGGGAPVADNNEGADGVGSSSGNWH |
| | | CDSQWLGDRVITTSTRTWALPTYNNHLYKQISNSTSGGSSNDNAYFG |
| | | YSTPWGYFDFNRFHCHFSPRDWQRLINNNWGFRPKRLNFKLFNIQVK |
| | | EVTDNNGVKTIANNLTSTVQVFTDSDYQLPYVLGSAHEGCLPPFPAD |
| | | VFMIPQYGYLTLNDGSQAVGRSSFYCLEYFPSQMLRTGNNFQFSYEF |
| | | ENVPFHSSYAHSQSLDRLMNPLIDQYLYYLSRTINGSGQNQQTLKFSV |
| | | AGPSNMAVQGRNYIPGPSYRQQRVSTTVTQNNNSEFAWPGASSWAL |
| | | NGRNSLMNPGPAMASHKEGEDRFFPLSGSLIFGKQGTGRDNVDADK |
| | | VMITNEEEIKTTNPVATESYGQVATNHQSAQWSEGGQPAQAQTGW |
| | | VQNQGILPGMVWQDRDVYLQGPIWAKIPHTDGNFHPSPLMGGFGMK |
| | | HPPPQILIKNTPVPADPPTAFNKDKLNSFITQYSTGQVSVEIEWELQKE |
| | | NSKRWNPEIQYTSNYYKSNNVEFAVNTEGVYSEPRPIGTRYLTRNL |
| AAV.FUS.4 | 9 | MAADGYLPDWLEDNLSEGIREWWALKPGAPQPKANQQHQDNARGL |
| Capsid | | VLPGYKYLGPGNGLDKGEPVNAADAAALEHDKAYDQQLKAGDNPY |
| | | LKYNHADAEFQERLKEDTSFGGNLGRAVFQAKKRLLEPLGLVEEAA |
| | | KTAPGKKRPVEQSPQEPDSSAGIGKSGAQPAKKRLNFGQTGDTESVP |
| | | DPQPIGEPPAAPSGVGSLTMASGGGAPVADNNEGADGVGSSSGNWH |
| | | CDSQWLGDRVITTSTRTWALPTYNNHLYKQISNSTSGGSSNDNAYFG |
| | | YSTPWGYFDFNRFHCHFSPRDWQRLINNNWGFRPKRLNFKLFNIQVK |
| | | EVTDNNGVKTIANNLTSTVQVFTDSDYQLPYVLGSAHEGCLPPFPAD |
| | | VFMIPQYGYLTLNDGSQAVGRSSFYCLEYFPSQMLRTGNNFQFSYEF |
| | | ENVPFHSSYAHSQSLDRLMNPLIDQYLYYLSRTINGSGQNQQTLKFSV |
| | | AGPSNMAVQGRNYIPGPSYRQQRVSTTVTQNNNSEFAWPGASSWAL |
| | | NGRNSLMNPGPAMASHKEGEDRFFPLSGSLIFGKQGTGRDNVDADK |
| | | VMITNEEEIKTTNPVATESYGQVATNHQSAQSVGSADPAQAQTGWV |
| | | QNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKEN |
| | | SKRWNPEIQYTSNYYKSNNVEFAVNTEGVYSEPRPIGTRYLTRNL |
| AAV.FUS.5 | 10 | MAADGYLPDWLEDNLSEGIREWWALKPGAPQPKANQQHQDNARGL |
| Capsid | | VLPGYKYLGPGNGLDKGEPVNAADAAALEHDKAYDQQLKAGDNPY |
| | | LKYNHADAEFQERLKEDTSFGGNLGRAVFQAKKRLLEPLGLVEEAA |
| | | KTAPGKKRPVEQSPQEPDSSAGIGKSGAQPAKKRLNFGQTGDTESVP |
| | | DPQPIGEPPAAPSGVGSLTMASGGGAPVADNNEGADGVGSSSGNWH |
| | | CDSQWLGDRVITTSTRTWALPTYNNHLYKQISNSTSGGSSNDNAYFG |
| | | YSTPWGYFDFNRFHCHFSPRDWQRLINNNWGFRPKRLNFKLFNIQVK |
| | | EVTDNNGVKTIANNLTSTVQVFTDSDYQLPYVLGSAHEGCLPPFPAD |
| | | VFMIPQYGYLTLNDGSQAVGRSSFYCLEYFPSQMLRTGNNFQFSYEF |
| | | ENVPFHSSYAHSQSLDRLMNPLIDQYLYYLSRTINGSGQNQQTLKFSV |
| | | AGPSNMAVQGRNYIPGPSYRQQRVSTTVTQNNNSEFAWPGASSWAL |
| | | NGRNSLMNPGPAMASHKEGEDRFFPLSGSLIFGKQGTGRDNVDADK |
| | | VMITNEEEIKTTNPVATESYGQVATNHQSAQVRMEGEVAQAQTGW |
| | | VQNQGILPGMVWQDRDVYLQGPIWAKIPHTDGNFHPSPLMGGFGMK |
| | | HPPPQILIKNTPVPADPPTAFNKDKLNSFITQYSTGQVSVEIEWELQKE |
| | | NSKRWNPEIQYTSNYYKSNNVEFAVNTEGVYSEPRPIGTRYLTRNL |

*Insertion of 7-mer indicated in bold

TABLE 5
DNA Sequence of ORF encoding the CAP protein of AAV.FUS.3 and DNA Sequences
of AAV.FUS Insertions

| | SEQ ID | |
|---|---|---|
| Name | NO | Sequence |
| AAV.FUS.3 | 11 | ATGGCTGCCGATGGTTATCTTCCAGATTGGCTCGAGGACAACCTTA |
| DNA | | GTGAAGGAATTCGCGAGTGGTGGGCTTTGAAACCTGGAGCCCCTC |
| | | AACCCAAGGCAAATCAACAACATCAAGACAACGCTCGAGGTCTTG |
| | | TGCTTCCGGGTTACAAATACCTTGGACCCGGCAACGGACTCGACA |
| | | AGGGGGAGCCGGTCAACGCAGCAGACGCGGCGGCCCTCGAGCAC |
| | | GACAAGGCCTACGACCAGCAGCTCAAGGCCGGAGACAACCCGTA |
| | | CCTCAAGTACAACCACGCCGACGCCGAGTTCCAGGAGCGGCTCAA |
| | | AGAAGATACGTCTTTTGGGGGCAACCTCGGGCGAGCAGTCTTCCA |
| | | GGCCAAAAAGAGGCTTCTTGAACCTCTTGGTCTGGTTGAGGAAGC |
| | | GGCTAAGACGGCTCCTGGAAAGAAGAGGCCTGTAGAGCAGTCTCC |
| | | TCAGGAACCGGACTCCTCCGCGGGTATTGGCAAATCGGGTGCACA |
| | | GCCCGCTAAAAAGAGACTCAATTTCGGTCAGACTGGCGACACAGA |
| | | GTCAGTCCCAGACCCTCAACCAATCGGAGAACCTCCCGCAGCCCC |
| | | CTCAGGTGTGGGATCTCTTACAATGGCTTCAGGTGGTGGCGCACC |
| | | AGTGGCAGACAATAACGAAGGTGCCGATGGAGTGGGTAGTTCCTC |
| | | GGGAAATTGGCATTGCGATTCCCAATGGCTGGGGGACAGAGTCAT |
| | | CACCACCAGCACCCGAACCTGGGCCCTGCCCACCTACAACAATCA |
| | | CCTCTACAAGCAAATCTCCAACAGCACATCTGGAGGATCTTCAAA |
| | | TGACAACGCCTACTTCGGCTACAGCACCCCCTGGGGGTATTTTGAC |
| | | TTCAACAGATTCCACTGCCACTTCTCACCACGTGACTGGCAGCGAC |
| | | TCATCAACAACAACTGGGGATTCCGGCCTAAGCGACTCAACTTCA |
| | | AGCTCTTCAACATTCAGGTCAAAGAGGTTACGGACAACAATGGAG |
| | | TCAAGACCATCGCCAATAACCTTACCAGCACGGTCCAGGTCTTCA |
| | | CGGACTCAGACTATCAGCTCCCGTACGTGCTCGGGTCGGCTCACG |
| | | AGGGCTGCCTCCCGCCGTTCCCAGCGGACGTTTTCATGATTCCTCA |
| | | GTACGGGTATCTGACGCTTAATGATGGAAGCCAGGCCGTGGGTCG |
| | | TTCGTCCTTTTACTGCCTGGAATATTTCCCGTCGCAAATGCTAAGA |
| | | ACGGGTAACAACTTCCAGTTCAGCTACGAGTTTGAGAACGTACCT |
| | | TTCCATAGCAGCTACGCTCACAGCCAAAGCCTGGACCGACTAATG |
| | | AATCCACTCATCGACCAATACTTGTACTATCTCTCTAGAACTATTA |
| | | ACGGTTCTGGACAGAATCAACAAACGCTAAAATTCAGTGTGGCCG |
| | | GACCCAGCAACATGGCTGTCCAGGGAAGAAACTACATACCTGGAC |
| | | CCAGCTACCGACAACAACGTGTCTCAACCACTGTGACTCAAAACA |
| | | ACAACAGCGAATTTGCTTGGCCTGGAGCTTCTTCTTGGGCTCTCAA |
| | | TGGACGTAATAGCTTGATGAATCCTGGACCTGCTATGGCCAGCCA |
| | | CAAAGAAGGAGAGGACCGTTTCTTTCCTTTGTCTGGATCTTTAATT |
| | | TTTGGCAAACAAGGAACTGGAAGAGACAACGTGGATGCGGACAA |
| | | AGTCATGATAACCAACGAAGAAGAAATTAAAACTACTAACCCGGT |
| | | AGCAACGGAGTCCTATGGACAAGTGGCCACAAACCACCAGAGTGC |
| | | CCAATGGAGCGAGGGCGGCCAGCCCGCACAGGCGCAGACCGGT |
| | | FGGGTTCAAAACCAAGGAATACTTCCGGGTATGGTTTGGCAGGAC |
| | | AGAGATGTGTACCTGCAAGGACCCATTTGGGCCAAAATTCCTCAC |
| | | ACGGACGGCAACTTTCACCCTTCTCCGCTGATGGGAGGGTTTGGA |
| | | ATGAAGCACCCGCCTCCTCAGATCCTCATCAAAAACACACCTGTA |
| | | CCTGCGGATCCTCCAACGGCCTTCAACAAGGACAAGCTGAACTCT |
| | | FTCATCACCCAGTATTCTACTGGCCAAGTCAGCGTGGAGATCGAGT |
| | | GGGAGCTGCAGAAGGAAAACAGCAAGCGCTGGAACCCGGAGATC |
| | | CAGTACACTTCCAACTATTACAAGTCTAATAATGTTGAATTTGCTG |
| | | FTAATACTGAAGGTGTATATAGTGAACCCCGCCCCATTGGCACCA |
| | | GATACCTGACTCGTAATCTGTAA |
| FUS.1 insert | 12 | GCGGGGAATACTAGTGATCGG |
| FUS.2 insert | 13 | GCCACCGACGCCTACAACAAG |
| FUS.3 insert | 14 | TGGAGCGAGGGCGGCCAGCCC |
| FUS.4 insert | 15 | AGCGTGGGCAGCGCCGACCCC |
| FUS.5 insert | 16 | GTGCGGATGGAGGGTGAGGTG |

*Bold region in SEQ ID NO: 11 indicates the site of insertion of FUS.3 insert
**SEQ ID NOS: 12-16 can be inserted after 1764th nucleotide, 588th residue of parental AAV capsid gene

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. An adeno-associated virus (AAV) acoustic targeting peptide comprising an amino acid sequence that comprises at least 4 contiguous amino acids from a sequence selected from the group consisting of AGNTSDR (SEQ ID NO: 1), ATDAYNK (SEQ ID NO: 2), WSEGGQP (SEQ ID NO: 3), SVGSADP (SEQ ID NO: 4), and VRMEGEV (SEQ ID NO: 5).

2. The AAV acoustic targeting peptide of claim 1, wherein the AAV acoustic targeting peptide comprises AGNTSDR (SEQ ID NO: 1).

3. The AAV acoustic targeting peptide of claim 1, wherein the AAV acoustic targeting peptide comprises ATDAYNK (SEQ ID NO: 2).

4. The AAV acoustic targeting peptide of claim 1, wherein the AAV acoustic targeting peptide comprises WSEGGQP (SEQ ID NO: 3).

5. The AAV acoustic targeting peptide of claim 1, wherein the AAV acoustic targeting peptide comprises SVGSADP (SEQ ID NO: 4).

6. The AAV acoustic targeting peptide of claim 1, wherein the AAV acoustic targeting peptide comprises VRMEGEV (SEQ ID NO: 5).

7. The AAV acoustic targeting peptide of claim 1, wherein the AAV acoustic targeting peptide is part of a capsid protein of an AAV.

8. The AAV acoustic targeting peptide of claim 1, wherein the AAV acoustic targeting peptide is conjugated to a nanoparticle, a second molecule, a viral capsid protein, or a combination thereof.

9. The AAV acoustic targeting peptide of claim 1, wherein the AAV acoustic targeting peptide is configured enhance the focused ultrasound (FUS)-target specificity of FUS blood-brain-barrier opening (FUS-BBBO)-mediated transduction.

10. The AAV acoustic targeting peptide of claim 1, wherein an AAV comprising the AAV acoustic targeting peptide demonstrates
an at least about 1.1-fold increase in transduction at site(s) of FUS-BBBO;
an at least about 1.1-fold increase in neuronal tropism; and/or
an at least about 1.1-fold decrease in transduction in peripheral organs
as compared to the corresponding parental AAV that does not comprise the acoustic targeting peptide.

11. An adeno-associated virus (AAV) capsid protein comprising the AAV acoustic targeting peptide of claim 1.

12. The AAV capsid protein of claim 11, wherein the AAV capsid is derived from AAV9, or a variant thereof.

13. A nucleic acid, comprising a sequence encoding the AAV acoustic targeting peptide of claim 1.

14. A recombinant adeno-associated virus (rAAV), comprising the AAV acoustic targeting peptide of claim 1.

15. A recombinant adeno-associated virus (rAAV), comprising an AAV capsid protein which comprises the AAV acoustic targeting peptide of claim 1, wherein the amino acid sequence is inserted between two adjacent amino acids in AA586-592, or functional equivalents thereof, of the AAV capsid protein.

16. The rAAV of claim 15, wherein the AAV capsid protein comprises, or consists thereof, SEQ ID NO: 6, SEQ ID NO: 7, SEQ ID NO: 8, SEQ ID NO: 9, and/or SEQ ID NO: 10.

17. A composition for use in the delivery of an agent to a target environment of a subject, comprising an AAV comprising (1) an AAV capsid protein comprising an AAV acoustic targeting peptide of claim 1 and (2) an agent to be delivered to the target environment of the subject, wherein the target environment comprises one or more target brain region(s), and wherein the agent to be delivered comprises a nucleic acid, a peptide, a small molecule, an aptamer, or a combination thereof.

18. A method of delivering an agent to one or more target brain region(s) of a subject, the method comprising:
providing an AAV vector comprising an AAV capsid protein comprising an AAV acoustic targeting peptide, wherein the AAV acoustic targeting peptide comprises an amino acid sequence that comprises at least 4 contiguous amino acids from a sequence selected from the group consisting of AGNTSDR (SEQ ID NO: 1), ATDAYNK (SEQ ID NO: 2), WSEGGQP (SEQ ID NO: 3), SVGSADP (SEQ ID NO: 4), and VRMEGEV (SEQ ID NO: 5), and wherein the AAV vector further comprises an agent to be delivered to the one or more target brain region(s); and
administering the AAV vector to the subject.

19. The method of claim 18, wherein the method further comprises:
administering to the subject an effective amount of a microbubble contrast agent; and
applying focused ultrasound (FUS) to the one or more target brain region(s) of the subject.

20. A method of treating or preventing a disease or disorder in a subject in need thereof, comprising:
administering to the subject an effective amount of an AAV vector comprising an AAV capsid protein comprising an AAV acoustic targeting peptide, wherein the AAV acoustic targeting peptide comprises an amino acid sequence that comprises at least 4 contiguous amino acids from a sequence selected from the group consisting of AGNTSDR (SEQ ID NO: 1), ATDAYNK (SEQ ID NO: 2), WSEGGQP (SEQ ID NO: 3), SVGSADP (SEQ ID NO: 4), and VRMEGEV (SEQ ID NO: 5), and wherein the AAV vector further comprises an agent to be delivered to the one or more target brain region(s);
administering to the subject an effective amount of a microbubble contrast agent; and
applying focused ultrasound (FUS) to one or more target brain region(s) of the subject,
thereby treating or preventing the disease or disorder in the subject.


SEQUENCE LISTING (21 sequences)

SEQ ID NO: 1 - protein, 7 aa, synthetic construct
  source 1..7: note = AAV.FUS.1
AGNTSDR

SEQ ID NO: 2 - protein, 7 aa, synthetic construct
  source 1..7: note = AAV.FUS.2
ATDAYNK

SEQ ID NO: 3 - protein, 7 aa, synthetic construct
  source 1..7: note = AAV.FUS.3
WSEGGQP

SEQ ID NO: 4 - protein, 7 aa, synthetic construct
  source 1..7: note = AAV.FUS.4
SVGSADP

SEQ ID NO: 5 - protein, 7 aa, synthetic construct
  source 1..7: note = AAV.FUS.5
VRMEGEV

SEQ ID NO: 6 - protein, 743 aa, synthetic construct
  source 1..743: note = AAV.FUS.1 Capsid
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAGNTSDRAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 7 - protein, 743 aa, synthetic construct
  source 1..743: note = AAV.FUS.2 Capsid
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQATDAYNKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 8 - protein, 743 aa, synthetic construct
  source 1..743: note = AAV.FUS.3 Capsid
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQWSEGGQPAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 9 - protein, 743 aa, synthetic construct
  source 1..743: note = AAV.FUS.4 Capsid
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQSVGSADPAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 743 aa, synthetic construct
  source 1..743: note = AAV.FUS.5 Capsid
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQVRMEGEVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - DNA, 2232 bp, synthetic construct
  source 1..2232: mol_type = other DNA; note = AAV.FUS.3 Capsid DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaatggagcgagggcggccagcccgcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 12 - DNA, 21 bp, synthetic construct
  source 1..21: mol_type = other DNA; note = FUS.1 insert
gcggggaatactagtgatcgg

SEQ ID NO: 13 - DNA, 21 bp, synthetic construct
  source 1..21: mol_type = other DNA; note = FUS.2 insert
gccaccgacgcctacaacaag

SEQ ID NO: 14 - DNA, 21 bp, synthetic construct
  source 1..21: mol_type = other DNA; note = FUS.3 insert
tggagcgagggcggccagccc

SEQ ID NO: 15 - DNA, 21 bp, synthetic construct
  source 1..21: mol_type = other DNA; note = FUS.4 insert
agcgtgggcagcgccgacccc

SEQ ID NO: 16 - DNA, 21 bp, synthetic construct
  source 1..21: mol_type = other DNA; note = FUS.5 insert
gtgcggatggagggtgaggtg

SEQ ID NO: 17 - DNA, 27 bp, synthetic construct
  source 1..27: mol_type = other DNA
caggtcttcacggactcagactatcag

SEQ ID NO: 18 - DNA, 32 bp, synthetic construct
  source 1..32: mol_type = other DNA
caagtaaaacctctacaaatgtggtaaaatcg

SEQ ID NO: 19 - DNA, 36 bp, synthetic construct
  source 1..36: mol_type = other DNA
actcatcgaccaatacttgtactatctctctagaac

SEQ ID NO: 20 - DNA, 27 bp, synthetic construct
  source 1..27: mol_type = other DNA
ggaagtattccttggttttgaacccaa

SEQ ID NO: 21 - DNA, 81 bp, synthetic construct
  source 1..81: mol_type = other DNA
gtattccttggttttgaacccaaccggtctgcgcctgtgcnmnnmnnmnnmnnmnnmnnttgggcactctggtggtttgtg